(19) United States
(12) Patent Application Publication
(10) Pub. No.: US 2023/0192722 A1
(43) Pub. Date: Jun. 22, 2023

(54) SALTS AND SOLID FORMS OF AN FGFR INHIBITOR AND PROCESSES OF PREPARING THEREOF

(71) Applicant: Incyte Corporation, Wilmington, DE (US)

(72) Inventors: Pingli Liu, Wilmington, DE (US); Zhongjiang Jia, Kennett Square, PA (US); Qiyan Lin, Newark, DE (US); Naijing Su, Hockessin, DE (US); Timothy Martin, Hockessin, DE (US); Michael Xia, Wilmington, DE (US); Jiacheng Zhou, Newark, DE (US); Baoyu Mi, Wilmington, DE (US)

(21) Appl. No.: 18/085,814

(22) Filed: Dec. 21, 2022

Related U.S. Application Data
(60) Provisional application No. 63/292,628, filed on Dec. 22, 2021.

(51) Int. Cl. C07D 519/00; C07D 471/04

(52) U.S. Cl. CPC C07D 519/00; C07D 471/04; C07B 2200/13

(57) ABSTRACT

The present invention relates to salts and solid forms of the FGFR inhibitor (7R,8aS)-2-(5-(5-(2,3-Dimethylphenyl)-6-methoxy-1H-pyrazolo[4,3-b]pyridin-3-yl)pyridin-2-yl)octahydropyrrolo[1,2-a]pyrazin-7-ol, including methods of preparation thereof, wherein the compounds, salts, and solid forms are useful in the treatment of FGFR-mediated diseases such as cancer.

### FIELD OF THE INVENTION

The present invention relates to salts and solid forms of the FGFR inhibitor (7R,8aS)-2-(5-(5-(2,3-Dimethylphenyl)-6-methoxy-1H-pyrazolo[4,3-b]pyridin-3-yl)pyridin-2-yl)octahydropyrrolo[1,2-a]pyrazin-7-ol, including methods of preparation thereof, wherein the compounds, salts, and solid forms are useful in the treatment of FGFR-mediated diseases such as cancer.

### BACKGROUND OF THE INVENTION

The Fibroblast Growth Factor Receptors (FGFR) are receptor tyrosine kinases that bind to fibroblast growth factor (FGF) ligands. There are four FGFR proteins (FGFR1-4) that are capable of binding ligands and are involved in the regulation of many physiological processes including tissue development, angiogenesis, wound healing, and metabolic regulation. Upon ligand binding, the receptors undergo dimerization and phosphorylation leading to stimulation of the protein kinase activity and recruitment of many intracellular docking proteins. These interactions facilitate the activation of an array of intracellular signaling pathways including Ras-MAPK, AKT-PI3K, and phospholipase C that are important for cellular growth, proliferation and survival (Reviewed in Eswarakumar et al. Cytokine & Growth Factor Reviews, 2005, 16, 139-149). Aberrant activation of this pathway either through overexpression of FGF ligands or FGFR or activating mutations in the FGFRs can lead to tumor development, progression, and resistance to conventional cancer therapies. In human cancer, genetic alterations including gene amplification, chromosomal translocations and somatic mutations that lead to ligand-independent receptor activation have been described (Reviewed in Knights and Cook, Pharmacology & Therapeutics, 2010, 125, 105-117; Turner and Grose, Nature Reviews Cancer, 2010, 10, 116-129). Large scale DNA sequencing of thousands of tumor samples has revealed that FGFR genes are altered in many cancers (Helsten et al. Clin Cancer Res. 2016, 22, 259-267). Some of these activating mutations are identical to germline mutations that lead to skeletal dysplasia syndromes (Gallo et al. Cytokine & Growth Factor Reviews 2015, 26, 425-449). Mechanisms that lead to aberrant ligand-dependent signaling in human disease include overexpression of FGFs and changes in FGFR splicing that lead to receptors with more promiscuous ligand binding abilities. Therefore, development of inhibitors targeting FGFR may be useful in the clinical treatment of diseases that have elevated FGF or FGFR activity.

The cancer types in which FGF/FGFRs are implicated include, but are not limited to: carcinomas (e.g., bladder, breast, colorectal, endometrial, gastric, head and neck, kidney, lung, ovarian, prostate); hematopoietic malignancies (e.g., multiple myeloma, acute myelogenous leukemia, and myeloproliferative neoplasms); and other neoplasms (e.g., glioblastoma and sarcomas). In addition to a role in oncogenic neoplasms, FGFR activation has also been implicated in skeletal and chondrocyte disorders including, but not limited to, achrondroplasia and craniosynostosis syndromes.

Inhibitors of FGFR are currently being developed for the treatment of cancer. For example, the molecule (7R,8aS)-2-(5-(5-(2,3-Dimethylphenyl)-6-methoxy-1H-pyrazolo[4,3-b]pyridin-3-yl)pyridin-2-yl)octahydropyrrolo[1,2-a]pyrazin-7-ol, and other small molecule inhibitors of FGFR are reported in e.g., US Patent Publication No. 2021/0106588. Accordingly, there is a need for new solid forms and salts of FGFR-inhibiting molecules for preparing pharmaceutically useful formulations and dosage forms with suitable properties related to, for example, facilitating the manufacture of safe, effective, and high quality drug products.

### SUMMARY OF THE INVENTION

The present invention is directed to solid forms of (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol (Compound 1) and salts thereof.

The present invention is further directed to salts of Compound 1. The present invention is further directed to the phosphoric acid salt of Compound 1, the hydrochloric acid salt of Compound 1, the L-(+)-tartaric acid salt of Compound 1, the malonic acid salt of Compound 1, the methanesulfonic acid salt of Compound 1, the adipic acid salt of Compound 1, the fumaric acid salt of Compound 1, the maleic acid salt of Compound 1, the malic acid salt of Compound 1, and the succinic acid salt of Compound 1.

The present invention is further directed to crystalline forms of the salts described herein.

The present invention is further directed to processes for preparing Compound 1 and its salts.

The present invention is further directed to pharmaceutical compositions comprising a salt or crystalline form described herein, and at least one pharmaceutically acceptable carrier.

The present invention is further directed to therapeutic methods of using the salts and solid forms described herein. The present disclosure also provides uses of the salts and solid forms described herein in the manufacture of a medicament for use in therapy. The present disclosure also provides the salts and solid forms described herein for use in therapy.

The present invention is further directed to processes for preparing the salts and solid forms described herein.

### BRIEF DESCRIPTION OF THE DRAWINGS

FIG. 1 shows an X-Ray Powder Diffraction (XRPD) pattern of Compound 1 Form I.

FIG. 2 shows a Differential Scanning calorimetry (DSC) thermogram of Compound 1 Form I.

FIG. 3 shows a Thermogravimetric Analysis (TGA) thermogram of Compound 1 Form I.

FIG. 4 shows an XRPD pattern of Compound 1 Form II.

FIG. 5 shows a DSC thermogram of Compound 1 Form II.

FIG. 6 shows a TGA thermogram of Compound 1 Form II.

FIG. 7 shows an atomic displacement ellipsoid diagram of Compound 1 Form II.

FIG. 8 shows a calculated XRPD pattern of Compound 1 Form II.

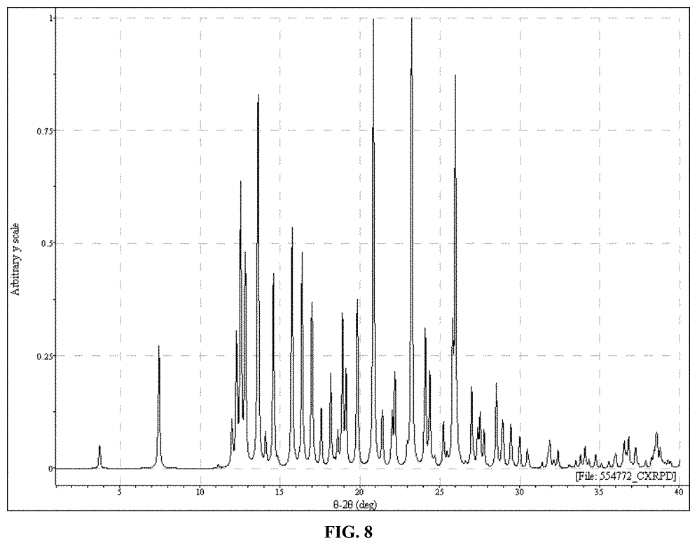

FIG. 9 shows an XRPD pattern of Compound 1 phosphate Form I.

FIG. 10 shows a DSC thermogram of Compound 1 phosphate Form I.

FIG. 11 shows a TGA thermogram of Compound 1 phosphate Form I.

FIG. 12 shows an XRPD pattern of Compound 1 phosphate Form II.

FIG. 13 shows a DSC thermogram of Compound 1 phosphate Form II.

FIG. 14 shows a TGA thermogram of Compound 1 phosphate Form II.

FIG. 15 shows an XRPD pattern of Compound 1 phosphate Form III.

FIG. 16 shows a DSC thermogram of Compound 1 phosphate Form III.

FIG. 17 shows a TGA thermogram of Compound 1 phosphate Form III.

FIG. 18 shows an XRPD pattern of Compound 1 hydrochloride Form I.

FIG. 19 shows a DSC thermogram of Compound 1 hydrochloride Form I.

FIG. 20 shows a TGA thermogram of Compound 1 hydrochloride Form I.

FIG. 21 shows an XRPD pattern of Compound 1 hydrochloride Form II.

FIG. 22 shows a DSC thermogram of Compound 1 hydrochloride Form II.

FIG. 23 shows a TGA thermogram of Compound 1 hydrochloride Form II.

FIG. 24 shows an XRPD pattern of Compound 1 L-tartrate.

FIG. 25 shows a DSC thermogram of Compound 1 L-tartrate.

FIG. 26 shows a TGA thermogram of Compound 1 L-tartrate.

FIG. 27 shows an XRPD pattern of Compound 1 malonate.

FIG. 28 shows a DSC thermogram of Compound 1 malonate.

FIG. 29 shows a TGA thermogram of Compound 1 malonate.

FIG. 30 shows an XRPD pattern of Compound 1 mesylate.

FIG. 31 shows a DSC thermogram of Compound 1 mesylate.

FIG. 32 shows a TGA thermogram of Compound 1 mesylate.

### DETAILED DESCRIPTION

The present invention is directed to, inter alia, salts and solid forms of (7R,8aS)-2-(5-(5-(2,3-dimethylphenyl)-6-methoxy-1H-pyrazolo[4,3-b]pyridin-3-yl)pyridin-2-yl)octahydropyrrolo[1,2-c]pyrazin-7-ol (Compound 1), the structure of which is shown below:

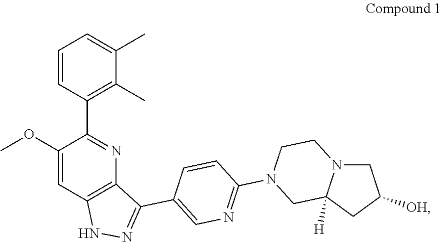 [SMILES: COc1cc2[nH]nc(-c3ccc(N4CCN5C[C@H](O)C[C@H]5C4)nc3)c2nc1-c1cccc(C)c1C]

Compound 1 is described in US Patent Publication No. 2021/0106588, the entirety of which is incorporated herein by reference.

Compound 1 and its salts can be isolated as one or more solid forms. The solid forms (e.g., crystalline forms) described herein have many advantages, for example they have desirable properties, such as ease of handling, ease of processing, storage stability, and ease of purification. Moreover, the crystalline forms can be useful for improving the performance characteristics of a pharmaceutical product such as dissolution profile, shelf-life and bioavailability.

In some embodiments, the salt of Compound 1 is an acid salt of Compound 1. In some embodiments, the acid is selected from phosphoric acid, hydrochloric acid, L-(+)-tartaric acid, malonic acid, methanesulfonic acid, adipic acid, fumaric acid, maleic acid, malic acid, and succinic acid.

In some embodiments, the salt of the invention is a phosphoric acid salt of Compound 1. The phosphoric acid salt of Compound 1 is referred to herein as “Compound 1 phosphate salt,” “Compound 1 phosphoric acid salt form,” “Compound 1 phosphoric acid,” or “Compound 1 phosphate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol phosphate.

In some embodiments, the salt of the invention is a hydrochloric acid salt of Compound 1. The hydrochloric acid salt form of Compound 1 is referred to herein as “Compound 1 hydrochloride salt,” “Compound 1 hydrochloric acid salt form,” “Compound 1 hydrochloric acid,” or “Compound 1 hydrochloride.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol hydrochloride.

In some embodiments, the salt of the invention is a L-(+)-tartaric acid salt of Compound 1. The L-(+)-tartaric acid salt form of Compound 1 is referred to herein as “Compound 1 L-tartrate salt,” “Compound 1 L-(+)-tartaric acid salt form,” “Compound 1 L-(+)-tartaric acid,” or “Compound 1 L-tartrate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol L-tartrate.

In some embodiments, the salt of the invention is a malonic acid salt (e.g., malonic acid salt) of Compound 1. The malonic acid salt form of Compound 1 is referred to herein as “Compound 1 malonate salt,” “Compound 1 malonic acid salt form,” “Compound 1 malonic acid,” or “Compound 1 malonate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol malonate.

In some embodiments, the salt of the invention is a methanesulfonic acid salt of Compound 1. The methanesulfonic acid salt form of Compound 1 is referred to herein as “Compound 1 mesylate salt,” “Compound 1 methanesulfonic acid salt form,” “Compound 1 methanesulfonic acid,” or “Compound 1 mesylate.” An alternative name for the salt is (7R,8aS)-2-(5-(5-(2,3-dimethylphenyl)-6-methoxy-1H-pyrazolo[4,3-b]pyridin-3-yl)pyridin yl)octahydropyrrolo[1,2-a]pyrazin-7-ol mesylate.

In some embodiments, the salt of the invention is an adipic acid salt of Compound 1. The adipic acid salt form of Compound 1 is referred to herein as “Compound 1 adipate salt,” “Compound 1 adipic acid salt form,” “Compound 1 adipic acid,” or “Compound 1 adipate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol adipate.

In some embodiments, the salt of the invention is a fumaric acid salt of Compound 1. The fumaric acid salt form of Compound 1 is referred to herein as “Compound 1 fumarate salt,” “Compound 1 fumaric acid salt form,” “Compound 1 fumaric acid,” or “Compound 1 fumarate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol fumarate.

In some embodiments, the salt of the invention is a maleic acid salt of Compound 1. The maleic acid salt form of Compound 1 is referred to herein as “Compound 1 maleate salt,” “Compound 1 maleic acid salt form,” “Compound 1 maleic acid,” or “Compound 1 maleate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol maleate.

In some embodiments, the salt of the invention is a malic acid salt (e.g., L-(−)-malic acid salt) of Compound 1. The malic acid salt form of Compound 1 is referred to herein as “Compound 1 malate salt,” “Compound 1 L-malate salt,” “Compound 1 malic acid salt form,” “Compound 1 malic acid,” “Compound 1 malate,” or “Compound 1 L-malate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol L-malate.

In some embodiments, the salt of the invention is a succinic acid salt of Compound 1. The succinic acid salt form of Compound 1 is referred to herein as “Compound 1 succinate salt,” “Compound 1 succinic acid salt form,” “Compound 1 succinic acid,” or “Compound 1 succinate.” An alternative name for the salt is (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol succinate.

The salts of the invention can be isolated as one or more solid forms. As used herein, the phrase “solid form” refers to a salt of the invention in either an amorphous state or a crystalline state (“crystalline form” or “crystalline solid”), whereby a salt of the invention in a crystalline state may optionally include solvent or water within the crystalline lattice, for example, to form a solvated or hydrated crystalline form. In some embodiments, the salt of the present invention is in a crystalline state as described herein. The term “hydrated,” as used herein, is meant to refer to a crystalline form that includes one or more water molecules in the crystalline lattice. Example “hydrated” crystalline forms include hemihydrates, monohydrates, dihydrates, and the like. Other hydrated forms such as channel hydrates and the like are also included within the meaning of the term.

In some embodiments, salts of the invention can be prepared by any suitable method for the preparation of acid addition salts. For example, the free base of a compound (e.g., Compound 1) can be combined with the desired acid in a solvent or in a melt. Alternatively, an acid addition salt of a compound can be converted to a different acid addition salt by anion exchange. Salts of the invention which are prepared in a solvent system can be isolated by precipitation from the solvent. Precipitation and/or crystallization can be induced, for example, by evaporation, reduction of temperature, addition of anti-solvent, or combinations thereof.

In some embodiments, the salts of the invention are crystalline, including crystalline forms which are anhydrous, hydrated, non-solvated, or solvated. Example hydrates include hemihydrates, monohydrates, dihydrates, and the like. In some embodiments, the crystalline salts are anhydrous and non-solvated. By “anhydrous” is meant that the crystalline salt contains no bound water in the crystal lattice structure, i.e., the compound does not form a crystalline hydrate.

In some embodiments, the salts of the invention are substantially isolated. By “substantially isolated” is meant that the salt is at least partially or substantially separated from the environment in which it was formed or detected. Partial separation can include, for example, a composition enriched in the salt of the invention. Substantial separation can include compositions containing at least about 50%, at least about 60%, at least about 70%, at least about 80%, at least about 90%, at least about 95%, at least about 97%, or at least about 99% by weight of the salt.

The salt forms of the invention were found to be highly crystalline, a desirable property which can facilitate, for example, purification of the drug such as by crystallization and recrystallization as necessary. Further, a crystalline form tends to be more stable and can be easier to mill or micronize when formulating a drug. Crystalline salts also tend have excellent properties with respect to solubility and can be more suitable to be manufactured reproducibly in a clear acid/base ratio, facilitating the preparation of liquid formulations for oral as well as for intravenous applications.

As used herein, the term “crystalline” or “crystalline form” refers to a crystalline solid form of a chemical compound, including, but not limited to, a single-component or multiple-component crystal form, e.g., including solvates, hydrates, clathrates, and a co-crystals. As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

Crystalline forms are most commonly characterized by XRPD. An XRPD pattern of reflections (peaks) is typically considered a fingerprint of a particular crystalline form. It is well known that the relative intensities of the XRPD peaks can widely vary depending on, inter alia, the sample preparation technique, crystal size distribution, filters, the sample mounting procedure, and the particular instrument employed. In some instances, new peaks may be observed or existing peaks may disappear, depending on the type of instrument or the settings (for example, whether a Ni filter is used or not). As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” as used in the context of XRPD herein is meant to encompass the above-mentioned variations.

In the same way, temperature readings in connection with DSC, TGA, or other thermal experiments can vary about ±3° C. depending on the instrument, particular settings, sample preparation, etc. For example, with DSC it is known that the temperatures observed will depend on the rate of the temperature change as well as the sample preparation technique and the particular instrument employed. Thus, the values reported herein related to DSC thermograms can vary, as indicated above, by ±3° C. Accordingly, a crystalline form reported herein having a DSC thermogram “substantially” as shown in any of the Figures is understood to accommodate such variation.

The salts and compounds disclosed herein can include all isotopes of atoms occurring within them. Isotopes include those atoms having the same atomic number but different mass numbers. For example, isotopes of hydrogen include tritium and deuterium. Salts and compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7 or 8 deuterium atoms. Synthetic methods for including isotopes into organic compounds are known in the art.

As used herein, and unless otherwise specified, the term “about”, when used in connection with a numeric value or range of values which is provided to describe a particular solid form (e.g., a specific temperature or temperature range, such as describing a melting, dehydration, or glass transition; a mass change, such as a mass change as a function of temperature or humidity; a solvent or water content, in terms of, for example, mass or a percentage; or a peak position, such as in analysis by, for example, ¹³C NMR, DSC, TGA and XRPD), indicate that the value or range of values may deviate to an extent deemed reasonable to one of ordinary skill in the art while still describing the particular solid form. Specifically, the term “about”, when used in this context, indicates that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values while still describing the particular solid form. The term “about”, when used in reference to a degree 2-theta value refers to +/−0.2 degrees 2-theta.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “melting point” refers to an endothermic event or endothermal event observed in e.g., a DSC experiment. An endothermic event is a process or reaction in which a sample absorbs energy from its surrounding in the form of e.g., heat as in a DSC experiment. An exothermic event is a process or reaction in which a sample releases energy. The process of heat absorption and release can be detected by DSC. In some embodiments, the term “melting point” is used to describe the major endothermic event revealed on a particular DSC thermogram.

The term “room temperature” as used herein, is understood in the art, and refers generally to a temperature, e.g., a reaction temperature, that is about the temperature of the room in which the reaction is carried out, for example, a temperature from about 20° C. to about 30° C. The term “elevated temperature” as used herein, is understood in the art, and refer generally to a temperature, e.g., a reaction temperature, that is above room temperature, e.g., above 30° C.

### Solid Forms of Compound 1

### Compound 1 Form I

Provided herein is a solid form of Compound 1 which is crystalline, referred to as Form I, or Compound 1 Form I, which is described below in the Examples.

Provided herein are also processes for preparing Form I comprising recrystallizing Compound 1 in a recrystallizing solvent. In some embodiments, the recrystallizing comprises precipitating Compound 1 Form I from a mixture of Compound 1 and the recrystallizing solvent. In some embodiments, the recrystallizing solvent is a polar solvent. In some embodiments, the recrystallizing solvent is a protic solvent. In some embodiments, the recrystallizing solvent is a polar aprotic solvent. In some embodiments, the recrystallizing solvent is water. In some embodiments, the recrystallizing solvent is tetrahydrofuran. In some embodiments, the recrystallizing solvent is a mixture of a protic solvent and a polar aprotic solvent. In some embodiments, the recrystallizing solvent is a mixture of water and tetrahydrofuran. In some embodiments, the precipitating is performed at a reduced temperature. In some embodiments, the reduced temperature is from about 10° C. to about 30° C. In some embodiments, the reduced temperature is room temperature.

In some embodiments, Form I has at least one characteristic XRPD peak selected from about 4.9, about 9.3, about 12.3, about 14.7, and about 16.3 degrees 2-theta. In some embodiments, Form I has at least two characteristic XRPD peaks selected from about 4.9, about 9.3, about 12.3, about 14.7, and about 16.3 degrees 2-theta. In some embodiments, Form I has at least three characteristic XRPD peaks selected from about 4.9, about 9.3, about 12.3, about 14.7, and about 16.3 degrees 2-theta. In some embodiments, Form I has at least four characteristic XRPD peaks selected from about 4.9, about 9.3, about 12.3, about 14.7, and about 16.3 degrees 2-theta. In some embodiments, Form I has at least five characteristic XRPD peaks selected from about 4.9, about 9.3, about 12.3, about 14.7, and about 16.3 degrees 2-theta. In some embodiments, Form I has characteristic XRPD peaks at about 4.9, about 9.3, about 12.3, about 14.7, and about 16.3 degrees 2-theta. In some embodiments, Form I has a characteristic XRPD peak at about 4.9 degrees 2-theta. In some embodiments, Form I has a characteristic XRPD peak at about 9.3 degrees 2-theta. In some embodiments, Form I has a characteristic XRPD peak at about 12.3 degrees 2-theta. In some embodiments, Form I has a characteristic XRPD peak at about 14.7 degrees 2-theta. In some embodiments, Form I has a characteristic XRPD peak at about 16.3 degrees 2-theta.

In some embodiments, Form I has at least one characteristic XRPD peak selected from about 4.9, about 9.3, about 12.3, about 14.7, about 16.3, about 17.8, about 19.4, about 20.5, about 21.9, about 24.4, and about 25.1 degrees 2-theta. In some embodiments, Form I has at least two characteristic XRPD peaks selected from about 4.9, about 9.3, about 12.3, about 14.7, about 16.3, about 17.8, about 19.4, about 20.5, about 21.9, about 24.4, and about 25.1 degrees 2-theta. In some embodiments, Form I has at least three characteristic XRPD peaks selected from about 4.9, about 9.3, about 12.3, about 14.7, about 16.3, about 17.8, about 19.4, about 20.5, about 21.9, about 24.4, and about 25.1 degrees 2-theta. In some embodiments, Form I has at least four characteristic XRPD peaks selected from about 4.9, about 9.3, about 12.3, about 14.7, about 16.3, about 17.8, about 19.4, about 20.5, about 21.9, about 24.4, and about 25.1 degrees 2-theta. In some embodiments, Form I has at least five characteristic XRPD peaks selected from about 4.9, about 9.3, about 12.3, about 14.7, about 16.3, about 17.8, about 19.4, about 20.5, about 21.9, about 24.4, and about 25.1 degrees 2-theta. In some embodiments, Form I has characteristic XRPD peaks at about 4.9, about 9.3, about 12.3, about 14.7, about 16.3, about 17.8, about 19.4, about 20.5, about 21.9, about 24.4, and about 25.1 degrees 2-theta.

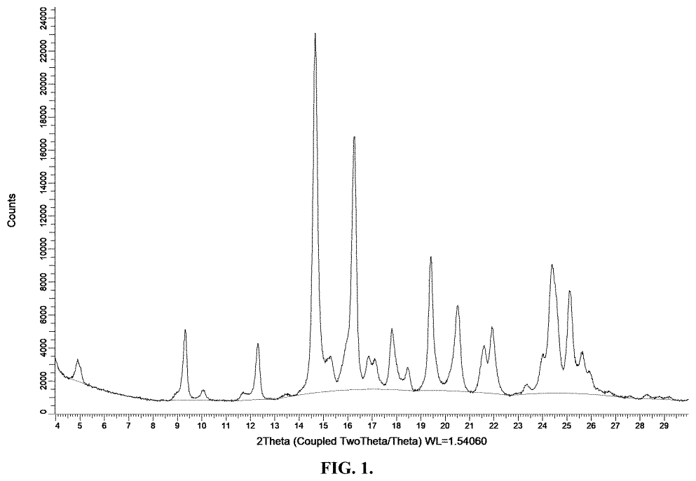

In some embodiments, Form I has an XRPD pattern with characteristic peaks as substantially shown in FIG. 1.

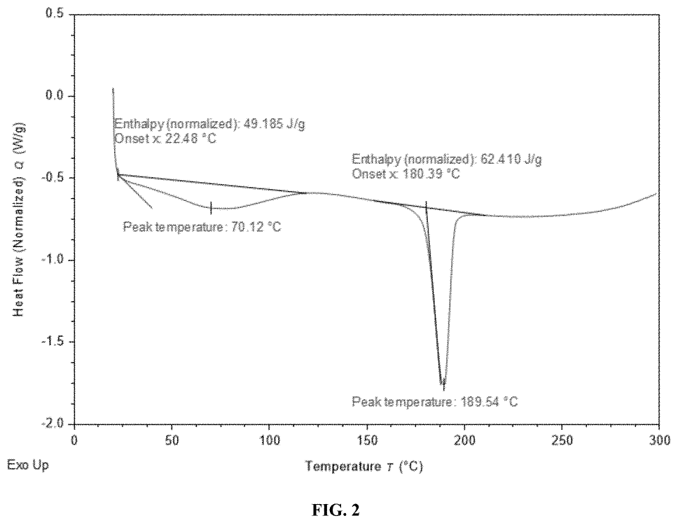

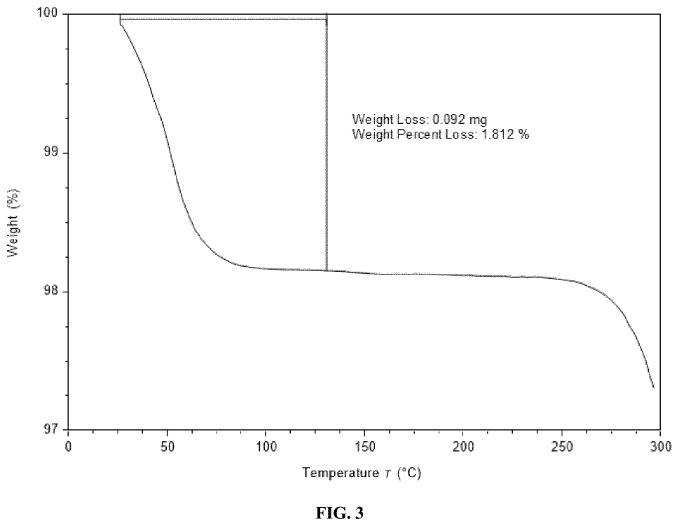

In some embodiments, Form I exhibits a DSC thermogram having endotherm peaks at temperatures of about 70° C. and about 190° C. In some embodiments, Form I exhibits a DSC thermogram having an endotherm peak at a temperature of about 70° C. In some embodiments, Form I exhibits a DSC thermogram having an endotherm peak at a temperature of about 190° C. In some embodiments, Form I has a DSC thermogram substantially as depicted in FIG. 2. In some embodiments, Form I has a TGA thermogram substantially as depicted in FIG. 3.

In some embodiments, Form I has at least one characteristic XRPD peak selected from about 4.9, about 9.3, about 12.3, about 14.7, and about 16.3 degrees 2-theta; and Form I exhibits a DSC thermogram having endotherm peaks at temperatures of about 70° C. and about 190° C. In some embodiments, Form I has at least one characteristic XRPD peak selected from about 4.9, about 9.3, about 12.3, about 14.7, and about 16.3 degrees 2-theta; and Form I exhibits a DSC thermogram having an endotherm peak at a temperatures of about 70° C. In some embodiments, Form I has at least one characteristic XRPD peak selected from about 4.9, about 9.3, about 12.3, about 14.7, and about 16.3 degrees 2-theta; and Form I exhibits a DSC thermogram having an endotherm peak at a temperatures of about 190° C.

In some embodiments, Form I can be isolated with a purity of at least about 80%, about 85%, about 90%, about 95%, about 96%, about 97%, about 98%, or about 99%. In some embodiments, Form I can be isolated with a purity greater than about 99%.

### Compound 1 Form II

Provided herein is a solid form of Compound 1 which is crystalline, referred to as Form II, or Compound 1 Form II, which is described below in the Examples. The data characterizing Form II is consistent with a methanol solvate.

Provided herein are also processes for preparing Form II comprising recrystallizing Compound 1 in a recrystallizing solvent. In some embodiments, the recrystallizing comprises precipitating Compound 1 Form II from a mixture of Compound 1 and the recrystallizing solvent. In some embodiments, the recrystallizing solvent is a polar solvent. In some embodiments, the solvent is C₁₋₆ alkyl alcohol solvent. In some embodiments, the solvent is methanol. In some embodiments, the precipitating is performed at an elevated temperature. In some embodiments, the elevated temperature is from about 30° C. to about 50° C. In some embodiments, the elevated temperature is about 40° C.

In some embodiments, Form II has at least one characteristic XRPD peak selected from about 7.4, about 12.7, about 13.6, about 20.8, and about 23.2 degrees 2-theta. In some embodiments, Form II has at least two characteristic XRPD peaks selected from about 7.4, about 12.7, about 13.6, about 20.8, and about 23.2 degrees 2-theta. In some embodiments, Form II has at least three characteristic XRPD peaks selected from about 7.4, about 12.7, about 13.6, about 20.8, and about 23.2 degrees 2-theta. In some embodiments, Form II has at least four characteristic XRPD peaks selected from about 7.4, about 12.7, about 13.6, about 20.8, and about 23.2 degrees 2-theta. In some embodiments, Form II has at least five characteristic XRPD peaks selected from about 7.4, about 12.7, about 13.6, about 20.8, and about 23.2 degrees 2-theta. In some embodiments, Form II has characteristic XRPD peaks at about 7.4, about 12.7, about 13.6, about 20.8, and about 23.2. In some embodiments, Form II has a characteristic XRPD peak at about 7.4 degrees 2-theta. In some embodiments, Form II has a characteristic XRPD peak at about 12.7 degrees 2-theta. In some embodiments, Form II has a characteristic XRPD peak at about 13.6 degrees 2-theta. In some embodiments, Form II has a characteristic XRPD peak at about 20.8 degrees 2-theta. In some embodiments, Form II has a characteristic XRPD peak at about 23.2 degrees 2-theta.

In some embodiments, Form II has at least one characteristic XRPD peak selected from about 7.4, about 12.5, about 12.7, about 13.6, about 14.5, about 15.7, about 16.9, about 20.8, about 23.2, and about 25.9 degrees 2-theta. In some embodiments, Form II has at least two characteristic XRPD peaks selected from about 7.4, about 12.5, about 12.7, about 13.6, about 14.5, about 15.7, about 16.9, about 20.8, about 23.2, and about 25.9 degrees 2-theta. In some embodiments, Form II has at least three characteristic XRPD peaks selected from about 7.4, about 12.5, about 12.7, about 13.6, about 14.5, about 15.7, about 16.9, about 20.8, about 23.2, and about 25.9 degrees 2-theta. In some embodiments, Form II has at least four characteristic XRPD peaks selected from about 7.4, about 12.5, about 12.7, about 13.6, about 14.5, about 15.7, about 16.9, about 20.8, about 23.2, and about 25.9 degrees 2-theta. In some embodiments, Form II has at least five characteristic XRPD peaks selected from about 7.4, about 12.5, about 12.7, about 13.6, about 14.5, about 15.7, about 16.9, about 20.8, about 23.2, and about 25.9 degrees 2-theta. In some embodiments, Form II has characteristic XRPD peaks at about 7.4, about 12.5, about 12.7, about 13.6, about 14.5, about 15.7, about 16.9, about 20.8, about 23.2, and about 25.9 degrees 2-theta.

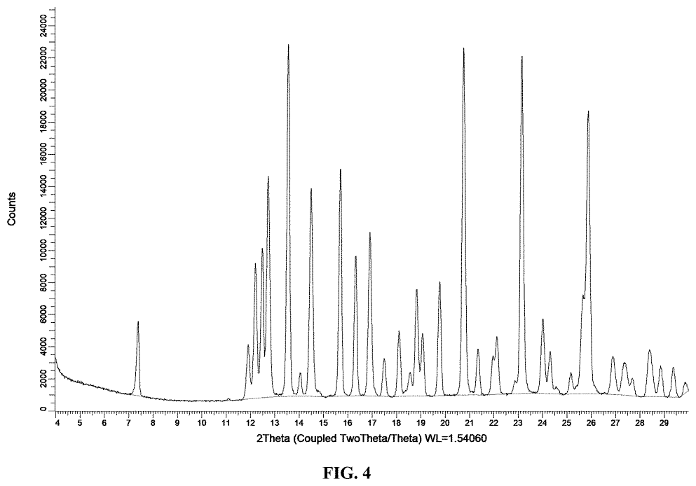

In some embodiments, Form II has an XRPD pattern with characteristic peaks as substantially shown in FIG. 4.

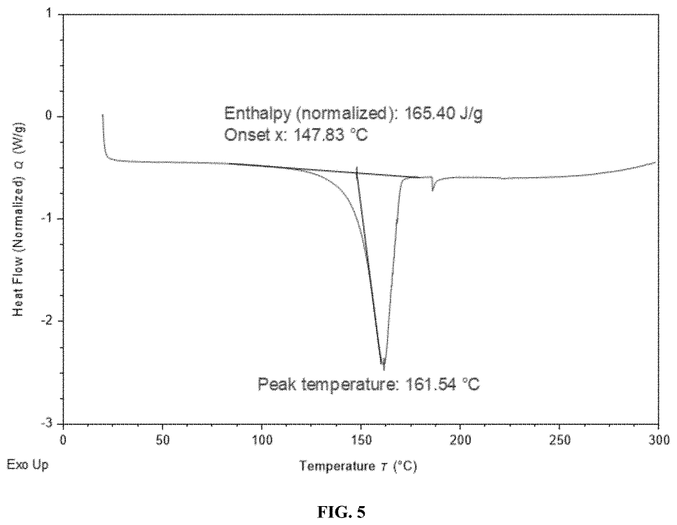

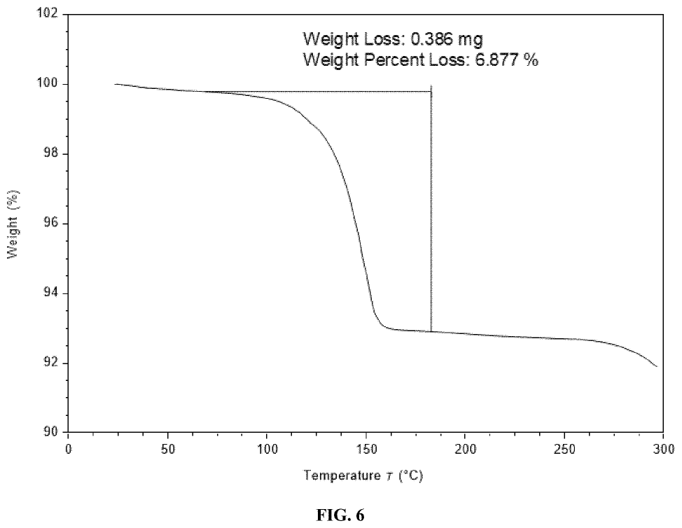

In some embodiments, Form II exhibits a DSC thermogram having an endotherm peak at a temperature of about 162° C. In some embodiments, Form II has a DSC thermogram substantially as depicted in FIG. 5. In some embodiments, Form II has a TGA thermogram substantially as depicted in FIG. 6.

In some embodiments, Form II has at least one characteristic XRPD peak selected from about 7.4, about 12.7, about 13.6, about 20.8, and about 23.2 degrees 2-theta; and Form II exhibits a DSC thermogram having an endotherm peak at a temperatures of about 162° C.

In some embodiments, Form II can be isolated with a purity of at least about 80%, about 85%, about 90%, about 95%, about 96%, about 97%, about 98%, or about 99%. In some embodiments, Form II can be isolated with a purity greater than about 99%.

### Phosphoric Acid Salts

Compound 1 phosphate can be prepared by any suitable method for preparation of phosphoric acid addition salts. For example, Compound 1 can be combined with phosphoric acid (e.g., about 1.0 molar eq or more) in a solvent and the resulting salt can be isolated by filtering the salt from solution. In certain embodiments, Compound 1 is combined with about 1 to about 2 molar equivalents of phosphoric acid. In certain embodiments, Compound 1 is combined with about 1 to about 1.5 molar equivalents of phosphoric acid. In certain embodiments, Compound 1 is combined with about 1.05 molar equivalents of phosphoric acid. In some embodiments, the solvent is a polar solvent. In some embodiments, the solvent is a protic solvent. In some embodiments, the solvent is a polar aprotic solvent. In some embodiments, the solvent is a C₁₋₆ alkyl alcohol. In some embodiments, the solvent is methanol. In some embodiments, the solvent is ethanol. In some embodiments, the solvent is a tetrahydrofuran. In some embodiments, the solvent is a mixture of a protic solvent and a polar aprotic solvent. In some embodiments the solvent is a mixture of methanol and tetrahydrofuran.

### Compound 1 Phosphate Form I

Provided herein is a solid form of Compound 1 phosphate which is crystalline, referred to as Compound 1 phosphate Form I, which is described below in the Examples.

Provided herein are also processes for preparing Compound 1 phosphate Form I comprising recrystallizing Compound 1 phosphate in a recrystallizing solvent. In some embodiments, the recrystallizing comprises precipitating Compound 1 phosphate Form I from a mixture of Compound 1 phosphate and the recrystallizing solvent. In some embodiments, the recrystallizing solvent is a polar solvent. In some embodiments, the recrystallizing solvent is a protic solvent. In some embodiments, the recrystallizing solvent is a polar aprotic solvent. In some embodiments, the crystallizing solvent is a C₁₋₆ alkyl alcohol. In some embodiments, the recrystallizing solvent is methanol. In some embodiments, the recrystallizing solvent is ethanol. In some embodiments, the recrystallizing solvent is tetrahydrofuran. In some embodiments, the recrystallizing solvent is a mixture of a protic solvent and a polar aprotic solvent. In some embodiments, the recrystallizing solvent is a mixture of methanol and tetrahydrofuran. In some embodiments, the precipitating is performed at a reduced temperature. In some embodiments, the reduced temperature is from about 10° C. to about 30° C. In some embodiments, the reduced temperature is about room temperature.

In some embodiments, Compound 1 phosphate Form I has at least one characteristic XRPD peak selected from about 8.2, about 9.6, about 13.8, about 15.0, and about 22.6 degrees 2-theta. In some embodiments, Compound 1 phosphate Form I has at least two characteristic XRPD peaks selected from about 8.2, about 9.6, about 13.8, about 15.0, and about 22.6 degrees 2-theta. In some embodiments, Compound 1 phosphate Form I has at least three characteristic XRPD peaks selected from about 8.2, about 9.6, about 13.8, about 15.0, and about 22.6 degrees 2-theta. In some embodiments, Compound 1 phosphate Form I has at least four characteristic XRPD peaks selected from about 8.2, about 9.6, about 13.8, about 15.0, and about 22.6 degrees 2-theta. In some embodiments, Compound 1 phosphate Form I has at least five characteristic XRPD peaks selected from about 8.2, about 9.6, about 13.8, about 15.0, and about 22.6 degrees 2-theta. In some embodiments, Compound 1 phosphate Form I has characteristic XRPD peaks at about 8.2, about 9.6, about 13.8, about 15.0, and about 22.6 degrees 2-theta. In some embodiments, Compound 1 phosphate Form I has a characteristic XRPD peak at about 8.2 degrees 2-theta. In some embodiments, Compound 1 phosphate Form I has a characteristic XRPD peak at about 9.6 degrees 2-theta. In some embodiments, Compound 1 phosphate Form I has a characteristic XRPD peak at about 13.8 degrees 2-theta. In some embodiments, Compound 1 phosphate Form I has a characteristic XRPD peak at about 15.0 degrees 2-theta. In some embodiments, Compound 1 phosphate Form I has a characteristic XRPD peak at about 22.6 degrees 2-theta.

In some embodiments, Compound 1 phosphate Form I has at least one characteristic XRPD peak selected from about 8.2, about 9.6, about 13.8, about 15.0, about 16.1, about 16.6, about 18.4, about 19.3, about 20.1, and about 22.6 degrees 2-theta. In some embodiments, Compound 1 phosphate Form I has at least two characteristic XRPD peaks selected from about 8.2, about 9.6, about 13.8, about 15.0, about 16.1, about 16.6, about 18.4, about 19.3, about 20.1, and about 22.6 degrees 2-theta. In some embodiments, Compound 1 phosphate Form I has at least three characteristic XRPD peaks selected from about 8.2, about 9.6, about 13.8, about 15.0, about 16.1, about 16.6, about 18.4, about 19.3, about 20.1, and about 22.6 degrees 2-theta. In some embodiments, Compound 1 phosphate Form I has at least four characteristic XRPD peaks selected from about 8.2, about 9.6, about 13.8, about 15.0, about 16.1, about 16.6, about 18.4, about 19.3, about 20.1, and about 22.6 degrees 2-theta. In some embodiments, Compound 1 phosphate Form I has at least five characteristic XRPD peaks selected from about 8.2, about 9.6, about 13.8, about 15.0, about 16.1, about 16.6, about 18.4, about 19.3, about 20.1, and about 22.6 degrees 2-theta. In some embodiments, Compound 1 phosphate Form I has characteristic XRPD peaks at about 8.2, about 9.6, about 13.8, about 15.0, about 16.1, about 16.6, about 18.4, about 19.3, about 20.1, and about 22.6 degrees 2-theta.

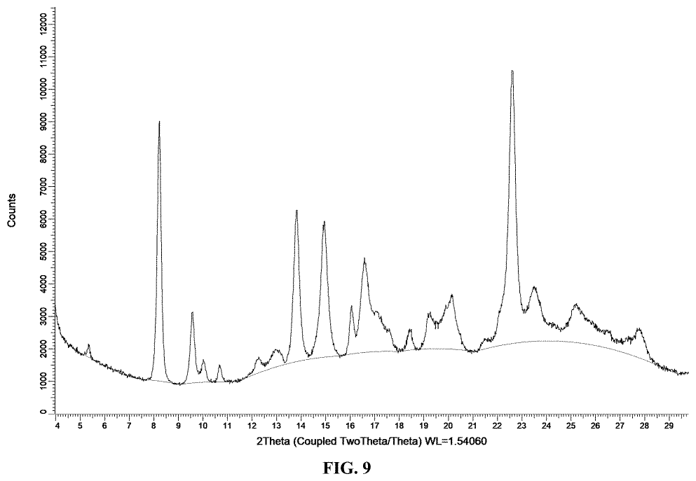

In some embodiments, Compound 1 phosphate Form I has an XRPD pattern with characteristic peaks as substantially shown in FIG. 9.

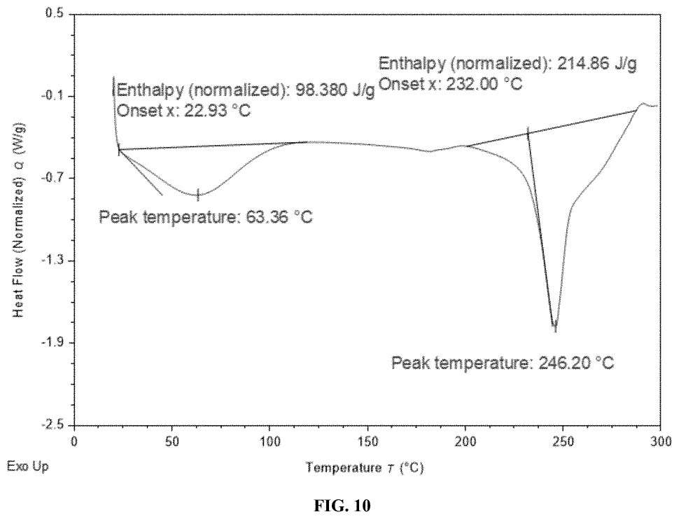

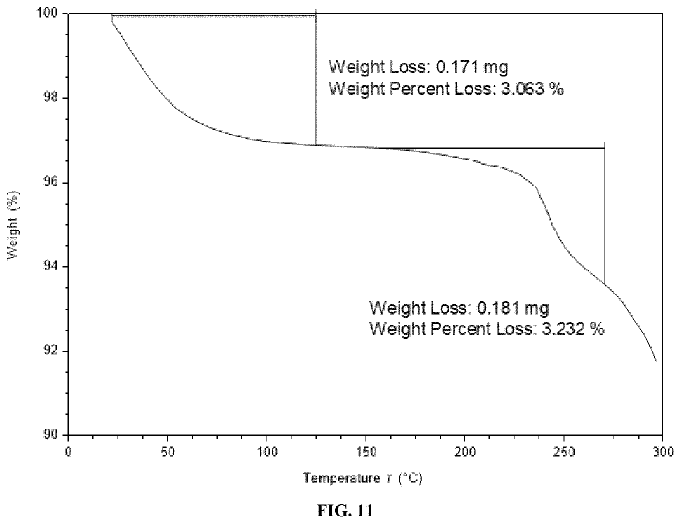

In some embodiments, Compound 1 phosphate Form I exhibits a DSC thermogram having endotherm peaks at temperatures of about 63° C. and about 246° C. In some embodiments, Compound 1 phosphate Form I exhibits a DSC thermogram having an endotherm peak at a temperature of about 63° C. In some embodiments, Compound 1 phosphate Form I exhibits a DSC thermogram having an endotherm peak at a temperature of about 246° C. In some embodiments, Compound 1 phosphate Form I has a DSC thermogram substantially as depicted in FIG. 10. In some embodiments, Compound 1 phosphate Form I has a TGA thermogram substantially as depicted in FIG. 11.

In some embodiments, Compound 1 phosphate Form I has at least one characteristic XRPD peak selected from about 8.2, about 9.6, about 13.8, about 15.0 and about 22.6 degrees 2-theta; and Compound 1 phosphate Form I exhibits a DSC thermogram having endotherm peaks at temperatures of about 63° C. and about 246° C. In some embodiments, Compound 1 phosphate Form I has at least one characteristic XRPD peak selected from about 8.2, about 9.6, about 13.8, about 15.0 and about 22.6 degrees 2-theta; and Compound 1 phosphate Form I exhibits a DSC thermogram having an endotherm peak at a temperatures of about 63° C. In some embodiments, Compound 1 phosphate Form I has at least one characteristic XRPD peak selected from about 8.2, about 9.6, about 13.8, about 15.0 and about 22.6 degrees 2-theta; and Compound 1 phosphate Form I exhibits a DSC thermogram having an endotherm peak at a temperatures of about 246° C.

In some embodiments, Compound 1 phosphate Form I can be isolated with a purity of at least about 80%, about 85%, about 90%, about 95%, about 96%, about 97%, about 98%, or about 99%. In some embodiments, Compound 1 phosphate Form I can be isolated with a purity greater than about 99%.

### Compound 1 Phosphate Form II

Provided herein is a solid form of Compound 1 phosphate which is crystalline, referred to as Compound 1 Phosphate Form II, which is described below in the Examples.

Provided herein are also processes for preparing Compound 1 phosphate Form II comprising recrystallizing Compound 1 phosphate in a recrystallizing solvent. In some embodiments, the recrystallizing comprises precipitating Compound 1 phosphate Form II from a mixture of Compound 1 phosphate and the recrystallizing solvent. In some embodiments, the recrystallizing solvent is a polar solvent. In some embodiments, the recrystallizing solvent is a polar aprotic solvent. In some embodiments, the recrystallizing solvent is dimethylformamide. In some embodiments, the recrystallizing solvent is tetrahydrofuran. In some embodiments, the recrystallizing solvent is dimethylsulfoxide. In some embodiments, the precipitating is performed at an elevated temperature. In some embodiments, the elevated temperature is from about 40° C. to about 60° C. In some embodiments, the elevated temperature is about 50° C. In some embodiments, the elevated temperature is from about 15° C. to about 35° C. In some embodiments, the elevated temperature is about 25° C.

In some embodiments, Compound 1 phosphate Form II has at least one characteristic XRPD peak selected from about 3.9, about 6.9, about 12.9, about 18.3, and about 23.5 degrees 2-theta. In some embodiments, Compound 1 phosphate Form II has at least two characteristic XRPD peaks selected from about 3.9, about 6.9, about 12.9, about 18.3, and about 23.5 degrees 2-theta. In some embodiments, Compound 1 phosphate Form II has at least three characteristic XRPD peaks selected from about 3.9, about 6.9, about 12.9, about 18.3, and about 23.5 degrees 2-theta. In some embodiments, Compound 1 phosphate Form II has at least four characteristic XRPD peaks selected from about 3.9, about 6.9, about 12.9, about 18.3, and about 23.5 degrees 2-theta. In some embodiments, Compound 1 phosphate Form II has at least five characteristic XRPD peaks selected from about 3.9, about 6.9, about 12.9, about 18.3, and about 23.5 degrees 2-theta. In some embodiments, Compound 1 phosphate Form II has characteristic XRPD peaks at about 3.9, about 6.9, about 12.9, about 18.3, and about 23.5 degrees 2-theta. In some embodiments, Compound 1 phosphate Form II has a characteristic XRPD peak at about 3.9 degrees 2-theta. In some embodiments, Compound 1 phosphate Form II has a characteristic XRPD peak at about 6.9 degrees 2-theta. In some embodiments, Compound 1 phosphate Form II has a characteristic XRPD peak at about 12.9 degrees 2-theta. In some embodiments, Compound 1 phosphate Form II has a characteristic XRPD peak at about 18.3 degrees 2-theta. In some embodiments, Compound 1 phosphate Form II has a characteristic XRPD peak at about 23.5 degrees 2-theta.

In some embodiments, Compound 1 phosphate Form II has at least one characteristic XRPD peak selected from about 3.9, about 6.9, about 11.6, about 12.9, about 15.6, about 16.9, about 18.3, about 23.5, and about 26.8 degrees 2-theta. In some embodiments, Compound 1 phosphate Form II has at least two characteristic XRPD peaks selected from about 3.9, about 6.9, about 11.6, about 12.9, about 15.6, about 16.9, about 18.3, about 23.5, and about 26.8 degrees 2-theta. In some embodiments, Compound 1 phosphate Form II has at least three characteristic XRPD peaks selected from about 3.9, about 6.9, about 11.6, about 12.9, about 15.6, about 16.9, about 18.3, about 23.5 and about 26.8 degrees 2-theta. In some embodiments, Compound 1 phosphate Form II has at least four characteristic XRPD peaks selected from about 3.9, about 6.9, about 11.6, about 12.9, about 15.6, about 16.9, about 18.3, about 23.5 and about 26.8 degrees 2-theta. In some embodiments, Compound 1 phosphate Form II has at least five characteristic XRPD peaks selected from about 3.9, about 6.9, about 11.6, about 12.9, about 15.6, about 16.9, about 18.3, about 23.5 and about 26.8 degrees 2-theta. In some embodiments, Compound 1 phosphate Form II has characteristic XRPD peaks at about 3.9, about 6.9, about 11.6, about 12.9, about 15.6, about 16.9, about 18.3, about 23.5 and about 26.8 degrees 2-theta.

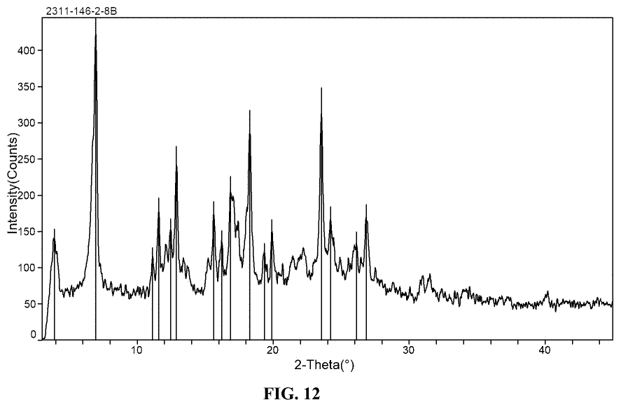

In some embodiments, Compound 1 phosphate Form II has an XRPD pattern with characteristic peaks as substantially shown in FIG. 12.

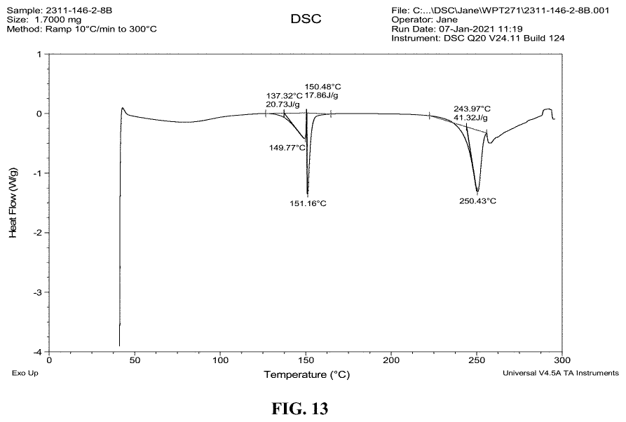

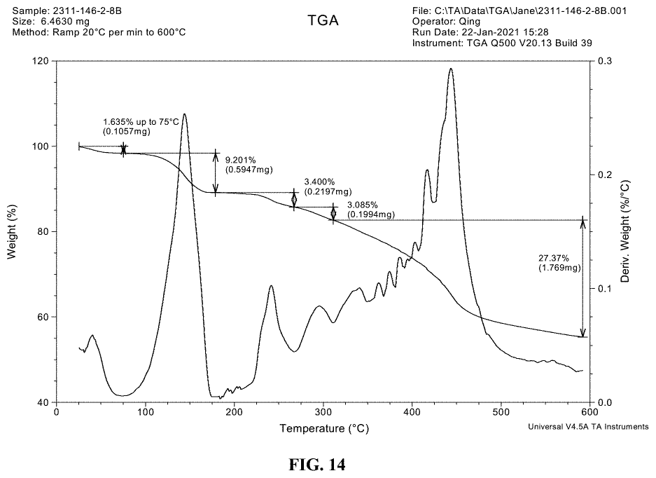

In some embodiments, Compound 1 phosphate Form II exhibits a DSC thermogram having endotherm peaks at temperatures of about 151° C. and about 250° C. In some embodiments, Compound 1 phosphate Form II exhibits a DSC thermogram having an endotherm peak at a temperature of about 151° C. In some embodiments, Compound 1 phosphate Form II exhibits a DSC thermogram having an endotherm peak at a temperature of about 250° C. In some embodiments, Compound 1 phosphate Form II has a DSC thermogram substantially as depicted in FIG. 13. In some embodiments, Compound 1 phosphate Form II has a TGA thermogram substantially as depicted in FIG. 14.

In some embodiments, Compound 1 phosphate Form II has at least one characteristic XRPD peak selected from about 3.9, about 6.9, about 12.9, about 18.3, and about 23.5 degrees 2-theta; and Compound 1 phosphate Form II exhibits a DSC thermogram having endotherm peaks at temperatures of about 151° C. and about 250° C. In some embodiments, Compound 1 phosphate Form II has at least one characteristic XRPD peak selected from about 3.9, about 6.9, about 12.9, about 18.3, and about 23.5 degrees 2-theta; and Compound 1 phosphate Form II exhibits a DSC thermogram having an endotherm peak at a temperatures of about 151° C. In some embodiments, Compound 1 phosphate Form II has at least one characteristic XRPD peak selected from about 3.9, about 6.9, about 12.9, about 18.3, and about 23.5 degrees 2-theta; and Compound 1 phosphate Form II exhibits a DSC thermogram having an endotherm peak at a temperatures of about 250° C.

In some embodiments, Compound 1 phosphate Form II can be isolated with a purity of at least about 80%, about 85%, about 90%, about 95%, about 96%, about 97%, about 98%, or about 99%. In some embodiments, Compound 1 phosphate Form II can be isolated with a purity greater than about 99%.

### Compound 1 Phosphate Form III

Provided herein is a solid form of Compound 1 phosphate which is crystalline, referred to as Compound 1 phosphate Form III, which is described below in the Examples. The data characterizing Compound 1 phosphate Form III is consistent with an acetonitrile solvate.

Provided herein are also processes for preparing Compound 1 phosphate Form III comprising recrystallizing Compound 1 phosphate in a recrystallizing solvent. In some embodiments, the recrystallizing comprises precipitating Compound 1 phosphate Form III from a mixture of Compound 1 phosphate and the recrystallizing solvent. In some embodiments, the recrystallizing solvent is a polar solvent. In some embodiments, the recrystallizing solvent is a polar aprotic solvent. In some embodiments, the recrystallizing solvent is acetonitrile. In some embodiments, the precipitating is performed at a reduced temperature. In some embodiments, the reduced temperature is from about 40° C. to about 60° C. In some embodiments, the reduced temperature is about 50° C.

In some embodiments, Compound 1 phosphate Form III has at least one characteristic XRPD peak selected from about 3.9, about 5.0, about 16.2, and about 22.5 degrees 2-theta. In some embodiments, Compound 1 phosphate Form III has at least two characteristic XRPD peaks selected from about 3.9, about 5.0, about 16.2, and about 22.5 degrees 2-theta. In some embodiments, Compound 1 phosphate Form III has at least three characteristic XRPD peaks selected from about 3.9, about 5.0, about 16.2, and about 22.5 degrees 2-theta. In some embodiments, Compound 1 phosphate Form III has characteristic XRPD peaks at about 3.9, about 5.0, about 16.2, and about 22.5 degrees 2-theta. In some embodiments, Compound 1 phosphate Form III has a characteristic XRPD peak at about 3.9 degrees 2-theta. In some embodiments, Compound 1 phosphate Form III has a characteristic XRPD peak at about 5.0 degrees 2-theta. In some embodiments, Compound 1 phosphate Form III has a characteristic XRPD peak at about 16.2 degrees 2-theta. In some embodiments, Compound 1 phosphate Form III has a characteristic XRPD peak at about 22.5 degrees 2-theta.

In some embodiments, Compound 1 phosphate Form III has at least one characteristic XRPD peak selected from about 3.9, about 5.0, about 5.7, about 8.1, about 12.4, about 14.0, about 16.2, about 17.0, about 20.3, and about 22.5 degrees 2-theta. In some embodiments, Compound 1 phosphate Form III has at least two characteristic XRPD peaks selected from about 3.9, about 5.0, about 5.7, about 8.1, about 12.4, about 14.0, about 16.2, about 17.0, about 20.3, and about 22.5 degrees 2-theta. In some embodiments, Compound 1 phosphate Form III has at least three characteristic XRPD peaks selected from about 3.9, about 5.0, about 5.7, about 8.1, about 12.4, about 14.0, about 16.2, about 17.0, about 20.3, and about 22.5 degrees 2-theta. In some embodiments, Compound 1 phosphate Form III has at least four characteristic XRPD peaks selected from about 3.9, about 5.0, about 5.7, about 8.1, about 12.4, about 14.0, about 16.2, about 17.0, about 20.3, and about 22.5 degrees 2-theta. In some embodiments, Compound 1 phosphate Form III has at least five characteristic XRPD peaks selected from about 3.9, about 5.0, about 5.7, about 8.1, about 12.4, about 14.0, about 16.2, about 17.0, about 20.3, and about 22.5 degrees 2-theta. In some embodiments, Compound 1 phosphate Form III has characteristic XRPD peaks at about 3.9, about 5.0, about 5.7, about 8.1, about 12.4, about 14.0, about 16.2, about 17.0, about 20.3, and about 22.5 degrees 2-theta.

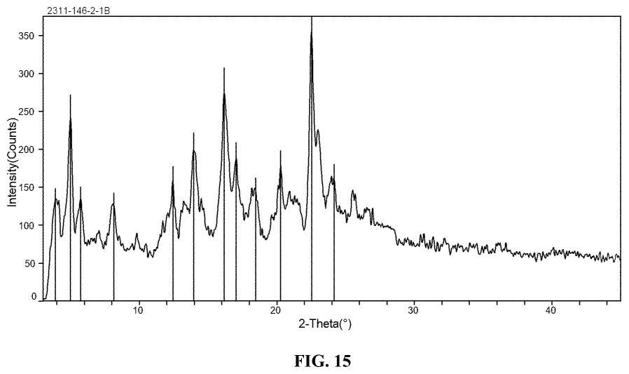

In some embodiments, Compound 1 phosphate Form III has an XRPD pattern with characteristic peaks as substantially shown in FIG. 15.

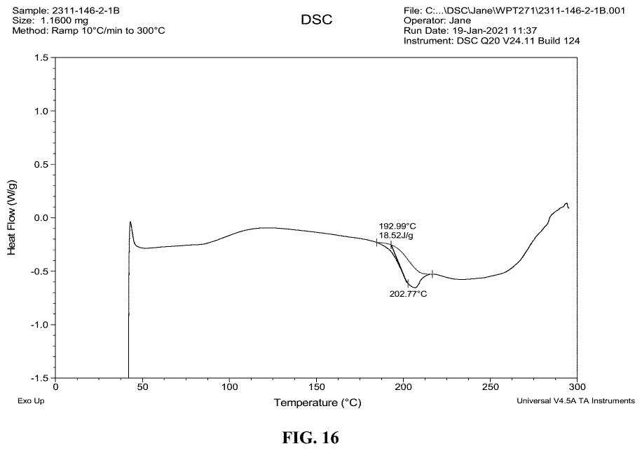

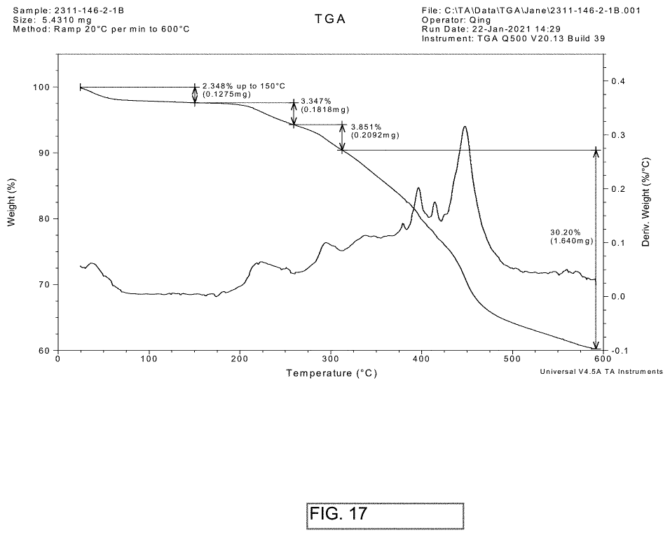

In some embodiments, Compound 1 phosphate Form III exhibits a DSC thermogram having an endotherm peak at a temperature of about 203° C. In some embodiments, Compound 1 phosphate Form III has a DSC thermogram substantially as depicted in FIG. 16. In some embodiments, Compound 1 phosphate Form III has a TGA thermogram substantially as depicted in FIG. 17.

In some embodiments, Compound 1 phosphate Form III has at least one characteristic XRPD peak selected from about 3.9, about 5.0, about 16.2, and about 22.5 degrees 2-theta; and Compound 1 phosphate Form III exhibits a DSC thermogram having an endotherm peak at a temperatures of about 203° C.

In some embodiments, Compound 1 phosphate Form III can be isolated with a purity of at least about 80%, about 85%, about 90%, about 95%, about 96%, about 97%, about 98%, or about 99%. In some embodiments, Compound 1 phosphate Form III can be isolated with a purity greater than about 99%.

### Hydrochloric Acid Salts

Compound 1 hydrochloride can be prepared by any suitable method for preparation of hydrochloric acid addition salts. For example, Compound 1 can be combined with hydrochloric acid (e.g., about 1.0 molar eq or more) in a solvent and the resulting salt can be isolated by filtering the salt from solution. In certain embodiments, Compound 1 is combined with about 1 to about 5 molar equivalents of hydrochloric acid. In certain embodiments, Compound 1 is combined with about 1 to about 2 molar equivalents of hydrochloric acid. In certain embodiments, Compound 1 is combined with about 1 to about 1.5 molar equivalents of hydrochloric acid. In certain embodiments, Compound 1 is combined with about 1.1 molar equivalents of hydrochloric acid. In certain embodiments, Compound 1 is combined with about 2.2 molar equivalents of hydrochloric acid. In some embodiments, the solvent is a polar solvent. In some embodiments, the solvent is a polar aprotic solvent. In some embodiments, the solvent is a protic solvent. In some embodiments, the solvent is a halogenated solvent. In some embodiments, the solvent is a chlorinated solvent. In some embodiments, the solvent is dichloromethane. In some embodiments, the solvent is a C₁₋₆ alkyl alcohol. In some embodiments, the solvent is methanol. In some embodiments, the solvent is a mixture of a polar aprotic solvent and a protic solvent. In some embodiments, the solvent is a mixture of a halogenated solvent and a C₁₋₆ alkyl alcohol. In some embodiments, the solvent is a mixture of dichloromethane and methanol.

### Compound 1 Hydrochloride Form I

Provided herein is a solid form of Compound 1 hydrochloride which is crystalline, referred to as Compound 1 hydrochloride Form I, which is described below in the Examples.

Provided herein are also processes for preparing Form I of Compound 1 hydrochloride comprising recrystallizing Compound 1 hydrochloride in a recrystallizing solvent. In some embodiments, the recrystallizing comprises precipitating Compound 1 hydrochloride Form I from a mixture of Compound 1 hydrochloride and the recrystallizing solvent. In some embodiments, the recrystallizing solvent is a polar solvent. In some embodiments, the recrystallizing solvent is a polar aprotic solvent. In some embodiments, the recrystallizing solvent is acetone. In some embodiments, the precipitating is performed at an elevated temperature. In some embodiments, the elevated temperature is from about 55° C. to about 80° C. In some embodiments, the reduced temperature is from about 65° C. to about 70° C.

In some embodiments, Compound 1 hydrochloride Form I has at least one characteristic XRPD peak selected from about 5.0, about 6.4, about 7.8, about 10.1, about 15.1, and about 24.0 degrees 2-theta. In some embodiments, Compound 1 hydrochloride Form I has at least two characteristic XRPD peaks selected from about 5.0, about 6.4, about 7.8, about 10.1, about 15.1, and about 24.0 degrees 2-theta. In some embodiments, Compound 1 hydrochloride Form I has at least three characteristic XRPD peaks selected from about 5.0, about 6.4, about 7.8, about 10.1, about 15.1, and about 24.0 degrees 2-theta. In some embodiments, Compound 1 hydrochloride Form I has at least four characteristic XRPD peaks selected from about 5.0, about 6.4, about 7.8, about 10.1, about 15.1, and about 24.0 degrees 2-theta. In some embodiments, Compound 1 hydrochloride Form I has at least five characteristic XRPD peaks selected from about 5.0, about 6.4, about 7.8, about 10.1, about 15.1, and about 24.0 degrees 2-theta. In some embodiments, Compound 1 hydrochloride Form I has characteristic XRPD peaks at about 5.0, about 6.4, about 7.8, about 10.1, about 15.1, and about 24.0 degrees 2-theta. In some embodiments, Compound 1 hydrochloride Form I has a characteristic XRPD peak at about 5.0 degrees 2-theta. In some embodiments, Compound 1 hydrochloride Form I has a characteristic XRPD peak at about 6.4 degrees 2-theta. In some embodiments, Compound 1 hydrochloride Form I has a characteristic XRPD peak at about 7.8 degrees 2-theta. In some embodiments, Compound 1 hydrochloride Form I has a characteristic XRPD peak at about 10.1 degrees 2-theta. In some embodiments, Compound 1 hydrochloride Form I has a characteristic XRPD peak at about 15.1 degrees 2-theta. In some embodiments, Compound 1 hydrochloride Form I has a characteristic XRPD peak at about 24.0 degrees 2-theta.

In some embodiments, Compound 1 hydrochloride Form I has at least one characteristic XRPD peak selected from about 5.0, about 6.4, about 7.8, about 10.1, about 15.1, about 15.7, about 19.8, about 21.0, about 24.0, about 25.2, about 26.2, and about 26.4 degrees 2-theta. In some embodiments, Compound 1 hydrochloride Form I has at least two characteristic XRPD peaks selected from about 5.0, about 6.4, about 7.8, about 10.1, about 15.1, about 15.7, about 19.8, about 21.0, about 24.0, about 25.2, about 26.2, and about 26.4 degrees 2-theta. In some embodiments, Compound 1 hydrochloride Form I has at least three characteristic XRPD peaks selected from about 5.0, about 6.4, about 7.8, about 10.1, about 15.1, about 15.7, about 19.8, about 21.0, about 24.0, about 25.2, about 26.2, and about 26.4 degrees 2-theta. In some embodiments, Compound 1 hydrochloride Form I has at least four characteristic XRPD peaks selected from about 5.0, about 6.4, about 7.8, about 10.1, about 15.1, about 15.7, about 19.8, about 21.0, about 24.0, about 25.2, about 26.2, and about 26.4 degrees 2-theta. In some embodiments, Compound 1 hydrochloride Form I has at least five characteristic XRPD peaks selected from about 5.0, about 6.4, about 7.8, about 10.1, about 15.1, about 15.7, about 19.8, about 21.0, about 24.0, about 25.2, about 26.2, and about 26.4 degrees 2-theta. In some embodiments, Compound 1 hydrochloride Form I has characteristic XRPD peaks at about 5.0, about 6.4, about 7.8, about 10.1, about 15.1, about 15.7, about 19.8, about 21.0, about 24.0, about 25.2, about 26.2, and about 26.4 degrees 2-theta.

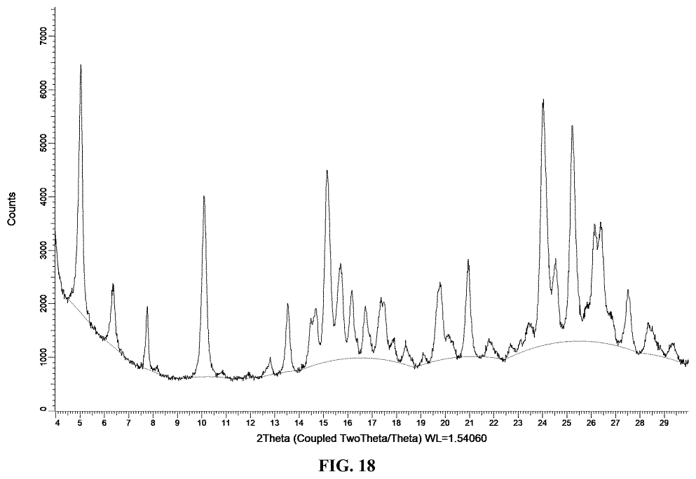

In some embodiments, Compound 1 hydrochloride Form I has an XRPD pattern with characteristic peaks as substantially shown in FIG. 18.

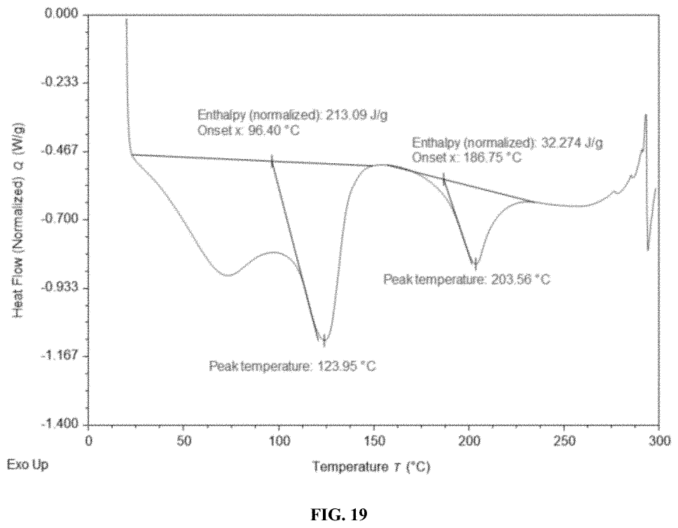

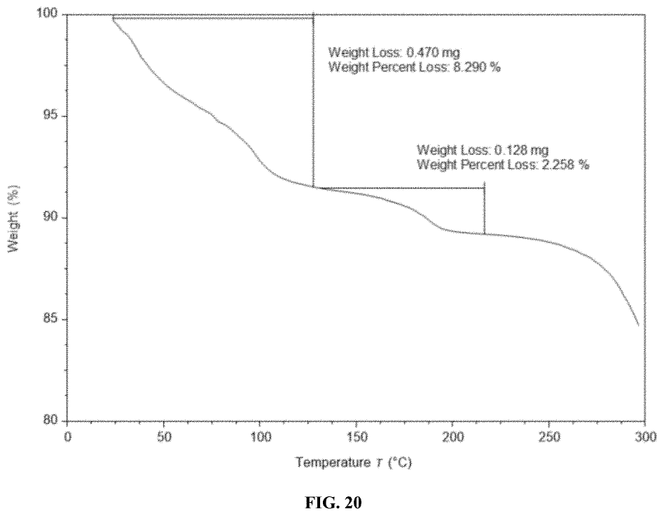

In some embodiments, Compound 1 hydrochloride Form I exhibits a DSC thermogram having endotherm peaks at temperatures of about 124° C. and about 204° C. In some embodiments, Compound 1 hydrochloride Form I exhibits a DSC thermogram having an endotherm peak at a temperature of about 124° C. In some embodiments, Compound 1 hydrochloride Form I exhibits a DSC thermogram having an endotherm peak at a temperature of about 204° C. In some embodiments, Compound 1 hydrochloride Form I has a DSC thermogram substantially as depicted in FIG. 19. In some embodiments, Compound 1 hydrochloride Form I has a TGA thermogram substantially as depicted in FIG. 20.

In some embodiments, Compound 1 hydrochloride Form I has at least one characteristic XRPD peak selected from about 5.0, about 6.4, about 7.8, about 10.1, about 15.1, and about 24.0 degrees 2-theta; and Compound 1 hydrochloride Form I exhibits a DSC thermogram having endotherm peaks at temperatures of about 124° C. and about 204° C. In some embodiments, Compound 1 hydrochloride Form I has at least one characteristic XRPD peak selected from about 5.0, about 6.4, about 7.8, about 10.1, about 15.1, and about 24.0 degrees 2-theta; and Compound 1 hydrochloride Form I exhibits a DSC thermogram having an endotherm peak at a temperatures of about 124° C. In some embodiments, Compound 1 hydrochloride Form I has at least one characteristic XRPD peak selected from about 5.0, about 6.4, about 7.8, about 10.1, about 15.1, and about 24.0 degrees 2-theta; and Compound 1 hydrochloride Form I exhibits a DSC thermogram having an endotherm peak at a temperatures of about 204° C.

In some embodiments, Compound 1 hydrochloride Form I can be isolated with a purity of at least about 80%, about 85%, about 90%, about 95%, about 96%, about 97%, about 98%, or about 99%. In some embodiments, Compound 1 hydrochloride Form I can be isolated with a purity greater than about 99%.

### Compound 1 Hydrochloride Form II

Provided herein is a solid form of Compound 1 hydrochloride which is crystalline, referred to as Compound 1 hydrochloride Form II, which is described below in the Examples.

Provided herein are also processes for preparing Compound 1 hydrochloride Form II comprising recrystallizing Compound 1 hydrochloride in a recrystallizing solvent. In some embodiments, the recrystallizing comprises precipitating Compound 1 hydrochloride Form II from a mixture of Compound 1 hydrochloride and the recrystallizing solvent. In some embodiments, the recrystallizing solvent is a polar solvent. In some embodiments, the recrystallizing solvent is a polar aprotic solvent. In some embodiments, the recrystallizing solvent is acetone. In some embodiments, the precipitating is performed at an elevated temperature. In some embodiments, the elevated temperature is from about 55° C. to about 80° C. In some embodiments, the elevated temperature is from about 65° C. to about 70° C.

In some embodiments, Compound 1 hydrochloride Form II has at least one characteristic XRPD peak selected from about 4.4, about 6.6, about 7.0, about 9.0, about 11.1, and about 13.8 degrees 2-theta. In some embodiments, Compound 1 hydrochloride Form II has at least two characteristic XRPD peaks selected from 4.4, about 6.6, about 7.0, about 9.0, about 11.1, and about 13.8 degrees 2-theta. In some embodiments, Compound 1 hydrochloride Form II has at least three characteristic XRPD peaks selected from about 4.4, about 6.6, about 7.0, about 9.0, about 11.1, and about 13.8 degrees 2-theta. In some embodiments, Compound 1 hydrochloride Form II has at least four characteristic XRPD peaks selected from about 4.4, about 6.6, about 7.0, about 9.0, about 11.1, and about 13.8 degrees 2-theta. In some embodiments, Compound 1 hydrochloride Form II has at least five characteristic XRPD peaks selected from about 4.4, about 6.6, about 7.0, about 9.0, about 11.1, and about 13.8 degrees 2-theta. In some embodiments, Compound 1 hydrochloride Form II has characteristic XRPD peaks at about 4.4, about 6.6, about 7.0, about 9.0, about 11.1, and about 13.8 degrees 2-theta. In some embodiments, Compound 1 hydrochloride Form II has a characteristic XRPD peak at about 4.4 degrees 2-theta. In some embodiments, Compound 1 hydrochloride Form II has a characteristic XRPD peak at about 6.6 degrees 2-theta. In some embodiments, Compound 1 hydrochloride Form II has a characteristic XRPD peak at about 7.0 degrees 2-theta. In some embodiments, Compound 1 hydrochloride Form II has a characteristic XRPD peak at about 9.0 degrees 2-theta. In some embodiments, Compound 1 hydrochloride Form II has a characteristic XRPD peak at about 11.1 degrees 2-theta. In some embodiments, Compound 1 hydrochloride Form II has a characteristic XRPD peak at about 13.8 degrees 2-theta.

In some embodiments, Compound 1 hydrochloride Form II has at least one characteristic XRPD peak selected from about 4.4, about 6.6, about 7.0, about 9.0, about 11.1, about 13.8, about 14.8, about 15.3, about 18.1, about 23.7, about 24.8, about 25.7, and about 26.2 degrees 2-theta. In some embodiments, Compound 1 hydrochloride Form II has at least two characteristic XRPD peaks selected from about 4.4, about 6.6, about 7.0, about 9.0, about 11.1, about 13.8, about 14.8, about 15.3, about 18.1, about 23.7, about 24.8, about 25.7, and about 26.2 degrees 2-theta. In some embodiments, Compound 1 hydrochloride Form II has at least three characteristic XRPD peaks selected from about 4.4, about 6.6, about 7.0, about 9.0, about 11.1, about 13.8, about 14.8, about 15.3, about 18.1, about 23.7, about 24.8, about 25.7, and about 26.2 degrees 2-theta. In some embodiments, Compound 1 hydrochloride Form II has at least four characteristic XRPD peaks selected from about 4.4, about 6.6, about 7.0, about 9.0, about 11.1, about 13.8, about 14.8, about 15.3, about 18.1, about 23.7, about 24.8, about 25.7, and about 26.2 degrees 2-theta. In some embodiments, Compound 1 hydrochloride Form II has at least five characteristic XRPD peaks selected from about 4.4, about 6.6, about 7.0, about 9.0, about 11.1, about 13.8, about 14.8, about 15.3, about 18.1, about 23.7, about 24.8, about 25.7, and about 26.2 degrees 2-theta. In some embodiments, Compound 1 hydrochloride Form II has characteristic XRPD peaks at about 4.4, about 6.6, about 7.0, about 9.0, about 11.1, about 13.8, about 14.8, about 15.3, about 18.1, about 23.7, about 24.8, about 25.7, and about 26.2 degrees 2-theta.

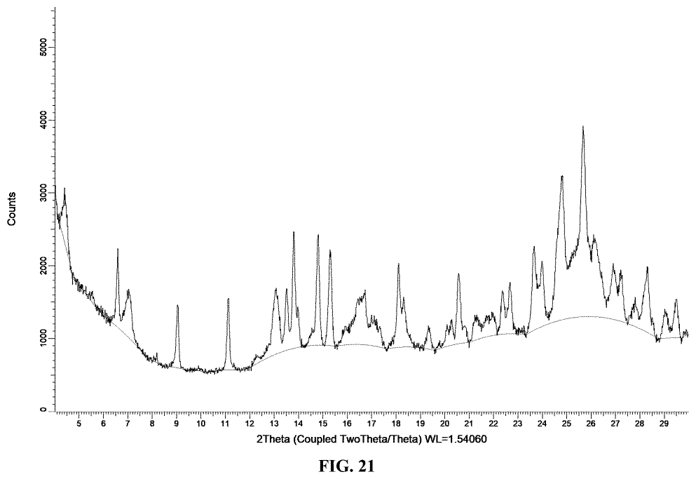

In some embodiments, Compound 1 hydrochloride Form II has an XRPD pattern with characteristic peaks as substantially shown in FIG. 21.

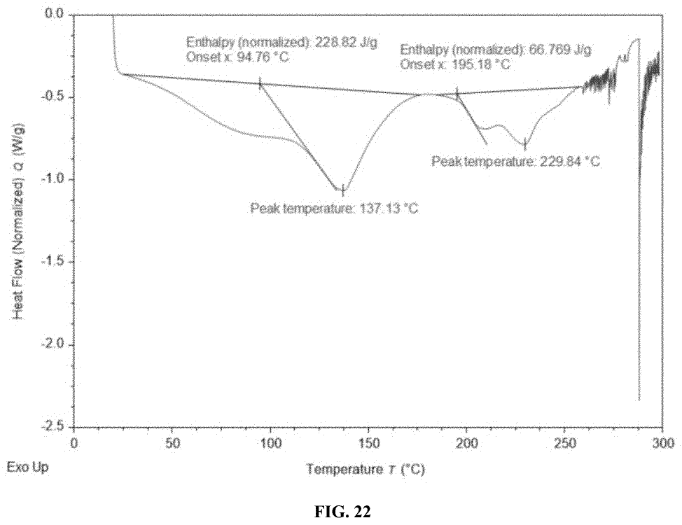

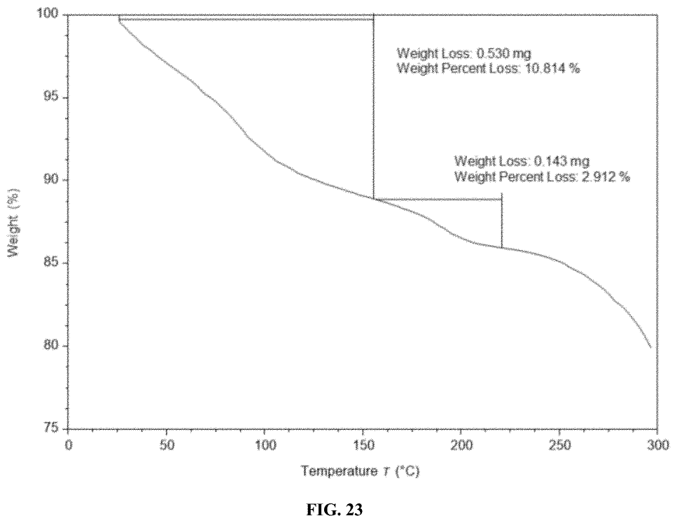

In some embodiments, Compound 1 hydrochloride Form II exhibits a DSC thermogram having endotherm peaks at temperatures of about 137° C. and about 230° C. In some embodiments, Compound 1 hydrochloride Form II exhibits a DSC thermogram having an endotherm peak at a temperature of about 137° C. In some embodiments, Compound 1 hydrochloride Form II exhibits a DSC thermogram having an endotherm peak at a temperature of about 230° C. In some embodiments, Compound 1 hydrochloride Form II has a DSC thermogram substantially as depicted in FIG. 22. In some embodiments, Compound 1 hydrochloride Form II has a TGA thermogram substantially as depicted in FIG. 23.

In some embodiments, Compound 1 hydrochloride Form II has at least one characteristic XRPD peak selected from about 4.4, about 6.6, about 7.0, about 9.0, about 11.1, and about 13.8 degrees 2-theta; and Compound 1 hydrochloride Form II exhibits a DSC thermogram having endotherm peaks at temperatures of about 137° C. and about 230° C. In some embodiments, Compound 1 hydrochloride Form II has at least one characteristic XRPD peak selected from about 4.4, about 6.6, about 7.0, about 9.0, about 11.1, and about 13.8 degrees 2-theta; and Compound 1 hydrochloride Form II exhibits a DSC thermogram having an endotherm peak at a temperatures of about 137° C. In some embodiments, Compound 1 hydrochloride Form II has at least one characteristic XRPD peak selected from about 4.4, about 6.6, about 7.0, about 9.0, about 11.1, and about 13.8 degrees 2-theta; and Compound 1 hydrochloride Form II exhibits a DSC thermogram having an endotherm peak at a temperatures of about 230° C.

In some embodiments, Compound 1 hydrochloride Form II can be isolated with a purity of at least about 80%, about 85%, about 90%, about 95%, about 96%, about 97%, about 98%, or about 99%. In some embodiments, Compound 1 hydrochloride Form II can be isolated with a purity greater than about 99%.

### L-(+)-Tartaric Acid Salts

Compound 1 L-(+)-tartrate can be prepared by any suitable method for preparation of L-(+)-tartaric acid addition salts. For example, Compound 1 can be combined with L-(+)-tartaric acid (e.g., about 1.0 molar eq or more) in a solvent and the resulting salt can be isolated by filtering the salt from solution. In certain embodiments, Compound 1 is combined with about 1 to about 2 molar equivalents of L-(+)-tartaric acid. In certain embodiments, Compound 1 is combined with about 1 to about 1.5 molar equivalents of L-(+)-tartaric acid. In certain embodiments, Compound 1 is combined with about 1.2 molar equivalents of L-(+)-tartaric acid. In some embodiments, the solvent is a polar solvent. In some embodiments, the solvent is a C₁₋₆ alkyl alcohol. In some embodiments, the solvent is methanol. In some embodiments, the solvent is tetrahydrofuran. In some embodiments, the solvent is a halogenated solvent. In some embodiments, the solvent is a chlorinated solvent. In some embodiments, the solvent is dichloromethane. In some embodiments, the solvent is a mixture of one or more of tetrahydrofuran, methanol and dichloromethane.

Crystalline Form of Compound 1 L-(+)-tartrate

Provided herein are also processes for preparing a crystalline form of Compound 1 L-(+)-tartrate comprising recrystallizing Compound 1 L-(+)-tartrate in a recrystallizing solvent. In some embodiments, the recrystallizing comprises precipitating Compound 1 L-(+)-tartrate from a mixture of Compound 1 L-(+)-tartrate and the recrystallizing solvent. In some embodiments, the recrystallizing solvent is a polar solvent. In some embodiments, the recrystallizing solvent is a polar aprotic solvent. In some embodiments, the recrystallizing solvent is acetone. In some embodiments, the precipitating is performed at a reduced temperature. In some embodiments, the reduced temperature is from about 10° C. to about 30° C. In some embodiments, the precipitating is performed at room temperature.

In some embodiments, Compound 1 L-(+)-tartrate has at least one characteristic XRPD peak selected from about 11.7, about 13.9, about 15.2, about 21.8, and about 23.8 degrees 2-theta. In some embodiments, Compound 1 L-(+)-tartrate has at least two characteristic XRPD peaks selected from 11.7, about 13.9, about 15.2, about 21.8, and about 23.8 degrees 2-theta. In some embodiments, Compound 1 L-(+)-tartrate has at least three characteristic XRPD peaks selected from about 11.7, about 13.9, about 15.2, about 21.8, and about 23.8 degrees 2-theta. In some embodiments, Compound 1 L-(+)-tartrate has at least four characteristic XRPD peaks selected from about 11.7, about 13.9, about 15.2, about 21.8, and about 23.8 degrees 2-theta. In some embodiments, Compound 1 L-(+)-tartrate has at least five characteristic XRPD peaks selected from about 11.7, about 13.9, about 15.2, about 21.8, and about 23.8 degrees 2-theta. In some embodiments, Compound 1 L-(+)-tartrate has characteristic XRPD peaks at about 11.7, about 13.9, about 15.2, about 21.8, and about 23.8 degrees 2-theta. In some embodiments, Compound 1 L-(+)-tartrate has a characteristic XRPD peak at about 11.7 degrees 2-theta. In some embodiments, Compound 1 L-(+)-tartrate has a characteristic XRPD peak at about 13.9 degrees 2-theta. In some embodiments, Compound 1 L-(+)-tartrate has a characteristic XRPD peak at about 15.2 degrees 2-theta. In some embodiments, Compound 1 L-(+)-tartrate has a characteristic XRPD peak at about 21.8 degrees 2-theta. In some embodiments, Compound 1 L-(+)-tartrate has a characteristic XRPD peak at about 23.8 degrees 2-theta.

In some embodiments, Compound 1 L-(+)-tartrate has at least one characteristic XRPD peak selected from about 11.7, about 12.6, about 13.9, about 15.2, about 15.6, about 17.0, about 18.5, about 21.8, and about 23.8 degrees 2-theta. In some embodiments, Compound 1 L-(+)-tartrate has at least two characteristic XRPD peaks selected from about 11.7, about 12.6, about 13.9, about 15.2, about 15.6, about 17.0, about 18.5, about 21.8, and about 23.8 degrees 2-theta. In some embodiments, Compound 1 L-(+)-tartrate has at least three characteristic XRPD peaks selected from about 11.7, about 12.6, about 13.9, about 15.2, about 15.6, about 17.0, about 18.5, about 21.8, and about 23.8 degrees 2-theta. In some embodiments, Compound 1 L-(+)-tartrate has at least four characteristic XRPD peaks selected from about 11.7, about 12.6, about 13.9, about 15.2, about 15.6, about 17.0, about 18.5, about 21.8, and about 23.8 degrees 2-theta. In some embodiments, Compound 1 L-(+)-tartrate has at least five characteristic XRPD peaks selected from about 11.7, about 12.6, about 13.9, about 15.2, about 15.6, about 17.0, about 18.5, about 21.8, and about 23.8 degrees 2-theta. In some embodiments, the Compound 1 L-(+)-tartrate has characteristic XRPD peaks at about 11.7, about 12.6, about 13.9, about 15.2, about 15.6, about 17.0, about 18.5, about 21.8, and about 23.8 degrees 2-theta.

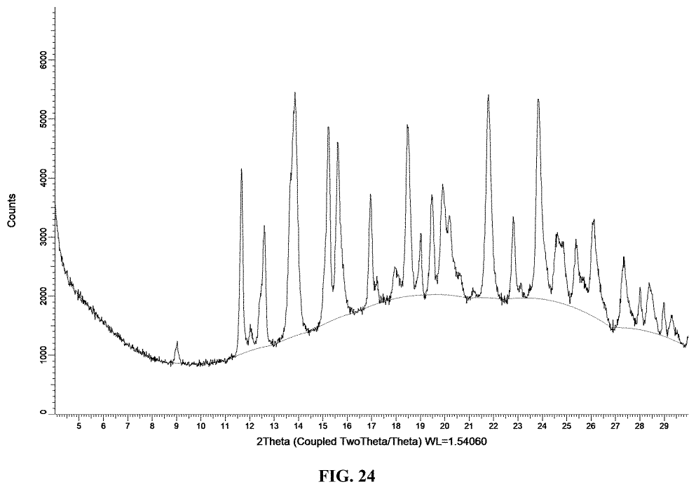

In some embodiments, Compound 1 L-(+)-tartrate has an XRPD pattern with characteristic peaks as substantially shown in FIG. 24.

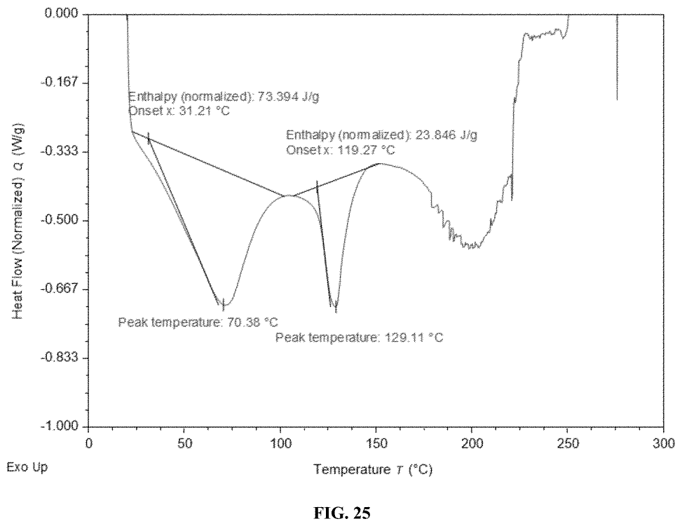

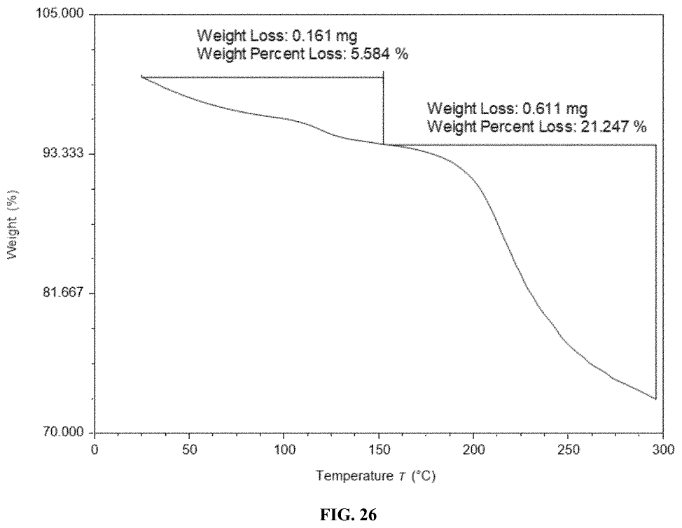

In some embodiments, Compound 1 L-(+)-tartrate exhibits a DSC thermogram having endotherm peaks at temperatures of about 70° C. and about 129° C. In some embodiments, Compound 1 L-(+)-tartrate exhibits a DSC thermogram having an endotherm peak at a temperature of about 70° C. In some embodiments, Compound 1 L-(+)-tartrate exhibits a DSC thermogram having an endotherm peak at a temperature of about 129° C. In some embodiments, Compound 1 L-(+)-tartrate has a DSC thermogram substantially as depicted in FIG. 25. In some embodiments, Compound 1 L-(+)-tartrate has a TGA thermogram substantially as depicted in FIG. 26.

In some embodiments, Compound 1 L-(+)-tartrate has at least one characteristic XRPD peak selected from about 11.7, about 13.9, about 15.2, about 21.8, and about 23.8 degrees 2-theta; and Compound 1 L-(+)-tartrate 1 exhibits a DSC thermogram having endotherm peaks at temperatures of about 70° C. and about 129° C. In some embodiments, Compound 1 L-(+)-tartrate has at least one characteristic XRPD peak selected from about 11.7, about 13.9, about 15.2, about 21.8, and about 23.8 degrees 2-theta; and Compound 1 L-(+)-tartrate exhibits a DSC thermogram having an endotherm peak at a temperatures of about 70° C. In some embodiments, Compound 1 L-(+)-tartrate has at least one characteristic XRPD peak selected from about 11.7, about 13.9, about 15.2, about 21.8, and about 23.8 degrees 2-theta; and Compound 1 L-(+)-tartrate exhibits a DSC thermogram having an endotherm peak at a temperatures of about 129° C.

In some embodiments, Compound 1 L-(+)-tartrate can be isolated with a purity of at least about 80%, about 85%, about 90%, about 95%, about 96%, about 97%, about 98%, or about 99%. In some embodiments, the Compound 1 L-(+)-tartrate can be isolated with a purity greater than about 99%.

### Malonic Acid Salts

Compound 1 malonate can be prepared by any suitable method for preparation of malonic acid addition salts. For example, Compound 1 can be combined with malonic acid (e.g., about 1.0 molar eq or more) in a solvent and the resulting salt can be isolated by filtering the salt from solution. In certain embodiments, Compound 1 is combined with about 1 to about 2 molar equivalents of malonic acid. In certain embodiments, Compound 1 is combined with about 1 to about 1.5 molar equivalents of malonic acid. In certain embodiments, Compound 1 is combined with about 1.2 molar equivalents of malonic acid. In some embodiments, the solvent is a polar solvent. In some embodiments, the solvent is a C₁₋₆ alkyl alcohol. In some embodiments, the solvent is methanol. In some embodiments, the solvent is tetrahydrofuran. In some embodiments, the solvent is a mixture of tetrahydrofuran and methanol.

Compound 1 malonate can be crystallized to provide a crystalline solid form. Provided herein are also processes for preparing Compound 1 malonate comprising recrystallizing Compound 1 malonate in a recrystallizing solvent. In some embodiments, the recrystallizing comprises precipitating Compound 1 malonate from a mixture of Compound 1 malonate and the recrystallizing solvent. In some embodiments, the recrystallizing solvent is a polar solvent. In some embodiments, the recrystallizing solvent is a polar aprotic solvent. In some embodiments, the recrystallizing solvent is acetone. In some embodiments, the precipitating is performed at a reduced temperature. In some embodiments, the reduced temperature is from about 10° C. to about 30° C. In some embodiments, the precipitating is performed at room temperature.

In some embodiments, Compound 1 malonate has at least one characteristic XRPD peak selected from about 4.0, about 9.0, about 13.9, about 17.1, about 17.9, about 18.8, and about 22.7 degrees 2-theta. In some embodiments, Compound 1 malonate has at least two characteristic XRPD peaks selected from 4.0, about 9.0, about 13.9, about 17.1, about 17.9, about 18.8, and about 22.7 degrees 2-theta. In some embodiments, Compound 1 malonate has at least three characteristic XRPD peaks selected from about 4.0, about 9.0, about 13.9, about 17.1, about 17.9, about 18.8, and about 22.7 degrees 2-theta. In some embodiments, Compound 1 malonate has at least four characteristic XRPD peaks selected from about 4.0, about 9.0, about 13.9, about 17.1, about 17.9, about 18.8, and about 22.7 degrees 2-theta. In some embodiments, Compound 1 malonate has at least five characteristic XRPD peaks selected from about 4.0, about 9.0, about 13.9, about 17.1, about 17.9, about 18.8, and about 22.7 degrees 2-theta. In some embodiments, Compound 1 malonate has characteristic XRPD peaks at about 4.0, about 9.0, about 13.9, about 17.1, about 17.9, about 18.8, and about 22.7 degrees 2-theta. In some embodiments, Compound 1 malonate has a characteristic XRPD peak at about 4.0 degrees 2-theta. In some embodiments, Compound 1 malonate has a characteristic XRPD peak at about 9.0 degrees 2-theta. In some embodiments, Compound 1 malonate has a characteristic XRPD peak at about 13.9 degrees 2-theta. In some embodiments, Compound 1 malonate has a characteristic XRPD peak at about 17.1 degrees 2-theta. In some embodiments, Compound 1 malonate has a characteristic XRPD peak at about 17.9 degrees 2-theta. In some embodiments, Compound 1 malonate has a characteristic XRPD peak at about 18.8 degrees 2-theta. In some embodiments, Compound 1 malonate has a characteristic XRPD peak at about 22.7 degrees 2-theta.

In some embodiments, Compound 1 malonate has at least one characteristic XRPD peak selected from about 4.0, about 9.0, about 13.1, about 13.9, about 14.0, about 17.1, about 17.9, about 18.8, about 20.7, and about 22.7 degrees 2-theta. In some embodiments, Compound 1 malonate has at least two characteristic XRPD peaks selected from about 4.0, about 9.0, about 13.1, about 13.9, about 14.0, about 17.1, about 17.9, about 18.8, about 20.7, and about 22.7 degrees 2-theta. In some embodiments, Compound 1 malonate has at least three characteristic XRPD peaks selected from about 4.0, about 9.0, about 13.1, about 13.9, about 14.0, about 17.1, about 17.9, about 18.8, about 20.7, and about 22.7 degrees 2-theta. In some embodiments, Compound 1 malonate has at least four characteristic XRPD peaks selected from about 4.0, about 9.0, about 13.1, about 13.9, about 14.0, about 17.1, about 17.9, about 18.8, about 20.7, and about 22.7 degrees 2-theta. In some embodiments, Compound 1 malonate has at least five characteristic XRPD peaks selected from about 4.0, about 9.0, about 13.1, about 13.9, about 14.0, about 17.1, about 17.9, about 18.8, about 20.7, and about 22.7 degrees 2-theta. In some embodiments, Compound 1 malonate has characteristic XRPD peaks at about 4.0, about 9.0, about 13.1, about 13.9, about 14.0, about 17.1, about 17.9, about 18.8, about 20.7, and about 22.7 degrees 2-theta.

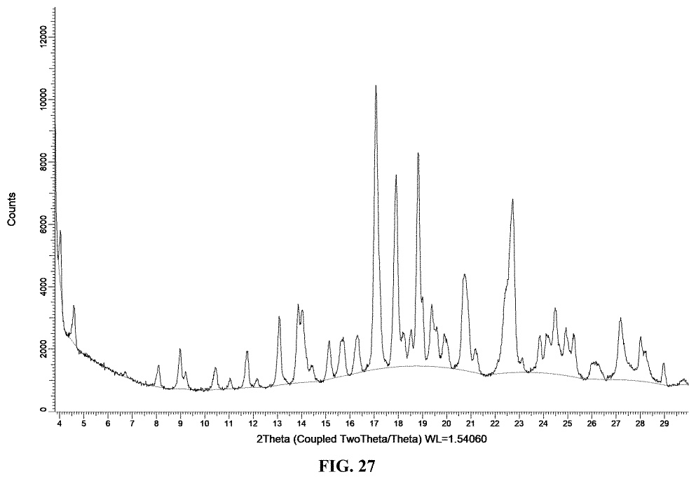

In some embodiments, Compound 1 malonate has an XRPD pattern with characteristic peaks as substantially shown in FIG. 27.

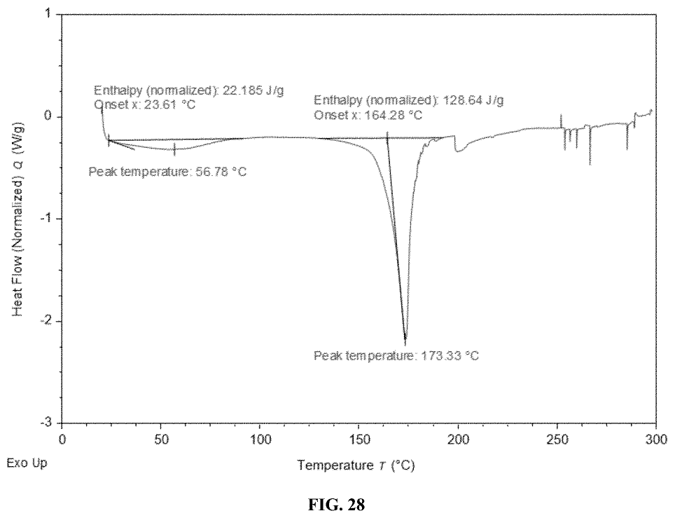

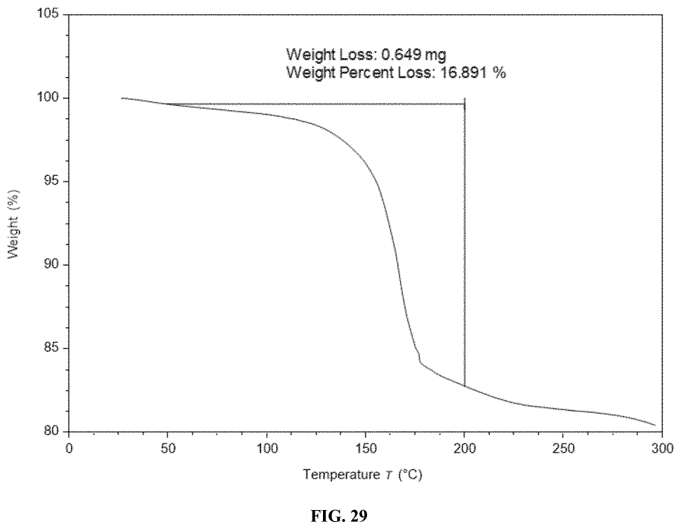

In some embodiments, Compound 1 malonate exhibits a DSC thermogram having endotherm peaks at temperatures of about 57° C. and about 173° C. In some embodiments, Compound 1 malonate exhibits a DSC thermogram having an endotherm peak at a temperature of about 57° C. In some embodiments, Compound 1 malonate exhibits a DSC thermogram having an endotherm peak at a temperature of about 173° C. In some embodiments, Compound 1 malonate has a DSC thermogram substantially as depicted in FIG. 28. In some embodiments, Compound 1 malonate has a TGA thermogram substantially as depicted in FIG. 29.

In some embodiments, Compound 1 malonate has at least one characteristic XRPD peak selected from about 4.0, about 9.0, about 13.9, about 17.1, about 17.9, about 18.8, and about 22.7 degrees 2-theta; and Compound 1 malonate exhibits a DSC thermogram having endotherm peaks at temperatures of about 57° C. and about 173° C. In some embodiments, Compound 1 malonate has at least one characteristic XRPD peak selected from about 4.0, about 9.0, about 13.9, about 17.1, about 17.9, about 18.8, and about 22.7 degrees 2-theta; and Compound 1 malonate exhibits a DSC thermogram having an endotherm peak at a temperatures of about 57° C. In some embodiments, Compound 1 malonate has at least one characteristic XRPD peak selected from about 4.0, about 9.0, about 13.9, about 17.1, about 17.9, about 18.8, and about 22.7 degrees 2-theta; and Compound 1 malonate exhibits a DSC thermogram having an endotherm peak at a temperatures of about 173° C.

In some embodiments, Compound 1 malonate can be isolated with a purity of at least about 80%, about 85%, about 90%, about 95%, about 96%, about 97%, about 98%, or about 99%. In some embodiments, Compound 1 malonate can be isolated with a purity greater than about 99%.

### Methanesulfonic Acid Salts

Compound 1 mesylate can be prepared by any suitable method for preparation of methanesulfonic acid addition salts. For example, Compound 1 can be combined with methanesulfonic acid (e.g., about 1.0 molar eq or more) in a solvent and the resulting salt can be isolated by filtering the salt from solution. In certain embodiments, Compound 1 is combined with about 1 to about 2 molar equivalents of methanesulfonic acid. In certain embodiments, Compound 1 is combined with about 1 to about 1.5 molar equivalents of methanesulfonic acid. In certain embodiments, Compound 1 is combined with about 1.2 molar equivalents of methanesulfonic acid. In some embodiments, the solvent is a polar solvent. In some embodiments, the solvent is a C₁₋₆ alkyl alcohol. In some embodiments, the solvent is methanol. In some embodiments, the solvent is tetrahydrofuran. In some embodiments, the solvent is a mixture of tetrahydrofuran and methanol.

Compound 1 mesylate can be crystallized to provide a crystalline solid form. Provided herein are also processes for preparing Compound 1 mesylate comprising recrystallizing Compound 1 mesylate in a recrystallizing solvent. In some embodiments, the recrystallizing comprises precipitating Compound 1 mesylate from a mixture of Compound 1 mesylate and the recrystallizing solvent. In some embodiments, the recrystallizing solvent is a polar solvent. In some embodiments, the recrystallizing solvent is a polar aprotic solvent. In some embodiments, the recrystallizing solvent is acetone. In some embodiments, the precipitating is performed at a reduced temperature. In some embodiments, the reduced temperature is from about 10° C. to about 30° C. In some embodiments, the reduced temperature is about room temperature.

In some embodiments, Compound 1 mesylate has at least one characteristic XRPD peak selected from about 4.9, about 5.7, about 8.0, about 9.9, and about 22.2 degrees 2-theta. In some embodiments, Compound 1 mesylate has at least two characteristic XRPD peaks selected from 4.9, about 5.7, about 8.0, about 9.9, and about 22.2 degrees 2-theta. In some embodiments, Compound 1 mesylate has at least three characteristic XRPD peaks selected from about 4.9, about 5.7, about 8.0, about 9.9, and about 22.2 degrees 2-theta. In some embodiments, Compound 1 mesylate has at least four characteristic XRPD peaks selected from about 4.9, about 5.7, about 8.0, about 9.9, and about 22.2 degrees 2-theta. In some embodiments, Compound 1 mesylate has at least five characteristic XRPD peaks selected from about 4.9, about 5.7, about 8.0, about 9.9, and about 22.2 degrees 2-theta. In some embodiments, Compound 1 mesylate has characteristic XRPD peaks at about 4.9, about 5.7, about 8.0, about 9.9, and about 22.2 degrees 2-theta. In some embodiments, Compound 1 mesylate has a characteristic XRPD peak at about 4.9 degrees 2-theta. In some embodiments, Compound 1 mesylate has a characteristic XRPD peak at about 5.7 degrees 2-theta. In some embodiments, Compound 1 mesylate has a characteristic XRPD peak at about 8.0 degrees 2-theta. In some embodiments, Compound 1 mesylate has a characteristic XRPD peak at about 9.9 degrees 2-theta. In some embodiments, Compound 1 mesylate has a characteristic XRPD peak at about 22.2 degrees 2-theta.

In some embodiments, Compound 1 mesylate has at least one characteristic XRPD peak selected from about 4.9, about 5.7, about 8.0, about 9.9, about 11.8, about 19.6, about 20.0, about 20.6, and about 22.2 degrees 2-theta. In some embodiments, Compound 1 mesylate has at least two characteristic XRPD peaks selected from about 4.9, about 5.7, about 8.0, about 9.9, about 11.8, about 19.6, about 20.0, about 20.6, and about 22.2 degrees 2-theta. In some embodiments, Compound 1 mesylate has at least three characteristic XRPD peaks selected from about 4.9, about 5.7, about 8.0, about 9.9, about 11.8, about 19.6, about 20.0, about 20.6, and about 22.2 degrees 2-theta. In some embodiments, Compound 1 mesylate has at least four characteristic XRPD peaks selected from about 4.9, about 5.7, about 8.0, about 9.9, about 11.8, about 19.6, about 20.0, about 20.6, and about 22.2 degrees 2-theta. In some embodiments, Compound 1 mesylate has at least five characteristic XRPD peaks selected from about 4.9, about 5.7, about 8.0, about 9.9, about 11.8, about 19.6, about 20.0, about 20.6, and about 22.2 degrees 2-theta. In some embodiments, Compound 1 mesylate has characteristic XRPD peaks at about 4.9, about 5.7, about 8.0, about 9.9, about 11.8, about 19.6, about 20.0, about 20.6, and about 22.2 degrees 2-theta.

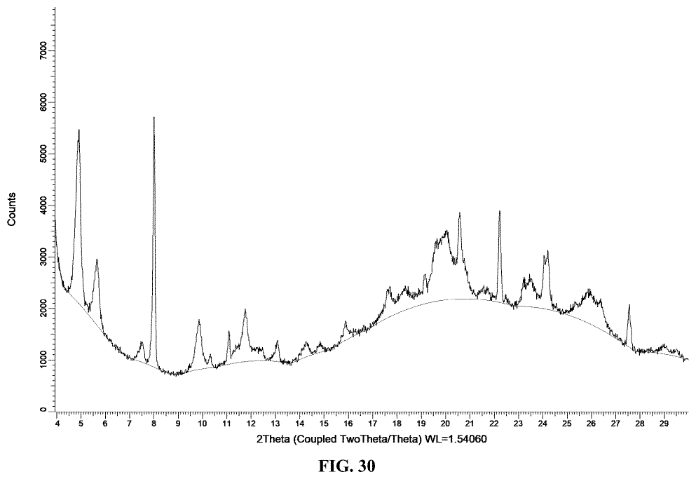

In some embodiments, Compound 1 mesylate has an XRPD pattern with characteristic peaks as substantially shown in FIG. 30.

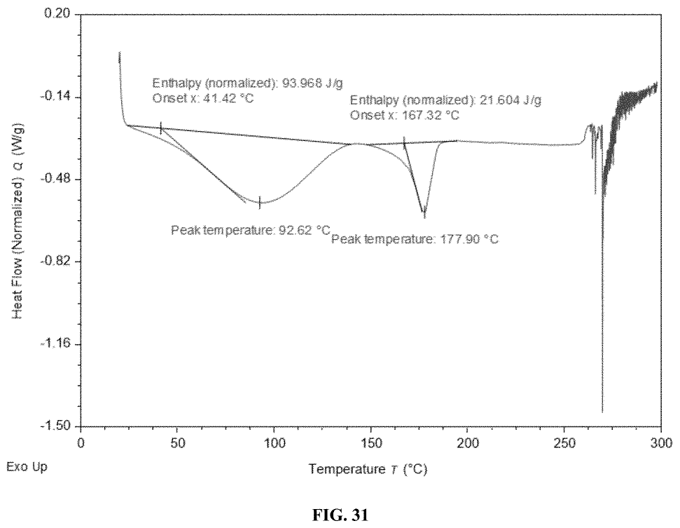

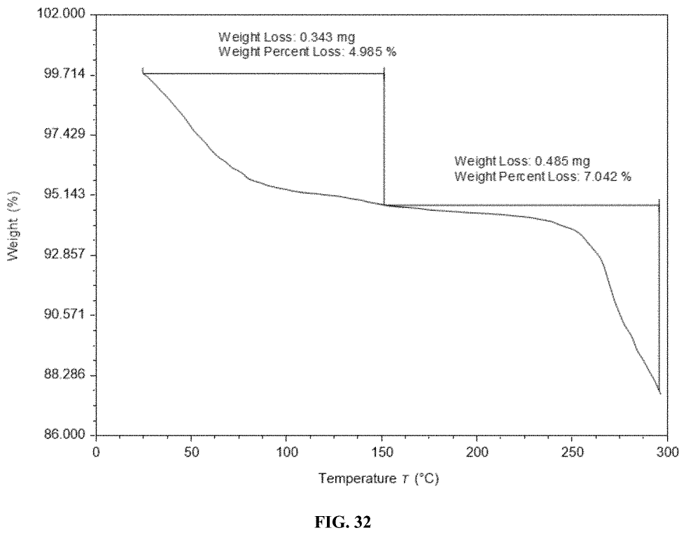

In some embodiments, Compound 1 mesylate exhibits a DSC thermogram having endotherm peaks at temperatures of about 93° C. and about 178° C. In some embodiments, Compound 1 mesylate exhibits a DSC thermogram having an endotherm peak at a temperature of about 93° C. In some embodiments, Compound 1 mesylate exhibits a DSC thermogram having an endotherm peak at a temperature of about 178° C. In some embodiments, Compound 1 mesylate has a DSC thermogram substantially as depicted in FIG. 31. In some embodiments, Compound 1 mesylate has a TGA thermogram substantially as depicted in FIG. 32.

In some embodiments, Compound 1 mesylate has at least one characteristic XRPD peak selected from about 4.9, about 5.7, about 8.0, about 9.9, and about 22.2 degrees 2-theta; and Compound 1 mesylate exhibits a DSC thermogram having endotherm peaks at temperatures of about 93° C. and about 178° C. In some embodiments, Compound 1 mesylate has at least one characteristic XRPD peak selected from about 4.9, about 5.7, about 8.0, about 9.9, and about 22.2 degrees 2-theta; and Compound 1 mesylate exhibits a DSC thermogram having an endotherm peak at a temperatures of about 93° C. In some embodiments, Compound 1 mesylate has at least one characteristic XRPD peak selected from about 4.9, about 5.7, about 8.0, about 9.9, and about 22.2 degrees 2-theta; and Compound 1 mesylate exhibits a DSC thermogram having an endotherm peak at a temperatures of about 178° C.

In some embodiments, Compound 1 mesylate can be isolated with a purity of at least about 80%, about 85%, about 90%, about 95%, about 96%, about 97%, about 98%, or about 99%. In some embodiments, Compound 1 mesylate can be isolated with a purity greater than about 99%.

### Adipic Acid Salts

Compound 1 adipate can be prepared by any suitable method for preparation of adipic acid addition salts. For example, Compound 1 can be combined with adipic acid (e.g., about 1.0 molar eq or more) in a solvent and the resulting salt can be isolated by filtering the salt from solution. In certain embodiments, Compound 1 is combined with about 1 to about 2 molar equivalents of adipic acid. In certain embodiments, Compound 1 is combined with about 1 to about 1.5 molar equivalents of adipic acid. In certain embodiments, Compound 1 is combined with about 1.2 molar equivalents of adipic acid. In some embodiments, the solvent is a polar solvent. In some embodiments, the solvent is a C₁₋₆ alkyl alcohol. In some embodiments, the solvent is methanol.

### Fumaric Acid Salts

Compound 1 fumarate can be prepared by any suitable method for preparation of fumaric acid addition salts. For example, Compound 1 can be combined with fumaric acid (e.g., about 1.0 molar eq or more) in a solvent and the resulting salt can be isolated by filtering the salt from solution. In certain embodiments, Compound 1 is combined with about 1 to about 2 molar equivalents of fumaric acid. In certain embodiments, Compound 1 is combined with about 1 to about 1.5 molar equivalents of fumaric acid. In certain embodiments, Compound 1 is combined with about 1.2 molar equivalents of fumaric acid. In some embodiments, the solvent is a polar solvent. In some embodiments, the solvent is a C₁₋₆ alkyl alcohol. In some embodiments, the solvent is methanol.

### Maleic Acid Salts

Compound 1 maleate can be prepared by any suitable method for preparation of maleic acid addition salts. For example, Compound 1 can be combined with maleic acid (e.g., about 1.0 molar eq or more) in a solvent and the resulting salt can be isolated by filtering the salt from solution. In certain embodiments, Compound 1 is combined with about 1 to about 2 molar equivalents of maleic acid. In certain embodiments, Compound 1 is combined with about 1 to about 1.5 molar equivalents of maleic acid. In certain embodiments, Compound 1 is combined with about 1.2 molar equivalents of maleic acid. In some embodiments, the solvent is a polar solvent. In some embodiments, the solvent is a C₁₋₆ alkyl alcohol. In some embodiments, the solvent is methanol.

### Malic Acid Salts

Compound 1 malate can be prepared by any suitable method for preparation of malic acid addition salts. For example, Compound 1 can be combined with L-(−)-malic acid (e.g., about 1.0 molar eq or more) in a solvent and the resulting salt can be isolated by filtering the salt from solution. In certain embodiments, Compound 1 is combined with about 1 to about 2 molar equivalents of L-(−)-malic acid. In certain embodiments, Compound 1 is combined with about 1 to about 1.5 molar equivalents of L-(−)-malic acid. In certain embodiments, Compound 1 is combined with about 1.2 molar equivalents of L-(−)-malic acid. In some embodiments, the solvent is a polar solvent. In some embodiments, the solvent is a C₁₋₆ alkyl alcohol. In some embodiments, the solvent is methanol.

### Succinic Acid Salts

Compound 1 succinate can be prepared by any suitable method for preparation of succinic acid addition salts. For example, Compound 1 can be combined with succinic acid (e.g., about 1.0 molar eq or more) in a solvent and the resulting salt can be isolated by filtering the salt from solution. In certain embodiments, Compound 1 is combined with about 1 to about 2 molar equivalents of succinic acid. In certain embodiments, Compound 1 is combined with about 1 to about 1.5 molar equivalents of succinic acid. In certain embodiments, Compound 1 is combined with about 1.2 molar equivalents of succinic acid. In some embodiments, the solvent is a polar solvent. In some embodiments, the solvent is a C₁₋₆ alkyl alcohol. In some embodiments, the solvent is methanol.

### Processes for Preparing Compound 1 or a Salt Thereof

Provided herein are also processes for preparing Compound 1 or a salt thereof. The processes for preparing Compound 1 or a salt thereof provided herein have certain advantages over the processes currently disclosed in the art. For example, the processes described herein demonstrate good scalability and yields.

The present application further provides a process of preparing Compound 1, where the process can be suitable for scale up. A process of preparing Compound 1 is described in US 2021/0106588, the entirety of which is incorporated herein by reference. In comparison to the process described in US 2021/0106588, the process provided herein has certain advantages making it suitable for scale up. For example, the process provided herein affords high yields, good quality products, and do not require purification by column chromatography. Additionally, the amine-protecting groups (e.g., the Boc protecting group) used in the synthesis of Compound 1 disclosed in US 2021/0106588 was labile to the disclosed reaction conditions, as deprotected amine-compounds could be observed and isolated. In contrast, the t-butyl protecting group of the present synthesis was stable to the various reaction conditions.

Provided herein is a process for preparing Compound 1:

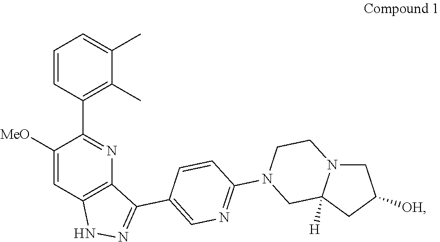 [SMILES: COc1cc2[nH]nc(-c3ccc(N4CCN5C[C@H](O)C[C@H]5C4)nc3)c2nc1-c1cccc(C)c1C]

or a salt thereof, comprising:

(i) halogenating Compound 7 having the formula:

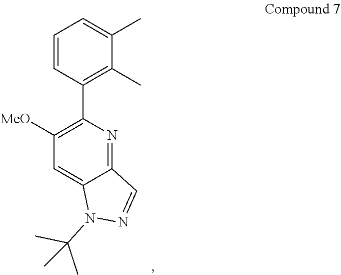 [SMILES: COc1cc2c(cnn2C(C)(C)C)nc1-c1cccc(C)c1C]

or a salt thereof, with H1, wherein H1 is a halogenating agent, to provide Compound 5:

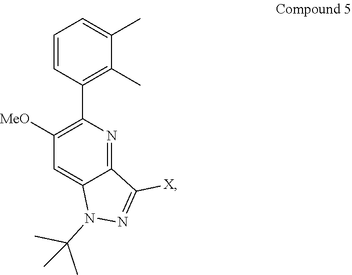

or a salt thereof, wherein X is halo;

(ii) coupling Compound 5, or a salt thereof, with Compound 6 having the formula:

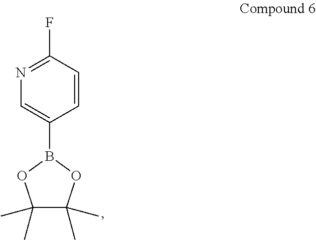

or a salt thereof, in the presence of CA1 and B2, wherein CA1 is a catalyst and B2 is a base, to provide Compound 3:

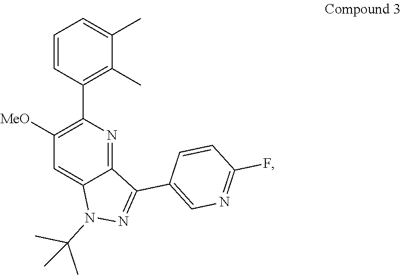 [SMILES: COc1cc2c(nc1-c1cccc(C)c1C)c(-c1ccc(F)nc1)nn2C(C)(C)C]

or a salt thereof;

(iii) contacting Compound 3, or a salt thereof, with Compound 4 having the formula:

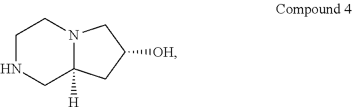

or a salt thereof, in the presence of B1, wherein B1 is a base, to provide Compound 2:

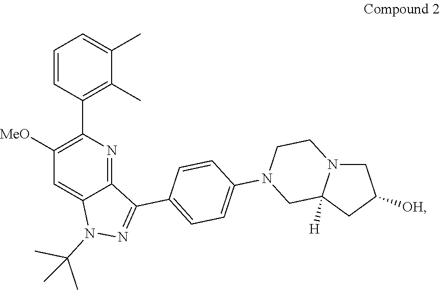 [SMILES: COc1cc2c(nc1-c1cccc(C)c1C)c(-c1ccc(N3CCN4C[C@H](O)C[C@H]4C3)cc1)nn2C(C)(C)C]

or a salt thereof; and

(iv) deprotecting Compound 2, or a salt thereof, with A1, wherein A1 is an acid, to provide Compound 1, or a salt thereof.

Compound 7, or a salt thereof, can be prepared by a process comprising:

(i) treating Compound 10 having the formula:

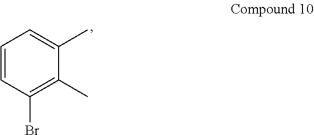

or a salt thereof, with magnesium in the presence of 2-methoxyacetonitrile, to provide Compound 8 having the formula:

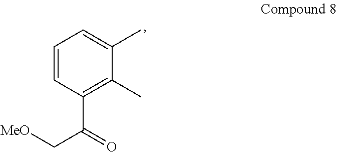

or a salt thereof;

(ii) contacting Compound 8, or a salt thereof, with Compound 9 having the formula:

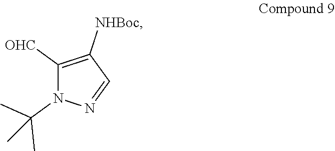 [SMILES: CC(C)(C)OC(=O)Nc1cnn(C(C)(C)C)c1C=O]

or a salt thereof, in the presence of A2, wherein A2 is an acid, to provide Compound 7, or a salt thereof.

Compound 9, or a salt thereof, can be prepared by a process comprising:

(i) treating Compound 13 having the formula:

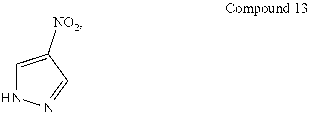

or a salt thereof, with tert-butyl acetate in the presence of A3, wherein A3 is an acid, to provide Compound 12 having the formula:

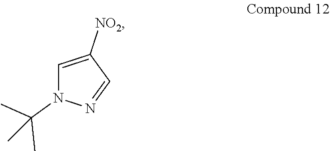

or a salt thereof;

(ii) reducing Compound 12, or a salt thereof, in the presence of di-tert-butyl dicarbonate and CA2, wherein CA2 is a catalyst, to provide Compound 11 having the formula:

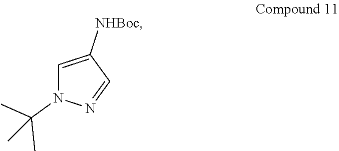 [SMILES: CC(C)(C)OC(=O)Nc1cnn(C(C)(C)C)c1]

or a salt thereof;

(iii) reacting Compound 11 having the formula:

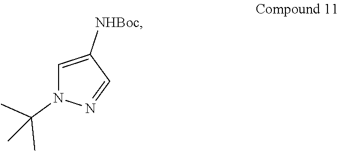 [SMILES: CC(C)(C)OC(=O)Nc1cnn(C(C)(C)C)c1]

or a salt thereof, with B3 followed by N,N-dimethylformamide, wherein B3 is a base, to provide Compound 9, or a salt thereof.

Provided herein is a process for preparing Compound 1, or a salt thereof, comprising deprotecting Compound 2, or a salt thereof, with A1, wherein A1 is an acid. In some embodiments, A1 is an inorganic acid. In some embodiments, A1 is sulfuric acid. In some embodiments, the deprotecting is performed in the presence of S1, wherein S1 is a protic solvent. In some embodiments, S1 is water. In some embodiments, the deprotecting comprises using about 1 to about 20 molar equivalents of A1 relative to Compound 2. In some embodiments, the deprotecting comprises using about 10 to about 15 molar equivalents of A1 relative to Compound 2. In some embodiments, the deprotecting comprises using about 12 molar equivalents of A1 relative to Compound 2. In some embodiments, the deprotecting is performed at a temperature from about 90° C. to about 120° C.

Compound 2, or a salt thereof, can be prepared by a process comprising contacting Compound 3, or a salt thereof, with Compound 4, or a salt thereof, in the presence of B1, wherein B1 is a base. In some embodiments, about 1 to about 5 molar equivalent of Compound 4 is present relative to Compound 3. In some embodiments, about 1 to about 2 molar equivalent of Compound 4 is present relative to Compound 3. In some embodiments, about 1.1 molar equivalents of Compound 4 is present relative to Compound 3. In some embodiments, B1 is an organolithium base. In some embodiments, B1 is n-butyllithium. In some embodiments, the process further comprises treating Compound 4 with B1 before the contacting of Compound 3 with Compound 4. In some embodiments, the contacting is performed in the presence of S2, wherein S2 is a polar aprotic solvent. In some embodiments, S2 is tetrahydrofuran. In some embodiments, the contacting comprises using about 1 to about 10 molar equivalents of B1 relative to Compound 3. In some embodiments, the contacting comprises using about 1 to about 5 molar equivalents of B1 relative to Compound 3. In some embodiments, contacting comprises using about 4.0 molar equivalent of B1 relative to Compound 3. In some embodiments, the contacting is performed at a temperature from about 10° C. to about 40° C. In some embodiments, the contacting is performed at room temperature.

Compound 3, or a salt thereof, can be prepared by a process comprising coupling Compound 5, or a salt thereof, with Compound 6, or a salt thereof, in the presence of CA1 and B2, wherein CA1 is a catalyst and B2 is a base. In some embodiments, the coupling of Compounds 5 and 6 comprises about 1 to about 5 molar equivalents of Compound 6 relative to Compound 5. In some embodiments, the coupling of Compounds 5 and 6 comprises using about 1 to about 2 molar equivalents of Compound 6 relative to Compound 5. In some embodiments, the coupling of Compounds 5 and 6 comprises using about 1.1 molar equivalents of Compound 6 relative to Compound 5. In some embodiments, CA1 is a palladium catalyst. In some embodiments, CA1 is bis(di-tert-butyl(4-dimethylaminophenyl)phosphine)dichloropalladium (II) (Pd-132). In some embodiments, about 0.0001 to about 0.01 molar equivalents of CA1 is present relative to compound 5. In some embodiments, about 0.001 molar equivalents of CA1 is present relative to compound 5. In some embodiments, B2 is an inorganic base. In some embodiments, B2 is a phosphate base. In some embodiments, B2 is potassium phosphate tribasic monohydrate. In some embodiments, the coupling is performed in the presence of S3, wherein S3 is a mixture of a polar aprotic solvent and a protic solvent. In some embodiments, S3 is a mixture of tetrahydrofuran and water. In some embodiments, the coupling of Compounds 5 and 6 comprises using about 1 to about 10 molar equivalents of B2 relative to Compound 5. In some embodiments, the coupling of Compounds 5 and 6 comprises using about 1 to about 5 molar equivalents of B2 relative to Compound 5. In some embodiments, the coupling of Compounds 5 and 6 comprises using about 2.5 molar equivalents of B2 relative to Compound 5. In some embodiments, the coupling of Compounds 5 and 6 is performed at a temperature from about 20° C. to about 100° C. In some embodiments, the coupling of Compounds 5 and 6 is performed at a temperature of about 56° C. to about 76° C.

In some embodiments, X is Br. In some embodiments, Compound 5 has the structure of Compound 5a:

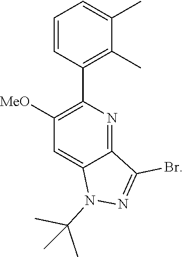 [SMILES: COc1cc2c(nc1-c1cccc(C)c1C)c(Br)nn2C(C)(C)C]

Compound 5, or a salt thereof, can be prepared by a process comprising halogenating Compound 7, or a salt thereof, with H1, wherein H1 is a halogenating agent. In some embodiments, H1 is a brominating reagent. In some embodiments, H1 is N-bromosuccinimide. In some embodiments, the halogenating is performed in the presence of S4, wherein S4 is a polar aprotic solvent. In some embodiments, S4 is acetonitrile. In some embodiments, the halogenating comprises using about 1 to about 10 molar equivalents of H1 relative to Compound 7. In some embodiments, the halogenating comprises using about 1 to about 2 molar equivalents of H1 relative to Compound 7. In some embodiments, the halogenating comprises using about 1 molar equivalent of H1 relative to Compound 7. In some embodiments, the halogenating is performed at a temperature from about 40° C. to about 70° C. In some embodiments, the halogenating is performed at a temperature of about 55° C.

Compound 7, or a salt thereof, can be prepared by a process comprising contacting Compound 8, or a salt thereof, with Compound 9, or a salt thereof, in the presence of A2, wherein A2 is an acid. In some embodiments, the contacting of Compound 8 and Compound 9 comprises about 0.1 to about 10 molar equivalent of Compound 9 relative to Compound 8. In some embodiments, the contacting of Compound 8 and Compound 9 comprises using about 0.2 to about 5 molar equivalents of Compound 9 relative to Compound 8. In some embodiments, the contacting of Compound 8 and Compound 9 comprises using about 1 molar equivalent of Compound 9 relative to Compound 8. In some embodiments, A2 is a sulfonic acid. In some embodiments, A2 is methanesulfonic acid. In some embodiments, the contacting of Compound 8 and Compound 9 is performed in the presence of S5, wherein S5 is a protic solvent. In some embodiments, S5 is ethanol. In some embodiments, the contacting of Compound 8 and Compound 9 comprises using about 1 to about 10 molar equivalents of A2 relative to Compound 8. In some embodiments, the contacting of Compound 8 and Compound 9 comprises using about 1 to about 2 molar equivalents of A2 relative to Compound 8. In some embodiments, the contacting of Compound 8 and Compound 9 comprises using about 1.2 molar equivalents of A2 relative to Compound 8. In some embodiments, the contacting of Compound 8 and Compound 9 is performed at a temperature from about 55° C. to about 75° C. In some embodiments, the contacting of Compound 8 and Compound 9 is performed at a temperature of about 65° C.

Compound 8, or a salt thereof, can be prepared by a process comprising treating Compound 10, or a salt thereof, with magnesium in the presence of 2-methoxyacetonitrile. In some embodiments, the treating of Compound 10 with magnesium is performed in presence of iodine. In some embodiments, the treating of Compound 10 with magnesium is performed in the presence of S6, wherein S6 is a polar aprotic solvent. In some embodiments, S6 is tetrahydrofuran. In some embodiments, the treating of Compound 10 with magnesium comprises using about 1 to about 10 molar equivalents of magnesium relative to Compound 10. In some embodiments, the treating of Compound 10 with magnesium comprises using about 1 to about 5 molar equivalents of magnesium relative to Compound 10. In some embodiments, the treating of Compound 10 with magnesium comprises using about 1.1 molar equivalents of magnesium relative to Compound 10. In some embodiments, the treating of Compound 10 with magnesium comprises using about 0.0001 to about 0.01 molar equivalents of iodine relative to Compound 10. In some embodiments, the treating of Compound 10 with magnesium comprises using about 0.0012 molar equivalents of iodine relative to Compound 10. In some embodiments, the treating of Compound 10 and 2-methoxyacetonitrile is further performed in the presence of S7, wherein S7 is a polar aprotic solvent. In some embodiments, S7 is tetrahydrofuran. In some embodiments, the treating of Compound 10 with magnesium is performed at a temperature from about 55° C. to about 75° C. In some embodiments, the treating of Compound 10 with magnesium is performed at a temperature of about 65° C. In some embodiments, the treating of Compound 10 and 2-methoxyacetonitrile comprises using about 1 to about 10 molar equivalents of 2-methoxyacetonitrile relative to Compound 10. In some embodiments, the treating of Compound 10 and 2-methoxyacetonitrile comprises using about 1 to about 5 molar equivalents of 2-methoxyacetonitrile relative to Compound 10. In some embodiments, the treating of Compound 10 and 2-methoxyacetonitrile comprises using about 1 molar equivalent of 2-methoxyacetonitrile relative to Compound 10. In some embodiments, the treating of Compound 10 and 2-methoxyacetonitrile is performed at a temperature from about 20° C. to about 30° C.

Compound 9, or a salt thereof, can be prepared by a process comprising reacting Compound 11, or a salt thereof, with B3 then N,N-dimethylformamide, wherein B3 is a base. In some embodiments, B3 is an organolithium base. In some embodiments, B3 is n-butyllithium. In some embodiments, the reacting of Compound 11 with B3 and N,N-dimethylformamide is performed in the presence of S8, wherein S8 is a polar aprotic solvent. In some embodiments, S8 is tetrahydrofuran. In some embodiments, the reacting of Compound 11 with B3 comprises using about 1 to about 10 molar equivalents of B3 relative to Compound 11. In some embodiments, the reacting of Compound 11 with B3 comprises using about 1 to about 5 molar equivalents of B3 relative to Compound 11. In some embodiments, the reacting of Compound 11 with B3 comprises using about 2.2 molar equivalents of B3 relative to Compound 11. In some embodiments, the reacting of Compound 11 with N,N-dimethylformamide comprises using about 1 to about 10 molar equivalents of N,N-dimethylformamide relative to Compound 11. In some embodiments, the reacting of Compound 11 with N,N-dimethylformamide comprises using about 1 to about 5 molar equivalents of N,N-dimethylformamide relative to Compound 11. In some embodiments, the reacting of Compound 11 with N,N-dimethylformamide comprises using about 1.3 molar equivalents of N,N-dimethylformamide relative to Compound 11. In some embodiments, the reacting of Compound 11 with B3 is performed at a temperature from about 65° C. to about 85° C. In some embodiments, the reacting of Compound 11 with B3 is performed at a temperature of about 76° C. In some embodiments, the reacting of Compound 11 with N,N-dimethylformamide is performed at a temperature from about 65° C. to about 85° C. In some embodiments, the reacting of Compound 11 with N,N-dimethylformamide is performed at a temperature of about 76° C.

Compound 11, or a salt thereof, can be prepared by a process comprising reducing Compound 12, or a salt thereof, in the presence of di-tert-butyl dicarbonate and CA2, wherein CA2 is a catalyst. In some embodiments, the reducing of Compound 12 is performed under a hydrogen atmosphere. In some embodiments, the reducing of Compound 12 is performed under about 30 to about 50 psi of hydrogen gas. In some embodiments, the reducing of Compound 12 is performed under about 40 psi of hydrogen gas. In some embodiments, CA2 is a hydrogenation catalyst. In some embodiments, CA2 is 10% palladium on carbon. In some embodiments, the reducing of Compound 12 is performed in the presence of S9, wherein S9 is a protic solvent. In some embodiments, S9 is methanol. In some embodiments, the reducing of Compound 12 in the presence of di-tert-butyl dicarbonate comprises using about 1 to about 10 molar equivalents of di-tert-butyl dicarbonate relative to the Compound 12. In some embodiments, the reducing of Compound 12 in the presence of di-tert-butyl dicarbonate comprises using about 1 to about 5 molar equivalents of di-tert-butyl dicarbonate relative to the Compound 12. In some embodiments, the reducing of Compound 12 in the presence of di-tert-butyl dicarbonate comprises using about 1.1 molar equivalents of di-tert-butyl dicarbonate relative to the Compound 12. In some embodiments, the reducing of Compound 12 in the presence of di-tert-butyl dicarbonate is performed at a temperature from about 10° C. to about 40° C. In some embodiments, the reducing of Compound 12 in the presence of di-tert-butyl dicarbonate is performed at room temperature.

Compound 12, or a salt thereof, can be prepared by a process comprising treating Compound 13, or a salt thereof, with tert-butyl acetate in the presence of A3, wherein A3 is an acid. In some embodiments, A3 is an inorganic acid. In some embodiments, A3 is sulfuric acid. In some embodiments, the treating of Compound 13 is performed in the presence of S10, wherein S10 is a polar aprotic solvent. In some embodiments, S10 is 1,4-dioxane. In some embodiments, the treating of Compound 13 comprises using about 1 to about 10 molar equivalents of tert-butyl acetate relative to Compound 13. In some embodiments, the treating of Compound 13 comprises using about 1 to about 5 molar equivalents of tert-butyl acetate relative to Compound 13. In some embodiments, the treating of Compound 13 comprises using about 3 molar equivalents of tert-butyl acetate relative to Compound 13. In some embodiments, the treating of Compound 13 comprises using about 1 to about 10 molar equivalents of A3 relative to Compound 13. In some embodiments, the treating of Compound 13 comprises using about 1 to about 5 molar equivalents of A3 relative to Compound 13. In some embodiments, the treating of Compound 13 comprises using about 1.4 molar equivalents of A3 relative to Compound 12. In some embodiments, the treating of Compound 13 is performed at a temperature from about 50° C. to about 70° C. In some embodiments, the treating of Compound 13 is performed at a temperature of about 60° C.

### Intermediates

Provided herein is a compound having the formula:

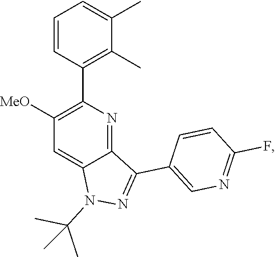 [SMILES: COc1cc2c(nc1-c1cccc(C)c1C)c(-c1ccc(F)nc1)nn2C(C)(C)C]

or a salt thereof.

Provided herein is a compound having the formula:

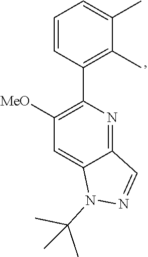 [SMILES: COc1cc2c(cnn2C(C)(C)C)nc1-c1cccc(C)c1C]

or a salt thereof.

Provided herein is a compound having the formula:

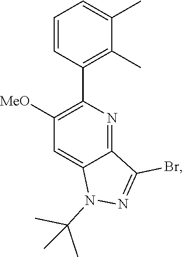 [SMILES: COc1cc2c(nc1-c1cccc(C)c1C)c(Br)nn2C(C)(C)C]

or a salt thereof.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

As used herein, the term “reacting,” “contacting” or “treating” when describing a certain process is used as known in the art and generally refers to the bringing together of chemical reagents in such a manner so as to allow their interaction at the molecular level to achieve a chemical or physical transformation. In some embodiments, the reacting involves two reagents, wherein one or more equivalents of second reagent are used with respect to the first reagent. The reacting steps of the processes described herein can be conducted for a time and under conditions suitable for preparing the identified product.

The terms “protecting” and “deprotecting” as used herein in a chemical reaction refer to inclusion of a chemical group in a process and such group is removed in a later step in the process. The term preparation of Compound 1 and its salts can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006). Examples of protecting groups include amino protecting groups. As used herein, “amino protecting group” refers to any protecting group for the protection of amines. Example amino protecting groups include, but are not limited to, phenylsulfonyl, benzyloxycarbonyl (Cbz), 2,2,2-trichloroethoxycarbonyl (Troc), 2-(trimethylsilyl)ethoxycarbonyl (Teoc), 2-(4-trifluoromethylphenylsulfonyl)ethoxycarbonyl (Tsc), t-butoxycarbonyl (BOC), 1-adamantyloxycarbonyl (Adoc), 2-adamantylcarbonyl (2-Adoc), 2,4-dimethylpent-3-yloxycarbonyl (Doc), cyclohexyloxycarbonyl (Hoc), 1,1-dimethyl-2,2,2-trichloroethoxycarbonyl (TcBOC), vinyl, 2-chloroethyl, 2-phenylsulfonylethyl, allyl, benzyl, 2-nitrobenzyl, 4-nitrobenzyl, diphenyl-4-pyridylmethyl, N′,N′-dimethylhydrazinyl, methoxymethyl, t-butoxymethyl (Bum), benzyloxymethyl (BOM), or 2-tetrahydropyranyl (THP), tri(C₁₋₄alkyl)silyl (e.g., tri(isopropyl)silyl), 1,1-diethoxymethyl, or N-pivaloyloxymethyl (POM). A further example of an amino protecting group is a tert-butyl group.

The term “inorganic base” refers to a base acid that is formed from an inorganic compound and can accept hydrogen ions to form conjugate acid ions in an aqueous solution. An inorganic base can be a strong base or a weak base. Examples of inorganic bases acids include, but are not limited to potassium perchlorate, potassium phosphate tribasic monohydrate, and the like.

The term “organolithium base” refers to organometallic compounds that contain carbon-lithium bonds. Examples of an organolithium base include tert-butyllithium, n-butyllithium, sec-butyllithium, and the like.

The term “phosphate base” refers to a phosphate anion that is basic. Examples of an phosphate base include potassium phosphate tribasic monohydrate, potassium phosphate sibasic monohydrate, and the like.

The term “inorganic acid” refers to an acid that is formed from an inorganic compound and can form hydrogen ions and conjugate base ions in an aqueous solution. Inorganic acids can be a strong acid or weak acid. Examples of inorganic acids include but not limited to hydrochloric acid, perchloric acid, sulfuric acid, nitric acid, boric acid, phosphoric acid, and the like.

The term “sulfonic acid” refers to an acid with a sulfo group, or a group having the general formula R—S(O)₂—OH, wherein R is an alkyl or aryl group. Examples of sulfonic acids include, sulfuric acid, methanesulfonic acid, p-toluenesulfonic acid, and the like.

In some embodiments, anti-solvent as described herein refers to a solvent where Compound 1 or its salts are less soluble relative to another solvent or solvent mixture in the solution. For example, anti-solvent can include but not limited to benzene, cyclohexane, pentane, hexane, heptane (e.g., n-heptane), toluene, cycloheptane, methylcyclohexane, heptane, ethylbenzene, m-, o-, or p-xylene, octane, indane, nonane, or naphthalene.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected. In some embodiments, reactions can be carried out in the absence of solvent, such as when at least one of the reagents is a liquid or gas.

Suitable solvents can include halogenated solvents such as carbon tetrachloride, bromodichloromethane, dibromochloromethane, bromoform, chloroform, bromochloromethane, dibromomethane, butyl chloride, dichloromethane (methylene chloride), tetrachloroethylene, trichloroethylene, 1,1,1-trichloroethane, 1,1,2-trichloroethane, 1,1-dichloroethane, 2-chloropropane, α,α,α-trifluorotoluene, 1,2-dichloroethane, 1,2-dibromoethane, hexafluorobenzene, 1,2,4-trichlorobenzene, 1,2-dichlorobenzene, chlorobenzene, fluorobenzene, mixtures thereof and the like.

Suitable ether solvents include: dimethoxymethane, tetrahydrofuran, 1,3-dioxane, 1,4-dioxane, furan, tetrahydrofuran (THF), diethyl ether, ethylene glycol dimethyl ether, ethylene glycol diethyl ether, diethylene glycol dimethyl ether (diglyme), diethylene glycol diethyl ether, triethylene glycol dimethyl ether, anisole, tert-butyl methyl ether, mixtures thereof and the like.

Suitable polar protic solvents can include, by way of example and without limitation, water, methanol, ethanol, 2-nitroethanol, 2-fluoroethanol, 2,2,2-trifluoroethanol, ethylene glycol, 1-propanol, 2-propanol, 2-methoxyethanol, 1-butanol, 2-butanol, iso-butyl alcohol, tert-butyl alcohol, 2-ethoxyethanol, diethylene glycol, 1-, 2-, or 3-pentanol, neo-pentyl alcohol, tert-pentyl alcohol, diethylene glycol monomethyl ether, diethylene glycol monoethyl ether, cyclohexanol, benzyl alcohol, phenol, or glycerol. The polar protic solvent can be an alcohol such as methanol, ethanol, 1-propanol, 2-propanol, and the like.

Suitable aprotic solvents can include, by way of example and without limitation, 2-butanone, acetonitrile, dichloromethane, N,N-dimethylformamide (DMF), N,N-dimethylacetamide (DMA), 1,3-dimethyl-3,4,5,6-tetrahydro-2(1H)-pyrimidinone (DMPU), 1,3-dimethyl-2-imidazolidinone (DMI), N-methylpyrrolidinone (NMP), formamide, N-methylacetamide, N-methylformamide, acetonitrile, dimethyl sulfoxide, propionitrile, ethyl formate, methyl acetate, hexachloroacetone, acetone, ethyl methyl ketone, ethyl acetate, sulfolane, N,N-dimethylpropionamide, tetramethylurea, nitromethane, nitrobenzene, hexamethylphosphoramide, and the like.

Suitable hydrocarbon solvents include benzene, cyclohexane, pentane, hexane, toluene, cycloheptane, methylcyclohexane, heptane, ethylbenzene, m-, o-, or p-xylene, octane, indane, nonane, or naphthalene.

The term “reducing agent” as used herein refers to a compound that donates an electron to another compound in a redox reaction. The reducing agent would be oxidized after it loses its electrons. Examples of reducing agents include, but are not limited to, borohydride, triacetoxyborohydride, sodium borohydride, lithium aluminium hydride, hydrogen on palladium, palladium on carbon, and the like.

The term “halogenating agent” as used herein refers to a compound that transfers one or more halogen atoms to the compound with which it is reacting. Examples of halogenating agents include, but are not limited to, trichloromethanesulfonyl chloride, tert-butyl hypochlorite, N-chlorosuccinimide, N-chlorophthalimide, N-bromosuccinimide, N-bromophthalimide, phosphorus tribromide, N-iodosuccinimide, N-iodophthalimide, and the like.

The term “brominating reagent” as used herein refers to a compound that transfers one or more bromine atoms to the compound with which they are reacting. Examples of brominating reagents include, but are not limited to, N-bromosuccinimide, N-bromophthalimide, phosphorus tribromide, and the like.

The term “hydrogenation catalyst” refers to a catalyst suitable to catalyze the reaction between molecular hydrogen and another compound or element (e.g., a hydrogenation reaction). Examples of hydrogenation catalysts include, but are not limited to iridium, nickel, palladium, platinum, rhodium, ruthenium, and the like.

The reactions of the processes described herein can be carried out in air or under an inert atmosphere. Typically, reactions containing reagents or products that are substantially reactive with air can be carried out using air-sensitive synthetic techniques that are well known to the skilled artisan.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like), HPLC, or preparative thin layer chromatography; distillation; sublimation, trituration, or recrystallization. The purity of the compounds, in general, are determined by physical methods such as measuring the melting point (in case of a solid), obtaining a NMR spectrum, or performing a HPLC separation. If the melting point decreases, if unwanted signals in the NMR spectrum are decreased, or if extraneous peaks in an HPLC trace are removed, the compound can be said to have been purified. In some embodiments, the compounds are substantially purified.

The expressions, “ambient temperature” and “room temperature,” as used herein, are understood in the art, and refer generally to a temperature, e.g., a reaction temperature, that is about the temperature of the room in which the reaction is carried out, for example, a temperature from about 20° C. to about 30° C.

### Methods of Use

Compounds of the present disclosure, and salts and solid forms thereof, can inhibit the activity of the FGFR enzyme. For example, compounds of the present disclosure, and salts and solid forms thereof, can be used to inhibit activity of an FGFR enzyme in a cell or in an individual or patient in need of inhibition of the enzyme by administering an inhibiting amount of one or more compounds of the present disclosure, and salts and solid forms thereof, to the cell, individual, or patient. Compounds of the present disclosure, and salts and solid forms thereof, can be used to inhibit activity of the FGFR3 enzyme in a cell or in an individual or patient in need of inhibition of the enzyme by administering an inhibiting amount of one or more, of the present disclosure to the cell, individual, or patient. Compounds of the present disclosure, and salts and solid forms thereof, can be used to inhibit activity of the FGFR2 enzyme in a cell or in an individual or patient in need of inhibition of the enzyme by administering an inhibiting amount of one or more compounds of the present disclosure, and salts and solid forms thereof, to the cell, individual, or patient. Compounds of the present disclosure, and salts and solid forms thereof, can be used to inhibit the activity of an FGFR3 and an FGFR2 enzyme in a cell or in an individual or patient in need of inhibition of the enzyme by administering an inhibiting amount of a compound of the disclosure, or a salt or solid form thereof, to the cell, individual, or patient.

In some embodiments, the compounds of the disclosure, and salts and solid forms thereof, have selective inhibitory activity for the enzyme FGFR3 over FGFR1. In some embodiments, the selectivity of the compounds of the disclosure, and salts and solid forms thereof, for FGFR3 over FGFR1 is 10-fold to 25-fold, or 25-fold to 50-fold. In some embodiments, the compounds of the disclosure, and salts and solid forms thereof, have selective inhibitory activity for the enzyme FGFR3 over FGFR4. In some embodiments, the selectivity of the compounds of the disclosure, and salts and solid forms thereof, for FGFR3 over FGFR4 is 10-fold to 25-fold, 25-fold to 50-fold, or 50-fold to 100-fold. In some embodiments, the compounds of the disclosure, and salts and solid forms thereof, have selective inhibitory activity for the enzyme FGFR3 over FGFR2. In some embodiments, the selectivity of the compounds of the disclosure and salts and solid forms thereof, for FGFR3 over FGFR2 is 1.5-fold to 2-fold, or 2-fold to 3-fold.

In some embodiments, the compounds of the present disclosure, and salts and solid forms thereof, have selective inhibitory activity for the enzyme FGFR3 over FGFR1. Without being bound to a particular theory, it is believed that FGFR1 is associated with certain side effects such as FGFR1-driven hypophosphatemia. Compounds of the present disclosure, and salts and solid forms thereof, can be advantageous over nonselective FGFR inhibitors (e.g., compounds, and salts and solid forms thereof, that have similar inhibitory activity against, for example, both FGFR1 and FGFR3) because the compounds of the present disclosure, and salts and solid forms thereof, have the potential for little or no FGFR1-driven hypophosphatemia side effects, and potentially allow for higher maximum dosage while avoiding side effects associated with FGFR1.

As FGFR inhibitors, the compounds of the present disclosure, and salts and solid forms thereof, are useful in the treatment of various diseases associated with abnormal expression or activity of the FGFR enzyme or FGFR ligands. Compounds, and salts and solid forms thereof, which inhibit FGFR will be useful in providing a means of preventing the growth or inducing apoptosis in tumors, particularly by inhibiting angiogenesis. It is therefore anticipated that compounds of the present disclosure, and salts and solid forms thereof, will prove useful in treating or preventing proliferative disorders such as cancers. In particular, tumors with activating mutants of receptor tyrosine kinases or upregulation of receptor tyrosine kinases may be particularly sensitive to the inhibitors.

In certain embodiments, the disclosure provides a method for treating a FGFR-mediated disorder in a patient in need thereof, comprising the step of administering to said patient a compound, or a salt or solid form thereof, according to the invention, or a pharmaceutically acceptable composition thereof.

In some embodiments, diseases and indications that are treatable using the compounds of the present disclosure, and salts and solid forms thereof, include, but are not limited to hematological cancers, sarcomas, lung cancers, gastrointestinal cancers, genitourinary tract cancers, liver cancers, bone cancers, nervous system cancers, gynecological cancers, and skin cancers.

In some embodiments, cancers that are treatable using the compounds of the present disclosure, and salts and solid forms thereof, are selected from adenocarcinoma, bladder cancer, breast cancer, cervical cancer, cholangiocarcinoma, colorectal cancer, endometrial cancer, esophageal cancer, gall bladder cancer, gastric cancer, glioma, head and neck cancer, hepatocellular cancer, kidney cancer, liver cancer, lung cancer, melanoma, ovarian cancer, pancreatic cancer, prostate cancer, rhabdomyosarcoma, skin cancer, thyroid cancer, leukemia, multiple myeloma, chronic lymphocytic lymphoma, adult T cell leukemia, B-cell lymphoma, acute myelogenous leukemia, Hodgkin's or non-Hodgkin's lymphoma, Waldenstrom's Macroglubulinemia, hairy cell lymphoma, and Burkett's lymphoma.

In some embodiments, cancers that are treatable using the compounds of the present disclosure, and salts and solid forms thereof, are selected from hepatocellular cancer, bladder cancer, breast cancer, cervical cancer, colorectal cancer, endometrial cancer, gastric cancer, head and neck cancer, kidney cancer, liver cancer, lung cancer, ovarian cancer, prostate cancer, esophageal cancer, gall bladder cancer, pancreatic cancer, thyroid cancer, skin cancer, leukemia, multiple myeloma, chronic lymphocytic lymphoma, adult T cell leukemia, B-cell lymphoma, acute myelogenous leukemia, Hodgkin's or non-Hodgkin's lymphoma, Waldenstrom's Macroglubulinemia, hairy cell lymphoma, Burkett's lymphoma, glioblastoma, melanoma, and rhabdosarcoma.

In some embodiments, said cancer is selected from adenocarcinoma, bladder cancer, breast cancer, cervical cancer, cholangiocarcinoma, endometrial cancer, gastric cancer, glioma, head and neck cancer, lung cancer, ovarian cancer, leukemia, and multiple myeloma.

In some embodiments, cancers that are treatable using the compounds of the present disclosure, and salts and solid forms thereof, are selected from hepatocellular cancer, breast cancer, bladder cancer, colorectal cancer, melanoma, mesothelioma, lung cancer, prostate cancer, pancreatic cancer, testicular cancer, thyroid cancer, squamous cell carcinoma, glioblastoma, neuroblastoma, uterine cancer, and rhabdosarcoma.

A cancer characterized by an FGFR2 and/or FGFR3 alteration includes bladder cancers (FGFR3 mutation or fusion), cholangiocarcinoma (FGFR2 fusion) and gastric cancer (FGFR2 amplification).

Compounds of the invention, and salts and solid forms thereof, can be used to treat cancer patients with FGFR2/3 alterations, including mutations, fusion, rearrangement, and amplification. FGFR2/3 alterations were found in a subset of cholangiocarcinoma, urothelial carcinoma, multiple myeloma, gastric adenocarcinoma, glioma, endometrial carcinoma, ovarian carcinoma, cervical cancer, lung cancer and breast cancer. Moreover, the compounds of the invention, and salts and solid forms thereof, can be used to target patients progressing on pan-FGFR inhibitor treatment due to acquirement of gatekeeper mutations (V555M/L/F/I in FGFR3, V564M/L/F/I in FGFR2). Also compounds of the invention, and salts and solid forms thereof, can be used to treat cancer where FGFR2/3 signaling is involved in the resistance to other targeted therapies, for example, it has the potential to overcome resistance to CDK4/6 inhibitors in ER positive breast cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CIVIL), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome), myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma.

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compounds of the present disclosure, and salts and solid forms thereof, can also be useful in the inhibition of tumor metastases.

In addition to oncogenic neoplasms, the compounds of the invention, and salts and solid forms thereof, are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a patient suffering from a skeletal and chondrocyte disorder.

In some embodiments, compounds described herein, and salts and solid forms thereof, can be used to treat Alzheimer's disease, HIV, or tuberculosis.

As used herein, the term “8p11 myeloproliferative syndrome” is meant to refer to myeloid/lymphoid neoplasms associated with eosinophilia and abnormalities of FGFR1.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the FGFR enzyme with a compound described herein, or a salt or solid form thereof, includes the administration of a compound described herein, or a salt or solid form thereof, to an individual or patient, such as a human, having FGFR, as well as, for example, introducing a compound described herein, or a salt or solid form thereof, into a sample containing a cellular or purified preparation containing the FGFR enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, or salts or solid forms thereof, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

It is appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein, or salts or solid forms thereof, for treatment of FGFR-associated diseases, disorders or conditions, or diseases or conditions as described herein. The agents can be combined with the present compounds, or salts or solid forms thereof, in a single dosage form, or the agents can be administered simultaneously or sequentially as separate dosage forms.

Compounds described herein, and salts and solid forms thereof, can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the FGFR inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein, and salts and solid forms thereof, can be used in combination with one or more inhibitors of the enzyme or protein receptors such as HPK1, SBLB, TUT4, A2A/A2B, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein, and salts and solid forms thereof, can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein, and salts and solid forms thereof, can be used in combination with targeted therapies, including JAK kinase inhibitors (Ruxolitinib, additional JAK1/2 and JAK1-selective, baricitinib or INCB39110), Pim kinase inhibitors (e.g., LGH447, INCB053914 and SGI-1776), PI3 kinase inhibitors including PI3K-delta selective and broad spectrum PI3K inhibitors (e.g., INCB50465 and INCB54707), PI3K-gamma inhibitors such as PI3K-gamma selective inhibitors, MEK inhibitors, CSF1R inhibitors (e.g., PLX3397 and LY3022855), TAM receptor tyrosine kinases inhibitors (Tyro-3, Axl, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibitors (e.g., olaparib or rucaparib), inhibitors of BTK such as ibrutinib, c-MET inhibitors (e.g., capmatinib), an ALK2 inhibitor (e.g., INCB00928); or combinations thereof.

For treating cancer and other proliferative diseases, compounds described herein, and salts and solid forms thereof, can be used in combination with chemotherapeutic agents, agonists or antagonists of nuclear receptors, or other anti-proliferative agents. Compounds described herein, and salts and solid forms thereof, can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, CDK2, and CDK4/6 kinase inhibitors such as, for example, those described in WO 2006/056399 can be used in combination with the treatment methods and regimens of the present disclosure for treatment of cancers and solid tumors. Other agents such as therapeutic antibodies can be used in combination with the treatment methods and regimens of the present disclosure for treatment of cancers and solid tumors. The one or more additional pharmaceutical agents can be administered to a patient simultaneously or sequentially.

The treatment methods as disclosed herein can be used in combination with one or more other enzyme/protein/receptor inhibitors therapies for the treatment of diseases, such as cancer and other diseases or disorders described herein. For example, the treatment methods and regimens of the present disclosure can be combined with one or more inhibitors of the following kinases for the treatment of cancer: Akt1, Akt2, Akt3, BCL2, CDK2, CDK4/6, TGF-βR, PKA, PKG, PKC, CaM-kinase, phosphorylase kinase, MEKK, ERK, MAPK, mTOR, EGFR, HER2, HER3, HER4, INS-R, IDH2, IGF-1R, IR-R, PDGFαR, PDGFβR, PI3K (alpha, beta, gamma, delta, and multiple or selective), CSF1R, KIT, FLK-II, KDR/FLK-1, FLK-4, flt-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Non-limiting examples of inhibitors that can be combined with the treatment methods and regimens of the present disclosure for treatment of cancer include an FGFR inhibitor (FGFR1, FGFR2, FGFR3 or FGFR4, e.g., pemigatinib (INCB54828), INCB62079), an EGFR inhibitor (also known as ErB-1 or HER-1; e.g. erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g. olaparib, rucaparib, veliparib or niraparib), a JAK inhibitor (JAK1 and/or JAK2, e.g., ruxolitinib, baricitinib, itacitinib (INCB39110), an LSD1 inhibitor (e.g., INCB59872 and INCB60003), a TDO inhibitor, a PI3K-delta inhibitor (e.g., INCB50465 and INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer), an adenosine receptor antagonist (e.g., A2a/A2b receptor antagonist), an HPK1 inhibitor, a chemokine receptor inhibitor (e.g. CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the treatment methods described herein are combined with administration of a PI3Kδ inhibitor. In some embodiments, the treatment methods described herein are combined with administration of a JAK inhibitor. In some embodiments, the treatment methods described herein are combined with administration of a JAK1 or JAK2 inhibitor (e.g., baricitinib or ruxolitinib). In some embodiments, the treatment methods described herein are combined with administration of a JAK1 inhibitor. In some embodiments, the treatment methods described herein are combined with administration of a JAK1 inhibitor, which is selective over JAK2.

Example antibodies that can be administered in combination therapy include, but are not limited to, trastuzumab (e.g., anti-HER2), ranibizumab (e.g., anti-VEGF-A), bevacizumab (AVASTIN™, e.g., anti-VEGF), panitumumab (e.g., anti-EGFR), cetuximab (e.g., anti-EGFR), rituxan (e.g., anti-CD20), and antibodies directed to c-MET.

One or more of the following agents may be administered to a patient in combination with the treatment methods of the present disclosure and are presented as a non-limiting list: a cytostatic agent, cisplatin, doxorubicin, taxotere, taxol, etoposide, irinotecan, camptostar, topotecan, paclitaxel, docetaxel, epothilones, tamoxifen, 5-fluorouracil, methoxtrexate, temozolomide, cyclophosphamide, SCH 66336, R115777, L778,123, BMS 214662, IRESSA™ (gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The treatment methods and regimens of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds, and salts and solid forms thereof, can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epacadostat, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteosome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure, and salts and solid forms thereof, can be used in combination with one or more other kinase inhibitors including imatinib, particularly for treating patients resistant to imatinib or other kinase inhibitors.

In some embodiments, the treatment methods of the disclosure can be used in combination with a chemotherapeutic in the treatment of cancer, and may improve the treatment response as compared to the response to the chemotherapeutic agent alone, without exacerbation of its toxic effects. In some embodiments, the treatment methods of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining treatment methods of the present disclosure with an additional agent.

The agents can be combined with Compound 1, or a salt or solid form thereof, and/or antibody that binds to human PD-1 or human PD-L1, or antigen-binding fragment thereof, of the present treatment methods in a single or continuous dosage form, or the agents can be administered simultaneously or sequentially as separate dosage forms.

In some embodiments, a corticosteroid such as dexamethasone is administered to a patient in combination with the treatment methods of the disclosure where the dexamethasone is administered intermittently as opposed to continuously.

The treatment methods described herein can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The treatment methods described herein can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the treatment methods and regimens of the present disclosure can be used in combination with tumor specific antigen such as heat shock proteins isolated from tumor tissue itself. In some embodiments, the treatment methods described herein can be combined with dendritic cells immunization to activate potent anti-tumor responses.

The treatment methods and regimens of the present disclosure can be used in combination with bispecific macrocyclic peptides that target Fe alpha or Fe gamma receptor-expressing effectors cells to tumor cells. The treatment methods and regimens of the present disclosure can also be combined with macrocyclic peptides that activate host immune responsiveness.

In some further embodiments, the treatment methods of the disclosure are combined with administration of other therapeutic agents to a patient prior to, during, and/or after a bone marrow transplant or stem cell transplant. The treatment methods and regimens of the present disclosure can be used in combination with bone marrow transplant for the treatment of a variety of tumors of hematopoietic origin.

When more than one pharmaceutical agents is administered to a patient, as discussed in any of the above embodiments, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

In some embodiments, compounds described herein, and salts and solid forms thereof, can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein, and salts and solid forms thereof, can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US 20170107216, US 20170145025, US 20170174671, US 20170174679, US 20170320875, US 20170342060, US 20170362253, and US 20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is MGA012, nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is MGA012 (retifanlimab). In some embodiments, the anti-PD1 antibody is SHR-1210. Other anti-cancer agent(s) include antibody therapeutics such as 4-1BB (e.g. urelumab, utomilumab).

In some embodiments, the compounds of the disclosure, and salts and solid forms thereof, can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the inhibitor of an immune checkpoint molecule is an inhibitor of CD19, e.g., an anti-CD19 antibody. In some embodiments, the anti-CD19 antibody is tafasitamab.

The compounds of the present disclosure, and salts and solid forms thereof, can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure, and salts and solid forms thereof, can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

Compounds of the present disclosure, and salts and solid forms thereof, can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein, and salts and solid forms thereof, can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein, and salts and solid forms thereof, can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, or 10,308,644; U.S. Publ. Nos. 2017/0145025, 2017/0174671, 2017/0174679, 2017/0320875, 2017/0342060, 2017/0362253, 2018/0016260, 2018/0057486, 2018/0177784, 2018/0177870, 2018/0179179, 2018/0179201, 2018/0179202, 2018/0273519, 2019/0040082, 2019/0062345, 2019/0071439, 2019/0127467, 2019/0144439, 2019/0202824, 2019/0225601, 2019/0300524, or 2019/0345170; or PCT Pub. Nos. WO 03042402, WO 2008156712, WO 2010089411, WO 2010036959, WO 2011066342, WO 2011159877, WO 2011082400, or WO 2011161699, which are each incorporated herein by reference in their entirety. In some embodiments, the inhibitor of PD-L1 is INCB086550.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A; also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8H9.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469. In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure, and salts and solid forms thereof, can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1.

In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure, and salts and solid forms thereof, can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196. Inhibitors of arginase inhibitors include INCB1158.

As provided throughout, the additional compounds, inhibitors, agents, etc. can be combined with the present compound, or a salt or solid form thereof, in a single or continuous dosage form, or they can be administered simultaneously or sequentially as separate dosage forms.

In some embodiments, the compounds described herein, and salts and solid forms thereof, can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure, or salts or solid forms thereof, can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable agents for use in combination with compounds described herein, and salts and solid forms thereof, for the treatment of cancer include chemotherapeutic agents, targeted cancer therapies, immunotherapies or radiation therapy. Compounds described herein, and salts and solid forms thereof, may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein, or salts or solid forms thereof. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein, and salts and solid forms thereof, may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein, or salts or solid forms thereof, include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with FGFR inhibitors. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein, or salts or solid forms thereof. In some embodiments, the JAK inhibitor is selective for JAK1 over JAK2 and JAK3.

Other suitable agents for use in combination with compounds described herein, or salts or solid forms thereof, include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein, or salts or solid forms thereof, include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein, or salts or solid forms thereof, include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein, and salts and solid forms thereof, may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure, and salts and solid forms thereof, can be used in combination with bone marrow transplant for the treatment of a variety of tumors of hematopoietic origin.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

As provided throughout, the additional compounds, inhibitors, agents, etc. can be combined with the present compound, or a salt or solid form thereof, in a single or continuous dosage form, or they can be administered simultaneously or sequentially as separate dosage forms.

### Formulation, Dosage Forms and Administration

When employed as pharmaceuticals, the compounds of the present disclosure, or solid forms or salts thereof, can be administered in the form of pharmaceutical compositions. Thus the present disclosure provides a composition comprising the compounds of the present disclosure, or solid forms or salts thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient the compounds, of the present disclosure, or solid forms or salts thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, or a salt or solid form thereof, soft and hard gelatin capsules, suppositories, sterile injectable solutions and sterile packaged powders.

In preparing a formulation, the active compound, or a salt or solid form thereof, can be milled to provide the appropriate particle size prior to combining with the other ingredients. If the active compound, or a salt or solid form thereof, is substantially insoluble, it can be milled to a particle size of less than 200 mesh. If the active compound, or a salt or solid form thereof, is substantially water soluble, the particle size can be adjusted by milling to provide a substantially uniform distribution in the formulation, e.g., about 40 mesh.

The compounds of the present disclosure, or solid forms or salts thereof, may be milled using known milling procedures such as wet milling to obtain a particle size appropriate for tablet formation and for other formulation types. Finely divided (nanoparticulate) preparations of the compounds, or salts or solid forms thereof, of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising a compound of the present disclosure, or a solid form or salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound, or a salt of solid form thereof, may be effective over a wide dosage range and is generally administered in a therapeutically effective amount. It will be understood, however, that the amount of the compound, or a salt or solid form thereof, actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention, or a solid form or salt thereof, can vary according to, e.g., the particular use for which the treatment is made, the manner of administration of the compound, or a salt or solid form thereof, the health and condition of the patient, and the judgment of the prescribing physician. The proportion or concentration of a compound of the disclosure, or a salt or solid form thereof, in a pharmaceutical composition can vary depending upon a number of factors including dosage, chemical characteristics (e.g., hydrophobicity), and the route of administration. For example, the compounds of the invention or a salt or solid form thereof, can be provided in an aqueous physiological buffer solution containing about 0.1 to about 10% w/v of the compound for parenteral administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention, or a salt or solid form thereof. When referring to these preformulation compositions as homogeneous, the active ingredient is typically dispersed evenly throughout the composition so that the composition can be readily subdivided into equally effective unit dosage forms such as tablets, pills and capsules.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds, and salts and solid forms thereof, and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound, or salt or solid form thereof, or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention, or a solid form or salt thereof, can vary according to, e.g., the particular use for which the treatment is made, the manner of administration of the compound, the health and condition of the patient, and the judgment of the prescribing physician. The proportion or concentration of a compound of the invention, or a solid form or salt thereof, in a pharmaceutical composition can vary depending upon a number of factors including dosage, chemical characteristics (e.g., hydrophobicity), and the route of administration. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### EXAMPLES

### Experimental Methods

### X-Ray Powder Diffraction (XRPD)

XRPD was obtained from Bruker D8 Advance ECO X-ray Powder Diffractometer (XRPD) instrument. The general experimental procedures for XRPD were: (1) X-ray radiation from copper at 1.5418 Å and LYNXEYE™ detector; (2) X-ray power at 40 kV, 25 mA; and (3) the sample powder was dispersed on a zero-background sample holder. The general measurement conditions for XRPD were: Start Angle 3 degrees; Stop Angle 30 degrees; Sampling 0.015 degrees; and Scan speed 2 degree/min.

Differential Scanning calorimetry (DSC)

The DSC was obtained from TA Instruments Differential Scanning calorimetry, Discovery DSC2500 with autosampler. The DSC instrument conditions were as follows: 20-300° C. at 10° C./min; Tzero aluminum sample pan and lid; and nitrogen gas flow at 50 mL/min.

### Thermogravimetric Analysis (TGA)

The TGA was obtained from TA Instruments Thermogravimetric Analyzer, Discovery TGA5500 with autosampler. The general experimental conditions for TGA were: ramp from 25° C. to 300° C. at 10° C./min; nitrogen purge gas flow at 25 mL/min; platinum sample holder.

### Example 1. Preparation of Compound 1

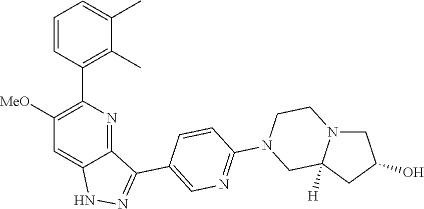 [SMILES: COc1cc2[nH]nc(-c3ccc(N4CCN5C[C@H](O)C[C@H]5C4)nc3)c2nc1-c1cccc(C)c1C]

### Step 1. 1-(2,3-Dimethylphenyl)-2-methoxyethan-1-one

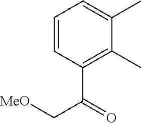

A mixture of magnesium turnings (127.4 g, 5.24 mol, 1.12 equiv) and anhydrous tetrahydrofuran (9.5 L) was heated to 40° C. Once the temperature was stable, iodine (1.5 g, 5.9 mmol, 0.0012 equiv) was added. 1-Bromo-2,3-dimethylbenzene (970 g, 5.24 mol, 1.12 equiv) was added dropwise. After addition of 10% of starting material, the iodine color disappeared. The reaction was exothermic. The dropwise addition continued for 90 minutes and the temperature increased slowly to reflux (65-67° C.). After stirring at 65° C. for one hour, the reaction mixture was cooled in a water bath to room temperature (18° C.). 2-Methoxyacetonitrile (333 g, 4.685 mol, 1 equiv) was added drop-wise over 30 minutes, resulting in an exotherm to 26° C. The reaction mixture was stirred at room temperature overnight. The water bath was replaced with ice/water bath and reaction mixture was cooled to 2° C. Concentrated HCl (37%, 840 mL, 10.1 mol, 2.15 equiv) was mixed with ice (2.2 kg) and water (2.2 L) and the cold solution was added into reaction mixture in one portion. The temperature of the reaction mixture increased to 27° C. After 10 minutes the cooling bath was removed and the reaction mixture was stirred at room temperature for five hours. Ethyl acetate (1200 mL) was added and the layers were separated. The aqueous layer was extracted with additional ethyl acetate (2×600 mL). The combined organic layers were evaporated under reduced pressure. Heptanes (3 L) and silica (500 g) were added to the residue and the mixture was stirred 10 minutes. The solids were filtered and washed with heptanes (2 L) and 10% ethyl acetate in heptanes (5 L). The filtrates were concentrated under reduced pressure. The resulting red oil was further purified on a Biotage 150, eluting initially with a gradient of 0 to 5% ethyl acetate in heptanes, followed by 10% methanol in ethyl acetate. The pure fractions were combined and evaporated under reduced pressure. The oil was further dried on a Büchi rotary evaporator under high vacuum at 40° C. for two hours to give 1-(2,3-dimethylphenyl)-2-methoxyethan-1-one (617.5 g, 74% yield) as yellow oil. LCMS calculated for C₁₁H₁₄O₂: 178.1; Found: 179.1 (M+H⁺). ¹H-NMR (400 MHz, CDCl₃) δ 7.27 (m, 2H), 7.14 (m, 1H), 4.48 (s, 2H). 3.48 (s, 3H), 2.32 (s, 3H), 2.31 (s, 3H).

### Step 2. 1-(tert-butyl)-4-nitro-1H pyrazole

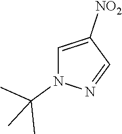

Sulfuric acid (630 ml, 1166 g, 11.89 moles, 1.4 equiv) was added over five minutes to a mixture of 4-nitro-1H-pyrazole (960 g, 8.49 moles, 1 equiv) and tert-butyl acetate (3.1 kg, 3.68 L, 26.68 moles, 3.1 equiv) in 1,4-dioxane (7 L) at room temperature (16° C.). The temperature of the reaction mixture increased to 38° C. during addition. A white precipitate formed in a few minutes. The reaction mixture was warmed to 60° C. and stirred at this temperature for five minutes until all solid dissolved. The reaction mixture was stirred and was allowed to cool to room temperature overnight. LCMS analysis indicated the reaction was complete. The reaction mixture was cooled to 0° C. and the pH was adjusted to 10 with 4 N sodium hydroxide (˜10 L). The product was extracted with ethyl acetate (2×10 L). The combined organic layers were washed with saturated brine (1 L) and dried over sodium sulfate (600 g). The solution was filtered and solvent was evaporated under reduced pressure. The residue was dissolved in heptanes (3 L) at 60° C. The solution was allowed to cool to room temperature with stirring overnight. The solid was filtered, rinsed with heptanes (1 L) and dried under vacuum at 30° C. to give 1-(tert-butyl)-4-nitro-1H-pyrazole (1212 g, 84.4% yield, >97% purity) as a light pink solid. The filtrate was evaporated to dryness. Heptanes (1 L) was added to the residue and the mixture was stirred at room temperature overnight. The solid was filtered, rinsed with heptanes (300 mL) and dried under vacuum at 30° C. to give additional 1-(tert-butyl)-4-nitro-1H-pyrazole (172 g, 12% yield, >97% purity) as light pink solid and the combined yield (1384 g, 96.4% yield). LCMS calculated for C₇H₁₁N₃O₂: 169.1; Found: 170.1 (M+H⁺). ¹H-NMR (400 MHz, CDCl₃) δ 8.23 (s, 1H), 8.08 (s, 1H), 1.62 (s, 9H).

### Step 3. tert-Butyl (1-(tert-butyl)-1H-pyrazol-4-yl)carbamate

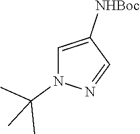 [SMILES: CC(C)(C)OC(=O)Nc1cnn(C(C)(C)C)c1]

A mixture of 1-(tert-butyl)-4-nitro-1H-pyrazole (100 g, 0.591 moles, 1 equiv), di-tert-butyl dicarbonate (142 g, 0.65 moles, 1.1 equiv) and 10% palladium on carbon (5 g, 50% wet) in methanol (1 L) was hydrogenated at 40 psi. The reaction was exothermic and the temperature increased to 45° C. The reaction vessel was evacuated and filled with fresh hydrogen three times to remove carbon dioxide. After the intake of hydrogen slowed down, the reaction was run at room temperature overnight. LCMS analysis indicated the reaction was complete. The reaction mixture was filtered through Celite (200 g), which was rinsed with methanol (2×500 mL). The combined filtrates were concentrated under reduced pressure. The residue was diluted with heptanes (1.5 L) and concentrated to dryness under reduced pressure to remove residual methanol and tert-butanol. The residue was diluted with heptanes (600 mL) and stirred at room temperature overnight. The resulting solids were filtered, rinsed with heptane (2×200 mL) and dried under vacuum at 40° C. to constant weight to give tert-butyl (1-(tert-butyl)-1H-pyrazol-4-yl)carbamate (136 g, 96% yield, 98% purity) as off-white solid. LCMS calculated for C₁₂H₂₁N₃O₂: 239.2; Found: 240.2 (M+H⁺). ¹H-NMR (400 MHz, CDCl₃) δ 7.76 (s, 1H), 7.29 (s, 1H), 6.33 (s, 1H), 1.53 (s, 9H), 1.47 (s, 9H).

### Step 4. tert-Butyl (1-(tert-butyl)-5-formyl-1H-pyrazol-4-yl)carbamate

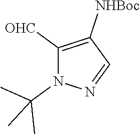 [SMILES: CC(C)(C)OC(=O)Nc1cnn(C(C)(C)C)c1C=O]

2.5 N n-Butyllithium in hexane (2400 mL, 6.0 moles, 2.22 equiv) was added over 30 minutes while maintaining internal temperature below −65° C. to a solution of tert-butyl (1-(tert-butyl)-1H-pyrazol-4-yl)carbamate (647 g, 2.7 moles, 1 equiv) in tetrahydrofuran (9.3 L). After stirring at −76° C. for three hours, anhydrous N,N-dimethylformamide (250 g, 3.42 moles, 1.26 equiv) was added over 3 minutes maintaining internal temperature below −65° C. The reaction mixture was warmed to room temperature and stirred overnight. Saturated ammonium chloride (3100 mL) was added and the reaction mixture was stirred for 15 minutes. The layers were separated and the organic layer was washed with saturated brine (950 mL). The aqueous layers were back-extracted with methyl-tert-butyl ether (2×1.3 L). The combined organic layers were concentrated under reduced pressure. The residue was diluted with heptanes (2.6 L) and concentrated under reduced pressure. The residue was dissolved in a mixture of dichloromethane (400 mL) and heptane (4 L) and was purified over silica gel (3 kg), eluting with a gradient of 0 to 50% ethyl acetate in heptanes. Fractions which contained desired product were combined and concentrated to dryness under reduced pressure. The residue was dried under vacuum at 30° C. overnight to give tert-Butyl (1-(tert-butyl)-5-formyl-1H-pyrazol-4-yl)carbamate (559.4 g, 77.4% yield, 99.5% purity) as light-pink solid. LCMS calculated for C₁₃H₂₁N₃O₃: 267.1; Found: 268.1 (M+H⁺). ¹H-NMR (400 MHz, CDCl₃) δ 10.35 (s, 1H), 8.70 (s, 1H), 7.99 (s, 1H), 1.71 (s, 9H), 1.51 (s, 9H).

### Step 5. Methyl (2S,4R)-4-hydroxypyrrolidine-2-carboxylate hydrochloride

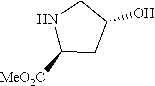

A 22 L flask was charged with (2S,4R)-4-hydroxypyrrolidine-2-carboxylic acid (2450 g 18.68 mol) and methanol (14.5 L) and cooled to 0° C. Thionyl chloride (2434 g, 20.55 mol, 1.1 equiv) was added dropwise over 1 hour with an exotherm steadily increasing to 16° C. The vessel was moved to a heating mantle and heated to reflux for 2 hours. The reactor cooled overnight to room temperature and the reaction was concentrated under reduced pressure. The removal of methanol at the end of the concentration was aided by the addition of tetrahydrofuran (2×500 mL) to give of crude desired compound (3666 g of crude product) as a grey waxy solid. LCMS calculated for C₆H₁₁NO₃: 145.16; Found: 146.21 (M+H⁺). ¹H-NMR (300 MHz, CD₃OD) δ 4.64-4.57 (m, 2H), 3.68 (s, 3H), 3.49-3.43 (m, 1H), 3.34-3.29 (m, 1H), 2.46-2.37 (m, 1H), 2.25-2.16 (m 1H).

### Step 6. Methyl (2S,4R)-1-(2-chloroacetyl)-4-hydroxypyrrolidine-2-carboxylate

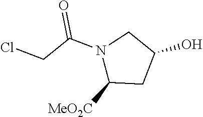 [SMILES: COC(=O)[C@@H]1C[C@@H](O)CN1C(=O)CCl]

A 100 L reactor was charged with methyl (2S,4R)-4-hydroxypyrrolidine-2-carboxylate hydrochloride (2157 g, 11.88 mol) and tetrahydrofuran (65 L). The reactor was cooled to 5° C. Chloroacetyl chloride (1476 g, 13.07 mol, 1.1 equiv) was added over 10 minutes. The reactor was heated to reflux for 3 hours. Analysis by LCMS and NMR indicated the reaction was complete. The reactor was cooled to room temp and the reaction was concentrated under reduced pressure to give the desired compound (2879 g of crude product) as a grey solid. LCMS calculated for C₈H₁₂ClNO₄: 221.64; Found: 222.72 (M+¹H-NMR (300 MHz, CD₃OD) δ 4.54-4.48 (m, 2H), 4.23 (s, 2H), 3.81-3.75 (m, 1H), 3.72 (s, 3H), 3.63-3.57 (m, 1H), 2.32-2.23 (m, 1H), 2.09-2.00 (m, 1H)

### Step 7. (7R,8aS)-2-benzyl-7-hydroxyhexahydropyrrolo[1,2-a]pyrazine-1,4-dione

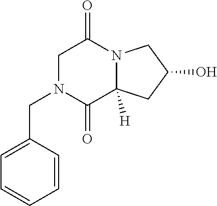 [SMILES: O=C1[C@@H]2C[C@@H](O)CN2C(=O)CN1Cc1ccccc1]

Benzyl amine (2545 g, 23.75 mol, 2 equiv) and triethylamine (1201 g, 11.87 mol, 1 equiv) were sequentially added to a solution of methyl (2S,4R)-1-(2-chloroacetyl)-4-hydroxypyrrolidine-2-carboxylate (2632 g, 11.87 mol) in methanol (10 L) at room temperature. After refluxing for 3 hours, LCMS analysis indicated the reaction was complete. The solution was cooled to room temperature and concentrated under reduced pressure to a brownish solid. The material was dissolved in dichloromethane (4 L) and added to a 50 L separatory funnel containing water (4 L). The layers were separated and the organic layer was washed sequentially with additional water (3×4 L). The organic phase was dried over sodium sulfate (500 g) and concentrated under reduced pressure. The crude material was combined with two other reactions for further purification. The combined material was further purified with trituration in dichloromethane (4 L) at room temperature and filtered. The solids were washed with methyl tert butyl ether (1 L) to provide the desired compound (2845 g) as a tan solid. The filtrates from the trituration were concentrated and purified on a Biotage 150 column eluting with dichloromethane (10 L) followed by 10% methanol in dichloromethane (20 L) to give an additional 1161 g of desired compound (4006 g, 54% yield over 2 steps) as a tan solid. LCMS calculated for C₁₄H₁₆N₂O₃: 260.29; Found: 261.33 (M+H⁺). ¹H-NMR (300 MHz, CD₃OD) δ 7.45-7.20 (m, 5H), 4.62 (s, 2H), 4.62-4.54 (m, 1H), 4.49-4.46 (m, 1H), 4.22-4.15 (m, 1H), 3.77-3.69 (m, 2H), 3.34-3.38 (m, 1H), 2.39-2.32 (m, 1H), 2.19-2.08 (m, 1H).

### Step 8. (7R,8aS)-2-benzyloctahydropyrrolo[1,2-a]pyrazin-7-ol

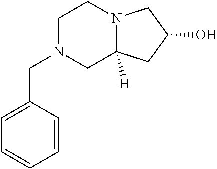 [SMILES: O[C@@H]1C[C@H]2CN(Cc3ccccc3)CCN2C1]

1M Lithium aluminum hydride in THF (11.2 L, 11.2 mol, 6 equiv) was added to a solution of (7R,8aS)-2-benzyl-7-hydroxyhexahydropyrrolo[1,2-c]pyrazine-1,4-dione (486 g, 1.87 mol, 1 equiv) in THF (2.9 L) at −10° C. The reaction mixture was heated to reflux for 16 hours and then cooled −15° C. Ethyl acetate (388 mL) was slowly added to the reaction mixture followed by water (428 mL), 15% sodium hydroxide solution (428 mL) and water (1285 mL). The resulting suspension was stirred at room temperature overnight and filtered through a pad of Celite. The filter cake was washed with THF (2×2 L). The filtrate was concentrated under reduced pressure and co-evaporated with ethyl acetate (2×2 L) to remove the residual THF. The residue was recrystallized at room temperature from ethyl acetate (430 mL). The solid was collected by filtration and washed with cold ethyl acetate (180 mL) to give the desired product (259 g, 59% yield) as a light-yellow solid. LCMS calculated for C₁₄H₂₀N₂O: 232.3; Found: 233.2 (M+H⁺). ¹H-NMR (400 MHz, DMSO-d₆) δ 7.34-7.21 (m, 5H), 4.69 (d, 1H), 4.22-4.12 (m, 1H), 3.53-3.42 (m, 2H), 3.23 (dd, 1H), 2.84-2.73 (m, 2H), 2.67 (dd, 1H), 2.30-2.21 (m, 1H), 2.20-2.12 (m, 1H), 2.09-2.01 (m, 1H), 1.91 (dd, 1H), 1.68 (t, 1H), 1.55-1.42 (m, 2H).

### Step 9. (7R,8aS)-octahydropyrrolo[1,2-a]pyrazin-7-ol

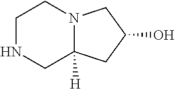

To two nitrogen flushed 2 L Parr shaker bottles was added 10% palladium on carbon (50% wet, 25.6 g, 12.1 mmol, 0.02 equiv). Methanol (50 mL) was added to wet the catalyst. A solution of (7R,8aS)-2-benzyloctahydropyrrolo[1,2-c]pyrazin-7-ol (129 g, 500 mmol, 1 equiv) in methanol (800 mL) was added to each shaker. The reaction mixture was hydrogenated at room temperature at 25 psi of hydrogen for 24 hours. The reaction mixtures were filtered through a pad of celite (150 g) on a sintered glass funnel. The filter cake was washed with methanol (2×450 mL). The filtrate was concentrated under reduced pressure. The residue was diluted with tetrahydrofuran (2×500 mL) and again concentrated each time under reduced pressure to remove the residual methanol. The resulting solid was triturated with THF (360 mL) at room temperature, filtered and washed with tetrahydrofuran (100 mL) to give the desired compound (145 g, 92% yield) as a light-yellow solid. GCMS calculated for C₇H₁₄N₂O: 142.2; Found: 142.1 (M+). ¹H NMR (400 MHz, DMSO-d6): δ 4.69 (s, 1H), 4.06 (dd, J=12.0, 6.2 Hx, 1H), 2.82 (d, J=11.6 Hz, 1H), 2.73 (dd, J=15.8, 9.6 Hz, 3H), 2.57 (t, J=11.6 Hz, 1H) 2.27 (t, J=10.7 Hz, 1H), 2.12-1.96 (m 2H), 1.84 (t, J=10.8 Hz, 1H), 1.66 (d, J=6.1 Hz, 1H), 1.07 (td, J=11.5, 4.1 Hz, 1H). ¹³CNMR (100 MHz, DMSO-d6): δ7.48, 63.85, 61.64, 53.26, 50.62, 45.25, 39.43.

### Step 10. 1-(tert-Butyl)-5-(2,3-dimethylphenyl)-6-methoxy-1H-pyrazolo[4,3-b]pyridine

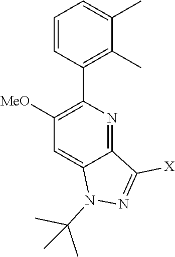

To a 22-liter round-bottom-flask was charged 1-(2,3-dimethylphenyl)-2-methoxyethan-1-one (659.3 gram, 3.70 mole, 1.0 eq), tert-butyl (1-(tert-butyl)-5-formyl-1H-pyrazol-4-yl)carbamate (989.1 g, 3.70 mole, 1.0 eq), and ethanol (3.3 L). The mixture was degassed and refilled with nitrogen. The mixture was heated to 65° C. to a solution. To the solution was slowly added methanesulfonic acid (427 g, 4.44 mole, 1.2 eq) at 65° C. over 1.5 hours. During the acid addition, the mixture turned to orange to red to dark red to brown color. The reaction mixture was agitated at 65° C. for 10 hours to complete the reaction (HPLC showed about 6% of 1-(2,3-dimethylphenyl)-2-methoxyethan-1-one by area and completion consumption of tert-butyl (1-(tert-butyl)-5-formyl-1H-pyrazol-4-yl)carbamate). To the mixture at 65° C. was added water (7.5 L) gradually over 30 minutes. Product 1-(tert-butyl)-5-(2,3-dimethylphenyl)-6-methoxy-1H-pyrazolo[4,3-b]pyridine crystallized gradually out of the solution during the water addition. After the water addition, the mixture was cooled to room temperature (about 20° C.) and agitated for more than 3 hours. The solids were isolated and washed with water (2 times 2 L). The wet cake was dried on the filter by pulling air through the cake overnight (or in vacuum oven at 50-60° C.) to give 1-(tert-Butyl)-5-(2,3-dimethylphenyl)-6-methoxy-1H-pyrazolo[4,3-b]pyridine (99.4 gram, 84% yield, no correction). LCMS calculated for C₁₉H₂₃N₃O: 309. HPLC-MS: 310 (M+H). ¹H NMR (400 MHz, DMSO-d6) δ 8.09 (d, 1H, J=0.9 Hz), 7.67 (d, 1H, J=1.0 Hz), 7.20 (d, 1H, J=7.4 Hz), 7.13 (dd, 1H, J=7.5 Hz), 7.03 (dd, 1H, J=7.5 Hz), 3.87 (s, 3H), 2.29 (s, 3H), 1.93 (s, 3H), 1.76 (s, 9H).

### Step 11. 3-Bromo-1-(tert-butyl)-5-(2,3-dimethylphenyl)-6-methoxy-1H-pyrazolo[4,3-b]pyridine

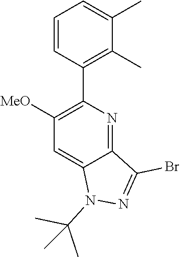 [SMILES: COc1cc2c(nc1-c1cccc(C)c1C)c(Br)nn2C(C)(C)C]

To a 22-liter round-bottom-flask was charged 1-(tert-butyl)-5-(2,3-dimethylphenyl)-6-methoxy-1H-pyrazolo[4,3-b]pyridine (1316.5, 4.15 mole, 1.0 eq) and acetonitrile (10 L). The mixture was heated to 45° C. and N-bromosuccinimide (NBS) (768 gram 4.27 mole, 1.03 eq), was added in eight portions while maintaining the reaction temperature between 45-60° C. The mixture was agitated at about 55° C. for one hour to complete the reaction (HPLC showed 0.1% of 1-(tert-butyl)-5-(2,3-dimethylphenyl)-6-methoxy-1H-pyrazolo[4,3-b]pyridine by area). Water (10 L) was gradually added to the reaction mixture at 45-55° C. Product 3-Bromo-1-(tert-butyl)-5-(2,3-dimethylphenyl)-6-methoxy-1H-pyrazolo[4,3-b]pyridine precipitated out during the water addition. The mixture was cooled to room temperature (about 20° C.) and agitated for over one hour. The solids were isolated. The wet cake was washed with water (2 times 3 L) and dried by pulling air through the cake overnight (or in vacuum oven at 50-60° C.) to give the desired product 3-bromo-1-(tert-butyl)-5-(2,3-dimethylphenyl)-6-methoxy-1H-pyrazolo[4,3-b]pyridine (4147 gram, 97% yield, no correction). Calculated for C₁₉H₂₂BrN₃O: 387, 389. HPLC-MD: 388.2 (M+H), 390.2 (M+H). ¹H NMR (400 MHz, DMSO-d6) δ 7.69 (s, 1H), 7.40 (d, 1H, J=7.5 Hz), 7.16 (dd, 1H, J=7.5 Hz), 7.05 (dd, 1H, J=7.5 Hz, J=1.5 Hz), 3.89 (s, 3H), 2.30 (s, 3H), 1.92 (s, 3H), 1.75 (s 9H).

### Step 12. 1-(tert-Butyl)-5-(2,3-dimethylphenyl)-3-(6-fluoropyridin-3-yl)-6-methoxy-1H-pyrazolo[4,3-b]pyridine

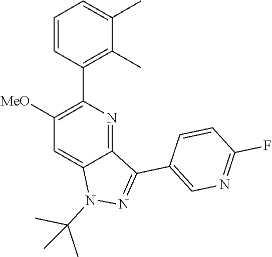 [SMILES: COc1cc2c(nc1-c1cccc(C)c1C)c(-c1ccc(F)nc1)nn2C(C)(C)C]

To a 22-liter round-bottom-flask was charged 3-bromo-1-(tert-butyl)-5-(2,3-dimethylphenyl)-6-methoxy-1H-pyrazolo[4,3-b]pyridine (815 g, 2.06 mole, 1.0 eq), 2-fluoro-5-(4,4,5,5-tetramethyl-1.3.2-diocaborolan-2-yl)pyridine (516 g, 2.27 mole, 1.1 eq), potassium phosphate tribasic monohydrate (1249 g, 5.15 mole, 2.50 eq), 1,4-dioxane (6.6 L) and water (1.63 L). Nitrogen gas was bubbled through the mixture for 5 minutes. Bis(di-tert-butyl(4-dimethylaminophenyl)phosphine)dichloropalladium (II) (Pd-132) (1.459 g, 2.06 mmole, 0.001 eq) was added to the reactor. Nitrogen gas was bubbled through the mixture for 5 minutes. The mixture was heated to reflux under nitrogen and agitated for 1.5 hours to complete the reaction (HPLC showed no detectable 3-bromo-1-(tert-butyl)-5-(2,3-dimethylphenyl)-6-methoxy-1H-pyrazolo[4,3-b]pyridine). The mixture was cooled to 50-65° C. and water (5 L) was added gradually at 50-65° C. to the reaction mixture to precipitate product 1-(tert-butyl)-5-(2,3-dimethylphenyl)-3-(6-fluoropyridin-3-yl)-6-methoxy-1H-pyrazolo[4,3-b]pyridine. The suspension was cooled to room temperature (about 20° C.), agitated for more than 3 hours, and the solids were isolated. The wet cake was washed with water (2×3 L) and dried by pulling air through the cake overnight to give 1-(tert-butyl)-5-(2,3-dimethylphenyl)-3-(6-fluoropyridin-3-yl)-6-methoxy-1H-pyrazolo[4,3-b]pyridine (833 gram, Pd 70.2 ppm). To a 22 liter round-bottom-flask was charged 1-(tert-butyl)-5-(2,3-dimethylphenyl)-3-(6-fluoropyridin-3-yl)-6-methoxy-1H-pyrazolo[4,3-b]pyridine (833 g, Pd 70.2 ppm) and THF (7 L). The mixture was heated to 50° C. Activated charcoal (84 g, 10 wt %) was added to the solution and the mixture was agitated at 50-60° C. for 2 hours. The mixture was cooled to about 35° C. and filtered over Celite bed (250 g). The filter bed was rinsed with THF (1 L) and the combined solution of the filtrate and rinse was heated to about 60° C. Water (10 L) was charged gradually at 50-60° C. to the solution to precipitate 1-(tert-Butyl)-5-(2,3-dimethylphenyl)-3-(6-fluoropyridin-3-yl) methoxy-1H-pyrazolo[4,3-b]pyridine. The suspension was cooled to room temperature (about 20° C.), agitated for more than 3 hours, and the solids were isolated. The wet cake was washed with water (2 L) and dried by pulling air through the cake (or in vacuum oven at 50-60° C.) to give 1-(tert-Butyl)-5-(2,3-dimethylphenyl)-3-(6-fluoropyridin-3-yl)-6-methoxy-1H-pyrazolo[4,3-b]pyridine (818 gram, 98% yield, no correction). Palladium content: 1.8 ppm. Water content by KF: 0.05%. Calculated for C₂₄H₂₅FN₄O: 404.5 HPLC-MS: 405 (M+H). ¹H NMR (400 MHz, DMSO-d6) δ 9.18 (d, 1H, J=7.5 Hz 8.6, J=2.4 Hz), 8.82 (d, 1H, J=2.4 Hz), 7.75 (s, 1H), 7.31 (dd, 1H, J=8.6 Hz, J=2.7 Hz), 7.21-7.09 (m, 3H), 7.13 (dd, 1H), 3.91 (s, 3H), 2.31 (s, 3H), 1.98 (s, 3H), 1.83 (s, 9H).

### Step 13. (7R,8aS)-2-(5-(1-(tert-butyl)-5-(2,3-dimethylphenyl)-6-methoxy-1H-pyrazolo[4,3-b]pyridin-3-yl)pyridin-2-yl)octahydropyrrolo[1,2-a]pyrazin-7-ol

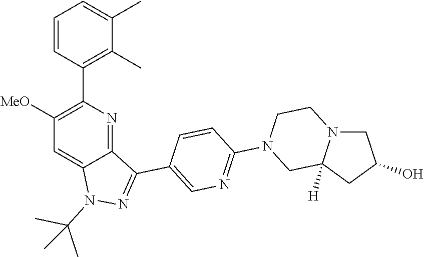 [SMILES: COc1cc2c(nc1-c1cccc(C)c1C)c(-c1ccc(N3CCN4C[C@H](O)C[C@H]4C3)nc1)nn2C(C)(C)C]

In a 22-liter round-bottom-flask was charged (7R,8aS)-octahydropyrrolo[1,2-a]pyrazin-7-ol (168 gram, 1.17 mole, 1.10 eq) and THF (5.65 L). The mixture was heated to 50° C. and then cooled to room temperature (about 20° C., the mixture may be slightly cloudy). n-Butyllithium (2.5 M in hexane, 1.1 kg, 3.96 mole) was charged while maintaining the temperature below 40° C. by applying external cooling. After the addition, the mixture was agitated for 30 minutes at room temperature (about 20° C.) to give a suspension of the dilithium anion.

To another 22-liter round-bottom-flask was charged 1-(tert-butyl)-5-(2,3-dimethylphenyl)-3-(6-fluoropyridin-3-yl)-6-methoxy-1H-pyrazolo[4,3-b]pyridine (435 gram, 1.07 mole, 1.0 eq) and THF (5.65 L). The mixture was heated to 50° C. and then cooled to room temperature (about 20° C.). The THF solution of 1-(tert-butyl)-5-(2,3-dimethylphenyl)-3-(6-fluoropyridin-3-yl)-6-methoxy-1H-pyrazolo[4,3-b]pyridine was added into the dilithium anion suspension at temperature below 35° C. by applying external cooling. After the transfer, the mixture was agitated at room temperature (about 20° C.) for 1 hour to complete the reaction (HPLC showed 1-(tert-butyl)-5-(2,3-dimethylphenyl)-3-(6-fluoropyridin-3-yl)-6-methoxy-1H-pyrazolo[4,3-b]pyridine below 0.2% by area). The mixture was quenched by adding brine (900 mL) below 40° C. The mixture was neutralized by addition of 2M aqueous hydrochloric acid (1.3 L) to pH 8-9 (target pH 8.5). The THF phase was separated and washed with water (2 time 2 L). The organic phase was evaporated by rotavap and the residue was reslurried in acetonitrile (3.2 L) and water (6.4 L) at 50° C. for more than 1 hour to give a suspension. The suspension was cooled to room temperature (about 20° C.) and agitated for 2 hours. The solids were isolated, the wet cake was washed with acetonitrile-water (1/2, v/v) (2 L) and water (2 L), and the solids were dried under vacuum at 70° C. to give (7R,8aS)-2-(5-(1-(tert-butyl)-5-(2,3-dimethylphenyl)-6-methoxy-1H-pyrazolo[4,3-b]pyridin-3-yl)pyridin-2-yl)octahydropyrrolo[1,2-a]pyrazin-7-ol (562 gram, 99% yield, no correction). Calculated for C₃₁H₃₈N₆O₂: 526.7. HPLC-MS: 527.2 (M+H). ¹H NMR (400 MHz, DMSO-d6) δ 9.14 (dd, 1H, J=1.9 Hz), 8.35 (dd, 1H, J=8.9 Hz, J=1.9 Hz), 7.67 (d, 1H, J=1.6 Hz), 7.23 (d, 1H, J=7.5 Hz), 7.17 (d, 1H, J=7.5 Hz), 7.11 (d, 1H, J=7.5 Hz), 6.93 (d, 1H, J=8.9 Hz), 4.79 (dd, 1H, J=4.7 Hz, J=1.6 Hz), 4.41 (dd, 1H, J=12.3, Hz, J=2.8 Hz), 4.27 (t, 1H, J=11.1 Hz), 3.89 (d, 3H, J=1.6 Hz), 3.37-3.30 (m, 2H), 2.97 (d, 1H, J=11.1 Hz), 2.84 (ddd, 1H, J=14 Hz, J=8.0 Hz, J=3.0 Hz), 2.47 (t, 1H, J=11.2 Hz), 2.32 (s, 3H), 2.30-2.14 (m, 2H), 1.99 (s, 3H), 1.99-1.94 (m, 1H), 1.81 (s, 9H), 1.64 (dd, 2H, J=9.4 Hz. J=6.8 Hz).

### Step 14. (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol (Compound 1)

An aqueous sulfuric acid solution was prepared by adding sulfuric acid (1.20 kilogram, 12.2 mole, 12.0 eq) to precooled water (1.07 L). To a five-liter round-bottom-flask was charged (7R,8aS)-2-(5-(1-(tert-butyl)-5-(2,3-dimethylphenyl)-6-methoxy-1H-pyrazolo[4,3-b]pyridin-3-yl)pyridin-2-yl)octahydropyrrolo[1,2-a]pyrazin-7-ol (535 gram, 1.02 mole, 1.0 eq) and the pre-prepared aqueous sulfuric acid solution. Nitrogen gas was bubbled through the solution at more than 1.5 liter per minute. The mixture was heated gradually to 100° C. in 1 hour (note: the reaction temperature was raised slowly to control the rate of the isobutene gas evolution and avoid pressure build-up) and the mixture was agitated at 100° C. for 1 hour. The reaction conversion was about 70% by HPLC. The internal temperature was increased gradually to 105° C. and the mixture was agitated at 105° C. for 1 hour (note: reaction conversion at about 10% by HPLC). The internal temperature was increased gradually from 105° C. to 115° C. over one hour. The mixture was agitated at 115° C. until the reaction completion was confirmed by HPLC ((7R,8aS)-2-(5-(1-(tert-butyl)-5-(2,3-dimethylphenyl)-6-methoxy-1H-pyrazolo[4,3-b]pyridin-3-yl)pyridin-2-yl)octahydropyrrolo[1,2-a]pyrazin-7-ol below 0.5% by HPLC). The mixture was cooled to about 50° C. and water (2.2 L) and THF (535 mL) were charged. The acidic solution was neutralized by addition of 5 M aqueous sodium hydroxide solution to pH 8.0-9.0 (target pH 8.5) while maintaining the internal temperature at 50° C.-60° C. by external cooling (note: (7R,8aS)-2-(5-(5-(2,3-dimethylphenyl)-6-methoxy-1H-pyrazolo[4,3-b]pyridin-3-yl)pyridin-2-yl)octahydropyrrolo[1,2-a]pyrazin-7-ol precipitated out from the mixture gradually during the neutralization). The mixture was agitated at 50° C.-60° C. for more than 2 hours, cooled to room temperature (about 20° C.), and agitated for more than 3 hours. The solids were isolated, the wet cake was washed with water (4 L), and the solids were dried at 70° C. under vacuum to give desired Compound 1 (478 gram, quantitative yield, no correction). Calculated for C₂₇H₃₀N₆O₂: 470.58. HPLC-MS: 471 (M+H). ¹H NMR (400 MHz, DMSO-d6) δ 13.1 (s, 1H), 9.54 (d, 1H, J=2.3 Hz), 8.41 (dd, 1H, J=8.9 Hz, J=2.3 Hz), 7.50 (s, 1H), 7.23 (d, 1H, J=7.4 Hz), 7.17 (d, 1H, J=7.5 Hz), 7.11 (d, 1H, J=7.5 Hz), 6.95 (d, 1H, J=9.0 Hz), 4.82 (d, 1H, J=4.7 Hz), 4.42 (dd, 1H, J=11.7 Hz, J=2.3 Hz), 4.25 (dd, 2H, J=15.6 Hz, J=4.5 Hz), 3.84 (s, 3H), 3.36-3.29 (m, 1H), 3.01-2.93 (m, 1H), 2.84 (dt, 1H, J=12.2 Hz, J=3.3 Hz), 2.46 (d, 1H, J=11.2 Hz), 2.31 (s, 3H), 2.25 (d, 1H, J=9.1 Hz), 2.18 (t, 1H, J=11.5 Hz), 1.97 (s, 3H), 1.64 (dd, 2H, J=9.4 Hz. J=6.5 Hz).

### Example 2. Preparation of Compound 1 Phosphate

In a 22-liter round-bottom-flask was charged Compound 1 (1.52 kg, 3.21 mole, 1.0 eq; from Example 1), activated charcoal (300 gram) and IPA (18 L). The mixture was heated to about 65° C. and agitated at about 65° C. for four hours. The mixture was cooled to about 40° C. and filtered over Celite (500 gram). The filter bed was rinsed with pre-warmed IPA (12 L) at 40° C.-50° C. The combined filtrated was concentrated by rotavap and Compound 1 solids precipitated out during the IPA concentration. The IPA was reduced to about 4 liter and methanol (4 liter) was added to crystallize Compound 1. The solids were isolated and dried to give Compound 1 (1428 gram, 94% yield) as Compound 1 Form II.

In a 22-liter round-bottom-flask was charged Compound 1 (1.42 kilogram, 3.01 mole, 1.0 eq) and THF (15 L). The mixture was heated to about 60° C. Methanol (5 L) was charged to the solution at about 60° C. Phosphoric acid (365 gram, 85%, 3.16 mole, 1.05 eq) in methanol (4.5 L) was charged gradually to the solution at about 60° C. in 1 hour (note: Compound 1 Phosphate precipitated out of the mixture during the phosphoric acid addition). The additional funnel was rinsed with methanol (3 L) into to the reactor. The resulting suspension was agitated at about 60° C. for 3 hours. The mixture was cooled to room temperature (about 20° C.) and agitated for more than 3 hours. The solids were isolated and the wet cake was washed with methanol (3 L). The wet cake and methanol (16 L) was charged back into a 22-liter round-bottom-flask. The mixture was heated to reflux and the methanol (16 L) was distilled out at atmospheric pressure while adding methanol (16 L) gradually to the reactor during the distillation. The mixture was cooled to room temperature (about 20° C.) and agitated for more than 3 hours. The solids were isolated and the wet cake was washed with methanol (2 L). The wet cake and ethanol (12 L) were charged into a 22-liter round-bottom-reactor. The mixture was heated to reflux and agitated for 20 hours under reflux. The mixture was cooled to room temperature (about 20° C.) and agitated for more than 3 hours. The solids were isolated, the wet cake was washed with ethanol (3 L), and the solids were dried at 70-80° C. to give Compound 1 Phosphate Form I (1446 gram, 83% yield). Calculated for C₂₇H₃₀N₆O₂ (Compound 1): 470.58. HPLC-MS: 471 (M+H). ¹H NMR (500 MHz, DMSO-d6) 9.15 (d, 1H, J=2.2 Hz), 8.41 (dd, 1H, J=8.9, 2.3 Hz), 8.1 (br(s), 1H), 7.50 (s, 1H), 7.22 (d, 1H, J=7.4 Hz), 7.16 (t, 1H, J=7.4, 7.4 Hz), 7.10 (d, 1H, J=7.4 Hz), 6.94 (d, 1H, J=9.0 Hz), 4.41 (m, 1H), 4.28 (m, 1H), 4.19 (dd, 1H, J=12.2 Hz), 3.83 (s, 3H), 3.37 (m, 1H), 3.06 (d, 1H, J=12.2 Hz), 2.99 (dd, 1H, J=12.2, 11.0), 2.67 (m, 1H), 2.56 (br (s), 1H), 2.43 (m, 1H), 2.32 (s, 3H), 2.21 (br (s), 1H), 1.97 (s, 3H), 1.71 (m, 1H). ¹³C NMR (125 MHz, DMSO-d6) 157.9, 152.4, 148.0, 145.7, 140.1, 138.6, 137.7, 136.1, 135.1, 134.7, 134.6, 129.2, 127.5, 124.9, 118.2, 106.8, 98.5, 67.5, 62.1, 60.0, 55.7, 49.9, 47.8, 43.4, 38.3, 20.0, 16.2.

### Example 3. Compound 1 Form I

Compound 1 Form I was confirmed as a crystalline solid according to XRPD analysis. The XRPD pattern is shown in FIG. 1 and the peak data are provided in Table 1. Compound I Form I could be prepared as described in Example 1.

The DSC thermogram is shown in FIG. 2. The DSC thermogram revealed an endothermal event at an onset temperature of 22.5° C. with a peak temperature of 70.1° C. and a second endothermal event at an onset temperature of 180.4° C. with a peak temperature of 189.5° C.

The TGA thermogram is shown in FIG. 3. Weight loss of 1.8% mostly due to dehydration was observed below 140° C.

TABLE 1
XRPD Data for Compound 1 Form I

| 2-Theta (°) | H % |
|---|---|
| 4.9 | 6.2 |
| 9.0 | 2.1 |
| 9.3 | 19.7 |
| 10.1 | 2.8 |
| 11.7 | 2.4 |
| 12.3 | 15.6 |
| 13.5 | 0.8 |
| 14.7 | 100 |
| 15.2 | 9.4 |
| 16.3 | 70.6 |
| 16.9 | 9.3 |
| 17.1 | 8.5 |
| 17.8 | 17.0 |
| 18.5 | 6.4 |
| 19.4 | 37.3 |
| 20.5 | 24.0 |
| 21.6 | 13.2 |
| 21.9 | 18.6 |
| 23.4 | 2.7 |
| 24.0 | 10.9 |
| 24.4 | 35.9 |
| 25.1 | 28.7 |
| 25.6 | 11.5 |
| 25.9 | 6.5 |
| 26.7 | 1.3 |
| 27.6 | 0.6 |
| 28.3 | 1.2 |
| 28.8 | 0.9 |
| 29.2 | 0.9 |

### Example 4. Compound 1 Form II

Compound 1 Form II was confirmed as a crystalline solid according to XRPD analysis. The XRPD pattern is shown in FIG. 4 and the peak data are provided in Table 2.

The DSC thermogram is shown in FIG. 5. The DSC thermogram revealed one major endothermal event at an onset temperature of 147.8° C. with a peak temperature of 161.5° C.

The TGA thermogram is shown in FIG. 6. Weight loss of 6.9% mostly due to de-solvation was observed below 180° C. Compound 1 Form II is believed to be a methanol solvate.

TABLE 2
XRPD Data for Compound 1 Form II

| 2-Theta (°) | H % |
|---|---|
| 7.4 | 21.1 |
| 11.1 | 0.6 |
| 11.9 | 15.4 |
| 12.2 | 38.5 |
| 12.5 | 42.4 |
| 12.7 | 62.5 |
| 13.6 | 100 |
| 14.1 | 6.4 |
| 14.5 | 59.0 |
| 14.8 | 1.5 |
| 15.7 | 64.6 |
| 16.3 | 39.7 |
| 16.9 | 46.4 |
| 17.5 | 10.8 |
| 18.1 | 18.6 |
| 18.6 | 6.5 |
| 18.8 | 30.3 |
| 19.1 | 17.6 |
| 19.8 | 32.4 |
| 20.8 | 98.4 |
| 21.3 | 13.0 |
| 22.0 | 11.0 |
| 22.1 | 16.3 |
| 22.9 | 3.7 |
| 23.2 | 95.6 |
| 24.0 | 21.1 |
| 24.3 | 12.0 |
| 24.6 | 2.0 |
| 25.2 | 6.0 |
| 25.7 | 28.1 |
| 25.9 | 80.4 |
| 26.9 | 10.8 |
| 27.4 | 9.3 |
| 27.7 | 4.9 |
| 28.4 | 13.3 |
| 28.9 | 8.7 |
| 29.4 | 8.4 |

### Example 5. Single Crystal Structure Determination of Compound 1 Form II

### Data Collection

A colorless needle having approximate dimensions of 0.39×0.05×0.03 mm³, was mounted on a polymer loop in random orientation. Preliminary examination and data collection were performed on a Rigaku SuperNova diffractometer, equipped with a copper anode microfocus sealed X-ray tube (Cu Kα λ=1.54184 Å) and a Dectris Pilatus3 R 200K hybrid pixel array detector.

Cell constants and an orientation matrix for data collection were obtained from least-squares refinement using the setting angles of 7151 reflections in the range 3.6940°<θ<77.2510°. The space group was determined by the program CRYSALISPRO to be P2₁ (international tables no. 4).

The data were collected to a maximum diffraction angle (2θ) of 155.208° at room temperature. The data are provided below.

| | |
|---|---|
| Empirical formula | C₂₈H₃₄N₆O₃ |
| Formula weight (g mol⁻¹) | 502.61 |
| Temperature (K) | 298(2) |
| Wavelength (Å) | 1.54184 |
| Crystal system | monoclinic |
| Space group | P2₁ |
| Unit cell parameters | |
| a = 7.40169(13) Å | α = 90° |
| b = 7.38781(11) Å | β = 94.3636(15)° |
| c = 23.9007(4) Å | γ = 90° |
| Unit cell volume (Å³) | 1303.15(4) |
| Cell formula units, Z | 2 |
| Calculated density (g cm⁻³) | 1.281 |
| Absorption coefficient (mm⁻¹) | 0.689 |
| F(000) | 536 |
| Crystal size (mm³) | 0.39 × 0.05 × 0.03 |
| Reflections used for cell | 7151 |
| measurement | |
| θ range for cell measurement | 3.6940°-77.2510° |
| Total reflections collected | 13697 |
| Index ranges | −9 ≤ h ≤ 9; −9 ≤ k ≤ |
| | 9; −29 ≤ l ≤ 30 |
| θ range for data collection | θₘᵢₙ = 3.710°, θₘₐₓ = 77.604° |
| Completeness to θₘₐₓ | 98.5% |
| Completeness to θ_{full} = 67.684° | 100% |
| Absorption correction | multi-scan |
| Transmission coefficient range | 0.573-1.000 |
| Refinement method | full matrix least-squares on F² |
| Independent reflections | 5149 [Rᵢₙₜ = 0.0325, R_{σ} = 0.0353] |
| Reflections [I > 2σ(I)] | 4457 |
| Reflections/restraints/parameters | 5149/1/350 |
| Goodness-of-fit on F² | S = 1.05 |
| Final residuals [I > 2σ(I)] | R = 0.0435, R_{w} = 0.1177 |
| Final residuals [all reflections] | R = 0.0507, R_{w} = 0.1236 |
| Largest diff. peak and hole (e Å⁻³) | 0.342, −0.156 |
| Max/mean shift/standard uncertainty | 0.000/0.000 |
| Absolute structure determination | Flack parameter: 0.09(15) |
| | Hooft parameter: 0.15(12) |
| | Friedel coverage: 85.2% |

### Calculated X-ray Powder Diffraction (XRPD) Pattern

A calculated XRPD pattern was generated for Cu radiation using MERCURY and the atomic coordinates, space group, and unit cell parameters from the single crystal structure. Error! Reference source not found.8 shows a calculated XRPD pattern of Compound 1 Form II generated from the single crystal structure.

### Atomic Displacement Ellipsoid and Packing Diagrams

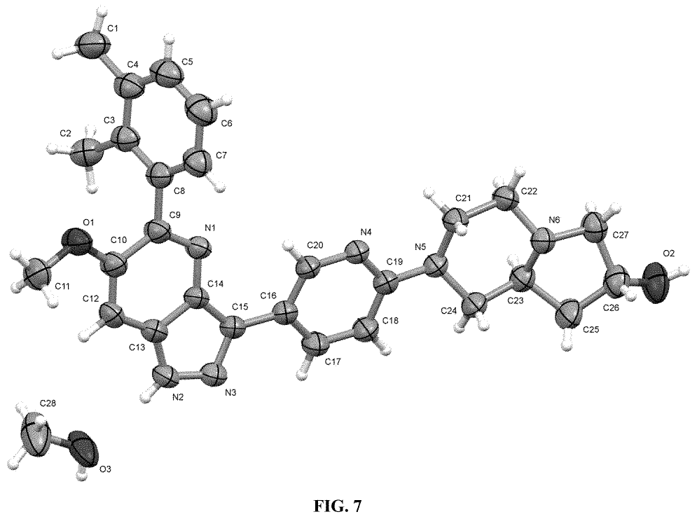

The atomic displacement ellipsoid diagram was prepared using MERCURY. Atoms are represented by 50% probability anisotropic thermal ellipsoids. Packing diagrams and additional figures were generated with MERCURY. Hydrogen bonding is represented as dashed lines. Assessment of chiral centers was performed with PLATON. Absolute configuration is evaluated using the specification of molecular chirality rules. An atomic displacement ellipsoid drawing of Compound 1 Form II is shown in FIG. 7.

### Results & Discussion

An atomic displacement ellipsoid drawing of Compound 1 Form II is shown in FIG. 7. The molecule observed in the asymmetric unit of the single crystal structure is consistent with the proposed molecular structure. The asymmetric unit shown in Error! Reference source not found.7 contains one Compound 1 molecule and one methanol molecule.

The absolute structure can be determined through an analysis of anomalous X-ray scattering by the crystal. Anomalous scattering is assessed through the intensity differences between Friedel pairs. For the reflection data measured up to θₘₐₓ the Friedel coverage was 85.2%. A refined parameter x, known as the Flack parameter, encodes the relative abundance of the two components in an inversion twin. The structure contains a fraction 1−x of the model being refined, and x of its inverse. Provided that a low standard uncertainty is obtained, the Flack parameter should be close to 0 if the solved structure is correct, and close to 1 if the inverse model is correct. The measured Flack parameter for the structure of Compound 1 Form II shown in Error! Reference source not found.7 is 0.09 with a standard uncertainty of 0.15, which indicates sufficient inversion-distinguishing power.

Additional information regarding the absolute structure can be assessed by applying Bayesian statistics to Bijvoet differences. This analysis provides a series of probabilities for different hypotheses of the absolute structure. This analysis results in the Hooft y parameter, which is interpreted in the same fashion as the Flack x parameter. In addition, this analysis results in three probabilities that the absolute structure is either correct, incorrect or a racemic twin. For the current data set the (Flack equivalent) Hooft y parameter is 0.15(12), the probability that the structure is correct is 0.968, the probability that the structure is incorrect is 0.9×10⁻¹⁰ and the probability that the material is a racemic twin is 0.032.

Therefore, the absolute configuration is provided in the model in Error! Reference source not found.7. This structure contains two chiral centers located at C23 and C26 (refer to Error! Reference source not found.7) which bond in the S and R configuration, respectively. Error! Reference source not found.8 shows a calculated XRPD pattern of Compound 1 Form II generated from the single crystal structure.

### Example 6. Characterization of Compound 1 Phosphate Form I

Compound 1 phosphate Form I (the product of Example 2) was confirmed as a crystalline solid according to XRPD analysis. The XRPD pattern is shown in FIG. 9 and the peak data are provided in Table 3.

The DSC thermogram is shown in FIG. 10. The DSC thermogram revealed an endothermal event at an onset temperature of 22.9° C. with a peak temperature of 63.4° C. and a second endothermal event at an onset temperature of 232.0° C. with a peak temperature of 246.2° C.

The TGA thermogram is shown in FIG. 11. Weight loss of 3.1% mostly due to dehydration and 3.2% between 150-275° C. were observed. Weight loss continued at above 275° C. due to decomposition.

TABLE 3
XRPD Data for Compound 1 Phosphate Form I

| 2-Theta (°) | H % |
|---|---|
| 5.3 | 3.3 |
| 8.2 | 94.9 |
| 9.6 | 25.9 |
| 10.0 | 7.7 |
| 10.7 | 5.5 |
| 12.3 | 5.7 |
| 13.0 | 6.2 |
| 13.8 | 55.5 |
| 15.0 | 49.8 |
| 16.1 | 17.6 |
| 16.6 | 34.5 |
| 17.1 | 14.8 |
| 17.6 | 8.0 |
| 18.4 | 7.6 |
| 19.3 | 13.4 |
| 20.1 | 19.6 |
| 21.5 | 3.3 |
| 22.6 | 100 |
| 23.5 | 20.1 |
| 25.2 | 14.1 |
| 26.5 | 6.3 |
| 27.3 | 6.4 |
| 27.8 | 10.3 |

### Example 7. Solubility Measurement of Compound 1 Phosphate

The Compound 1 Phosphate sample was saturated in different solvent systems (Table 4) at 25° C. and 50° C., respectively. Each solubility was measured by HPLC. The results were summarized in Table 4.

At both 25° C. and 50° C., the Compound 1 Phosphate sample has relatively good solubility (>10 mg/mL) in DMF and DMSO. It is slightly soluble (1-10 mg/mL) in MeOH and MeOEtOH, as well as in H₂O at 50° C. It is almost insoluble (<1 mg/mL) in CH₃CN, CHCl₃, DCM, 1,4-dioxane, MIBK, toluene, THF, acetone, n-BuOH, MTBE, EtOH, EtOAc, ethyl formate, heptane, iso-butyl acetate, IPAc, n-PrOH, IPA, and MEK at 25° C. and 50° C., and in H₂O at 25° C.

TABLE 4
Solubility of Compound 1 Phosphate

Solubility

| | 25° C. | 50° C. |
|---|---|---|
| Solvent | (mg/mL) | (mg/mL) |
| CH₃CN | <0.01 | 0.01 |
| CHCl₃ | 0.06 | 0.13 |
| DCM | 0.03 | 0.05 (40° C.) |
| DMF | 14.41 | 19.06 |
| 1,4-dioxane | 0.15 | 0.23 |
| MeOH | 2.26 | 2.44 |
| MeOEtOH | 4.14 | 5.57 |
| MIBK | 0.16 | 0.16 |
| Toluene | <0.01 | 0.02 |
| THF | 0.06 | 0.07 |
| Acetone | 0.07 | 0.10 |
| n-BuOH | 0.36 | 0.47 |
| MTBE | 0.06 | 0.07 |
| DMSO | 33.74 | 44.99 |
| EtOH | 0.53 | 0.68 |
| EtOAc | 0.05 | 0.06 |
| Ethyl formate | 0.12 | 0.14 |
| Heptane | <0.01 | <0.01 |
| iso-Butyl acetate | 0.11 | 0.15 |
| IPAc | 0.02 | 0.03 |
| n-PrOH | 0.40 | 0.40 |
| IPA | 0.23 | 0.28 |
| H₂O | 0.26 | 1.10 |
| 2-Butanone | 0.10 | 0.13 |

### Example 8. Phase Equilibration at 25° C. and 50° C. of Compound 1 Phosphate

Phase equilibration studies were designed to provide information on a predominant crystal form for phase identification. Based on its solubility in various solvent systems (Table 4), Compound 1 Phosphate was equilibrated in the representative groups of solvents at 25° C. (Table 5) and 50° C. (Table 6). To the solvents listed in Table 5 and Table 6, Compound 1 Phosphate was added until a cloudy solution was obtained. Then, about 20 mg of Compound 1 Phosphate was added to the cloudy solution. The mixture was stirred at 25° C. and 50° C. for 48 hours and 24 hours, respectively. The solid was filtered and analyzed by XRPD. The results were listed in Table 5 and Table 6.

The material shows as crystal Form I (which is the same as the starting material Compound 1 Phosphate for phase equilibration at 25° C. and 50° C. in most of the solvents tested). Two new forms (Form II and Form III) were observed in some solvents tested at both temperatures. Form II was observed in DMF (at 25° C. and 50° C.), DMSO (at 25° C. and 50° C.), and THF (at 25° C.). A mixture of Form I and Form II was observed in THF at 50° C. Form III was observed in CH₃CN at 50° C.

TABLE 5
Crystal form for phase equilibration
at 25° C. of Compound 1 Phosphate

| Exp. No. | Solvent | Solid Form |
|---|---|---|
| 1 | N/A | I |
| 2 | CH₃CN | I |
| 3 | CHCl₃ | I |
| 4 | DCM | I |
| 5 | DMF | II |
| 6 | 1,4-Dioxane | I |
| 7 | MeOH | I |
| 8 | MeOEtOH | I |
| 9 | MIBK | I |
| 10 | Toluene | I |
| 11 | THF | II |
| 12 | Acetone | I |
| 13 | n-BuOH | I |
| 14 | MTBE | I |
| 15 | DMSO | II |
| 16 | EtOH | I |
| 17 | EtOAc | I |
| 18 | Ethyl | I |
| | formate | |
| 19 | Heptane | I |
| 20 | iso-Butyl | I |
| | acetate | |
| 21 | IPAc | I |
| 22 | n-PrOH | I |
| 23 | IPA | I |
| 24 | H₂O | amorphous |
| 25 | 2-Butanone | I |

TABLE 6
Crystal form for phase equilibration
at 50° C. of Compound 1 Phosphate

| Exp. No. | Solvent | Solid Form |
|---|---|---|
| 1 | N/A | I |
| 2 | CH₃CN | III |
| 3 | CHCl₃ | I |
| 4 | DCM | I |
| | (40° C.) | |
| 5 | DMF | II |
| 6 | 1,4-Dioxane | I |
| 7 | MeOH | I |
| 8 | MeOEtOH | I |
| 9 | MIBK | I |
| 10 | Toluene | I |
| 11 | THF | I + II |
| 12 | Acetone | I |
| 13 | n-BuOH | I |
| 14 | MTBE | I |
| 15 | DMSO | II |
| 16 | EtOH | I |
| 17 | EtOAc | I |
| 18 | Ethyl formate | I |
| 19 | Heptane | I |
| 20 | iso-Butyl | I |
| | acetate | |
| 21 | IPAc | I |
| 22 | n-PrOH | I |
| 23 | IPA | I |
| 24 | H₂O | amorphous |
| 25 | 2-Butanone | I |

### Example 9. Evaporation at 25° C. and 50° C. of Compound 1 Phosphate

Evaporation studies were carried out to identify the predominant crystal form during uncontrolled precipitation. Experiments that did not result in any particulate solids (i.e. clear thin films and oils) were not studied further. XRPD was used to study the solid state morphology of the crystalline forms of the evaporation samples at 25° C. and 50° C. The results were presented in Table 7 (25° C.) and Table 8 (50° C.).

At 25° C., evaporation experiments in DMF gave crystal Form II and in MeOH produced Form I, the experiments in MeOEtOH, n-BuOH, EtOH, n-PrOH, WA, and H₂O all resulted in amorphous form (Table 7).

TABLE 7
Crystal form identification from evaporation
at 25° C. of Compound I Phosphate

| Exp. No. | Solvent | Solid Form |
|---|---|---|
| 1 | N/A | I |
| 2 | CH₃CN | N/A |
| 3 | CHCl₃ | N/A |
| 4 | DCM | N/A |
| 5 | DMF | II |
| 6 | 1,4-Dioxane | N/A |
| 7 | MeOH | I |
| 8 | MeOEtOH | amorphous |
| 9 | MIBK | N/A |
| 10 | Toluene | N/A |
| 11 | THF | N/A |
| 12 | Acetone | N/A |
| 13 | n-BuOH | amorphous |
| 14 | MTBE | N/A |
| 15 | DMSO | N/A |
| 16 | EtOH | amorphous |
| 17 | EtOAc | N/A |
| 18 | Ethyl formate | N/A |
| 19 | Heptane | N/A |
| 20 | iso-Butyl acetate | N/A |
| 21 | IPAc | N/A |
| 22 | n-PrOH | amorphous |
| 23 | IPA | amorphous |
| 24 | H₂O | amorphous |
| 25 | 2-Butanone | N/A |

N/A: Not available. Ether clear solution or the amount of the precipitate was too small to be analyzed by XRPD.

At 50° C., evaporation experiments in DMF and DMSO produced crystal Form II while all the experiments in MeOH, MeOEtOH, n-BuOH, EtOH, n-PrOH, WA, and H₂O resulted in amorphous form (Table 8).

TABLE 8
Crystal form identification from evaporation
at 50° C. of Compound 1 Phosphate

| Exp. No. | Solvent | Solid Form |
|---|---|---|
| 1 | N/A | I |
| 2 | CH₃CN | N/A |
| 3 | CHCl₃ | N/A |
| 4 | DCM* | N/A |
| 5 | DMF | II |
| 6 | 1,4-Dioxane | N/A |
| 7 | MeOH | amorphous |
| 8 | MeOEtOH | amorphous |
| 9 | MIBK | N/A |
| 10 | Toluene | N/A |
| 11 | THF | N/A |
| 12 | Acetone | N/A |
| 13 | n-BuOH | amorphous |
| 14 | MTBE | N/A |
| 15 | DMSO | II |
| 16 | EtOH | amorphous |
| 17 | EtOAc | N/A |
| 18 | Ethyl formate | N/A |
| 19 | Heptane | N/A |
| 20 | iso-Butyl acetate | N/A |
| 21 | IPAc | N/A |
| 22 | n-PrOH | amorphous |
| 23 | IPA | amorphous |
| 24 | H₂O | amorphous |
| 25 | 2-Butanone | N/A |

*Experiment was performed at 40° C. in DCM

### Example 10. Anti-Solvent Addition of Compound 1 Phosphate

Saturated and almost saturated solutions of Compound 1 Phosphate Form I were prepared in the solvents listed in Table 9 at room temperature, respectively. An anti-solvent was added dropwise to induce precipitation. Experiments that did not produce any particulate solids on anti-solvent addition were not studied further. The results were presented in Table 9.

In anti-solvent addition experiments, a mixture of Form II and amorphous form was formed from DMF/MTBE. Form III was produced from DMF/CH₃CN, MeOEtOH/CH₃CN, and DMSO/CH₃CN. Amorphous form was found in EtOH/Heptane.

TABLE 9
Antisolvent addition of Compound 1 Phosphate

| Exp. No. | Solvent | Anti-solvent | Solid Form |
|---|---|---|---|
| 1 | N/A | N/A | I |
| 2 | DMF | MTBE | II + |
| | | | amorphous |
| 3 | DMF | CH₃CN | III |
| 4 | MeOH | MTBE | N/A |
| 5 | MeOH | CH₃CN | N/A |
| 6 | MeOEtOH | MTBE | N/A |
| 7 | MeOEtOH | CH₃CN | III |
| 8 | DMSO | MTBE | N/A |
| 9 | DMSO | CH₃CN | III |
| 10 | EtOH | Heptane | amorphous |
| 11 | EtOH | MTBE | N/A |
| 12 | EtOH | EtOAc | N/A |
| 13 | H₂O | Acetone | N/A |
| 14 | H₂O | CH₃CN | N/A |

### Example 11. Reverse Addition of Compound 1 Phosphate

Saturated and nearly saturated solutions of Compound 1 Phosphate Form I were prepared in the solvents listed in Table 10 at 30° C. and added dropwise to a larger volume of a miscible anti-solvent. Experiments that did not produce any particulate solids on reverse addition were not studied further. The results were presented in Table 10.

In reverse addition experiments (Table 10), Form II was formed in DMF/MTBE and in DMF/CH₃CN. Form III was produced from MeOH/CH₃CN and MeOEtOH/CH₃CN. A mixture of Form III and amorphous form were observed from DMSO/CH₃CN. A mixture of Form II, Form III, and amorphous form was produced from DMSO/MTBE. An amorphous form was formed in MeOEtOH/MTBE and in EtOH/Heptane.

TABLE 10
Reverse addition of Compound 1 Phosphate

| | | Anti- | |
|---|---|---|---|
| Exp. No. | Solvent | solvent | Solid Form |
| 1 | N/A | N/A | I |
| 2 | DMF | MTBE | II |
| 3 | DMF | CH₃CN | II |
| 4 | MeOH | MTBE | N/A |
| 5 | MeOH | CH₃CN | III |
| 6 | MeOEtOH | MTBE | amorphous |
| 7 | MeOEtOH | CH₃CN | III |
| 8 | DMSO | MTBE | II + III + |
| | | | amorphous |
| 9 | DMSO | CH₃CN | III + |
| | | | amorphous |
| 10 | EtOH | Heptane | amorphous |
| 11 | EtOH | MTBE | N/A |
| 12 | H₂O | Acetone | N/A |
| 13 | H₂O | CH₃CN | N/A |

### Example 12. Quench Cool of Saturated Solution of Compound 1 Phosphate

Saturated solutions of Compound 1 Phosphate prepared at 30° C. were quenched cooled to about −20° C. to −30° C. to induce precipitation of higher energy forms. Representative solvents listed in Table 11 were chosen on the basis of solubility data measured at 25° C. and 50° C. No crystalline solids were produced from the solvents tested.

TABLE 11
Crystal form from quench cool experiment
of Compound 1 Phosphate

| Exp. No. | Solvent | XRPD | Solid Form |
|---|---|---|---|
| 1 | DMF | N/A | N/A |
| 2 | MeOH | N/A | N/A |
| 3 | MeOEtOH | N/A | N/A |
| 4 | EtOH | N/A | N/A |
| 5 | n-PrOH | N/A | N/A |

### Example 13. Crystallization of Saturated Solution with Heating and Cooling Cycles of Compound 1 Phosphate

This experiment was designed to search further for a more stable form than Form I. Saturated solutions of Compound 1 Phosphate were prepared at 50° C., and cooled in a bath slowly by using a programmed circulating bath. Approximately 20 mg of Compound 1 Phosphate with Form I was added to the clear solution to give a slurry. The formed slurry was then heated to 50° C. over 2 hours and then cooled down to 5° C. over 2 hours. This process was repeated for 3 days and the solid was filtered for further analysis. The results were presented in Table 12.

TABLE 12
Crystal form from heating and cooling
cycles of Compound 1 Phosphate

| Exp. No. | Solvent | Solid Form |
|---|---|---|
| 1 | | I |
| 2 | DMF | II |
| 3 | MeOH | I |
| 4 | MeOEtOH | I |
| 5 | EtOH | I |
| 6 | n-PrOH | I |

### Example 14. Compound 1 Phosphate Form II

Compound 1 Phosphate Form II from DMF

Compound 1 Phosphate (301 mg) was mixed with 2 mL of DMF in a 20 mL of vial, then the mixture was stirred at 50° C. for 24 hours, the solid was filtered, air dried and further dried at 50° C. under vacuum overnight.

¹H NMR of the sample showed that it contained about 0.5 mole of DMF. The sample was further dried at 50° C. under vacuum overnight. ¹H NMR of the sample showed it still contains about 0.5 mole of DMF. The XRPD pattern is shown in FIG. 12 and the peak data are provided in Table 13.

The DSC thermogram is shown in FIG. 13. The DSC thermogram revealed an endothermal event at an onset temperature of 137.3° C. with a peak temperature of 151.2° C. and a second endothermal event at an onset temperature of 244.0° C. with a peak temperature of 250.4° C.

The TGA thermogram is shown in FIG. 14. Weight loss of 1.6% between 25-50° C. and weight loss of 9.2% between 100-175° C. were observed. Weight loss continued at above 225° C.

The sample was then slurried in MTBE at room temperature for 4 h, filtered and dried at 50° C. overnight. ¹H NMR of sample indicated that 0.5 mole of DMF was still present.

TABLE 13
XRPD Data for Compound 1 Phosphate Form II from DMF

| 2-Theta | Height | I % |
|---|---|---|
| 3.9 | 123 | 37.2 |
| 6.9 | 331 | 100 |
| 11.1 | 36 | 10.9 |
| 11.6 | 86 | 26 |
| 12.5 | 47 | 14.2 |
| 12.9 | 137 | 41.4 |
| 15.6 | 88 | 26.6 |
| 16.2 | 46 | 13.9 |
| 16.9 | 78 | 23.6 |
| 18.3 | 195 | 58.9 |
| 19.4 | 39 | 11.8 |
| 19.9 | 66 | 19.9 |
| 23.5 | 205 | 61.9 |
| 24.2 | 53 | 16 |
| 26.1 | 38 | 11.5 |
| 26.8 | 80 | 24.2 |

Compound 1 Phosphate Form II from THF

Compound 1 Phosphate Form II was prepared from THF. Compound 1 Phosphate (420 mg) was mixed with 3 mL of THF in a 20 mL of vial, then the mixture was stirred at 25° C. for 48 h, the solid was filtered and dried at 40° C. under vacuum overnight to give 371 mg of powder. ¹H NMR of sample showed that the sample contained 1.25% of THF (w/w). The sample was further dried at 60° C. under vacuum overnight.

¹H NMR of the sample was almost identical to the 41 NMR of the sample prior to drying. It contained 1.25% of THF.

Compound 1 Phosphate Form II from DMSO

Compound 1 Phosphate Form II was prepared from DMSO. Compound 1 Phosphate (1.2 g) was mixed with 2 mL of DMSO in a 20 mL of vial, then the mixture was stirred at 25° C. for 48 hours. The solid was filtered and dried at 40° C. overnight, and then it was further dried at 60° C. under vacuum overnight to afford 511 mg of powder.

¹H NMR of the sample showed that it contained about 38.3% of DMSO.

Overview of Form II Produced from Different Solvents

Table 14 summarized XRPD of the Form II produced from different solvents and drying conditions. The experiments showed that Form II is an unstable form.

TABLE 14
XRPD of Compound 1 Phosphate Form II from
Different Solvents and Drying Conditions

| Exp. No. | Solvent Drying condition | Solid Form |
|---|---|---|
| 1 | N/A | I |
| 2 | DMF | II |
| | (50° C., overnight) | |
| 3 | DMF | II |
| | (50° C., 2 X overnight) | |
| 4 | DMF, | II |
| | (50° C., overnight) | |
| 5 | THF | II |
| | (40° C., overnight) | |
| 6 | THF | II |
| | (60° C., overnight) | |
| 7 | DMSO | II |
| | (40° C., overnight) | |
| 8 | DMSO | II |
| | (60° C., overnight) | |

### Example 15. Compound 1 Phosphate Form III

Compound 1 Phosphate (155 mg) was mixed with 3 mL of CH₃CN in a 20 mL of vial, then the mixture was stirred at 50° C. for 24 hours. The solid was filtered and dried at 50° C. under vacuum for 30 hours to afford 142 mg of powder.

The XRPD pattern is shown in FIG. 15 and the peak data are provided in Table 15.

The DSC thermogram is shown in FIG. 16. The DSC thermogram revealed an endothermal event at an onset temperature of 193.0° C. with a peak temperature of 202.8° C.

The TGA thermogram is shown in FIG. 17. Weight loss of 2.3% up to 150° C., 3.3% between 150-250° C. and 3.9% between 250-300° C. were observed. Weight loss continued at above 300° C.

The data characterizing Compound 1 Phosphate Form III is consistent with an acetonitrile solvate.

TABLE 15
XRPD Data for Compound 1 Phosphate Form III

| 2-Theta | Height | I % |
|---|---|---|
| 3.9 | 123 | 45.6 |
| 5.0 | 156 | 57.8 |
| 5.7 | 52 | 19.3 |
| 8.1 | 55 | 20.4 |
| 12.4 | 72 | 26.7 |
| 14.0 | 79 | 29.3 |
| 16.2 | 156 | 57.8 |
| 17.0 | 57 | 21.1 |
| 18.5 | 48 | 17.8 |
| 20.3 | 63 | 23.3 |
| 22.5 | 270 | 100 |
| 24.2 | 41 | 15.2 |

### Example 16

### The Stability Relationship of Compound 1 Phosphate Forms

The relative stability of the three forms (Forms I, II, and III) as well as amorphous form of Compound 1 Phosphate studied and compared by phase equilibration of mixture experiments in EtOH and in a mixture solvent of EtOH/H₂O (9/1). As summarized in Table 16, Compound 1 Phosphate (Form I) was mixed and slurred with other forms in EtOH and in a mixed solvent of EtOH/H₂O (9/1) at 50° C. for 6 h respectively. XRPD analysis showed that the mixtures were transformed to Form I in both cases.

TABLE 16
XRPD Results of Phase Equilibration of Form Mixtures

| | | | Product |
|---|---|---|---|
| | Starting Material | | Solid |
| Exp. No. | Form (ID) | Solvent | Form |
| 1 | I | EtOH | I |
| | II | | |
| | III | | |
| | Amorphous | | |
| 2 | I | EtOH/H₂O | I |
| | II | (9/1) | |
| | III | | |
| | Amorphous | | |

### Example 17. Preparation of Compound 1 Hydrochloride Salt Form I

Compound 1 (85.45 mg) was dissolved in 2 mL of 1:1 dichloromethane (DCM)/methanol in a 4 mL clear glass vial with stirring. To the solution, 33.3 μL of 6 M aqueous hydrochloric acid (1.1 eq) was added and mixed well. The solution was evaporated without a cover at room temperature to provide an oil overnight. To the resulting oil, 2 mL of acetone was added and slurried at 65-70° C. for 1-2 h. The suspension was stirred at room temperature for 1 h. The hydrochloride salt was collected by filtration, washed with acetone and vacuum dried at 50° C. for 1 h. The salt ratio between hydrochloric acid and Compound 1 was determined to be 1.53 by ion chromatography analysis.

The hydrochloride salt was confirmed as a crystalline solid according to XRPD analysis. The XRPD pattern is shown in FIG. 18 and the peak data are provided in Table 17.

The DSC thermogram is shown in FIG. 19. The DSC thermogram revealed an endothermal event at an onset temperature of 96.4° C. with a peak temperature of 124.0° C. and a second endothermal event at an onset temperature of 186.8° C. with a peak temperature of 203.6° C.

The TGA thermogram is shown in FIG. 20. Weight loss of 8.3% below 125° C. and 2.3% between 125-215° C. were observed. Weight loss continued above 215° C. due to decomposition of the compound.

TABLE 17
XRPD Data for Compound 1 Hydrochloride Salt Form I

| 2-Theta (°) | H % |
|---|---|
| 5.0 | 100 |
| 6.4 | 23.5 |
| 7.8 | 25.2 |
| 8.1 | 2.0 |
| 10.1 | 73.0 |
| 10.8 | 1.8 |
| 12.0 | 1.9 |
| 12.8 | 7.0 |
| 13.5 | 27.4 |
| 14.5 | 19.1 |
| 14.7 | 22.5 |
| 15.1 | 77.5 |
| 15.7 | 38.8 |
| 16.2 | 27.5 |
| 16.7 | 20.9 |
| 17.5 | 23.2 |
| 17.8 | 8.8 |
| 18.4 | 9.4 |
| 19.1 | 3.6 |
| 19.8 | 30.9 |
| 20.1 | 9.4 |
| 21.0 | 38.8 |
| 21.8 | 6.4 |
| 22.7 | 4.3 |
| 23.1 | 5.4 |
| 23.4 | 10.8 |
| 24.0 | 99.4 |
| 24.5 | 32.7 |
| 25.2 | 87.3 |
| 25.8 | 14.7 |
| 26.2 | 46.0 |
| 26.4 | 48.8 |
| 26.8 | 12.1 |
| 27.5 | 24.1 |
| 28.3 | 12.3 |
| 29.3 | 6.3 |

### Example 18. Preparation of Compound 1 Hydrochloride Salt Form II

Compound 1 (101.88 mg) was dissolved in 2 mL of 1:1 dichloromethane (DCM)/methanol in a 4 mL clear glass vial with stirring. To the solution, 6 M aqueous hydrochloric acid (79.5 μL, 2.2 eq) was added and mixed well. The solution was evaporated without a cap at room temperature overnight to provide an oil. To the resulting oil, acetone (2 mL) was added and slurried at 65-70° C. for 1-2 h. The suspension was stirred at room temperature for 1 h. The hydrochloride salt was collected by filtration, washed with acetone and vacuum dried at 50° C. for 1 h. The salt ratio between hydrochloric acid and Compound 1 was determined to be 2.13 by ion chromatography analysis.

The hydrochloride salt was confirmed as a crystalline solid according to XRPD analysis. The XRPD pattern was shown in FIG. 21 and the peak data were provided in Table 18.

The DSC thermogram is shown in FIG. 22. The DSC thermogram revealed an endothermal event at an onset temperature of 94.8° C. with a peak temperature of 137.1° C. and a second endothermal event at an onset temperature of 195.2° C. with a peak temperature of 229.8° C.

The TGA thermogram is shown in FIG. 23. Weight loss of 10.8% below 150° C. and 2.9% between 150-220° C. were observed. Weight loss continued above 220° C. due to decomposition of the compound.

TABLE 18
XRPD Data for Compound I Hydrochloride Salt Form II

| 2-Theta (°) | H % |
|---|---|
| 4.4 | 27.7 |
| 5.5 | 3.8 |
| 6.6 | 40.3 |
| 7.0 | 24.5 |
| 9.0 | 32.7 |
| 11.1 | 36.8 |
| 12.3 | 3.6 |
| 13.1 | 33.2 |
| 13.5 | 32.4 |
| 13.8 | 61.2 |
| 14.6 | 8.3 |
| 14.8 | 57.7 |
| 15.3 | 46.4 |
| 16.0 | 7.5 |
| 16.4 | 24.0 |
| 16.7 | 24.8 |
| 17.1 | 15.2 |
| 17.3 | 12.2 |
| 18.1 | 43.7 |
| 18.3 | 26.1 |
| 19.3 | 12.0 |
| 20.1 | 11.4 |
| 20.3 | 12.7 |
| 20.6 | 37.0 |
| 20.8 | 9.1 |
| 21.3 | 10.3 |
| 21.8 | 10.2 |
| 21.9 | 12.4 |
| 22.4 | 22.5 |
| 22.7 | 26.8 |
| 23.7 | 44.0 |
| 24.0 | 34.2 |
| 24.8 | 75.0 |
| 25.3 | 34.6 |
| 25.7 | 100 |
| 26.2 | 40.9 |
| 26.9 | 29.3 |
| 27.2 | 26.7 |
| 27.8 | 14.8 |
| 28.3 | 32.6 |
| 29.1 | 13.1 |
| 29.5 | 20.1 |

### Example 19. Preparation of Compound 1 L-Tartrate Salt

Compound 1 (98.06 mg) was dissolved in THF (1 mL), methanol (1 mL) and dichloromethane (DCM) (1 mL) in a 4 mL clear glass vial and heated at 70° C. with stirring. After the solution was cooled to room temperature, L-tartaric acid (33.76 mg, 1.21 eq) was added and mixed well. The solution was evaporated without a cap at room temperature overnight to provide a solid. To the resulted solid, acetone (2 mL) was added and stirred at room temperature for 2 h. The L-tartrate salt was collected by filtration, washed with acetone and vacuum dried at 50° C. for 1 h. The salt ratio between L-tartaric acid and free base was determined to be 1.1 by NMR analysis.

The L-Tartrate salt was confirmed as a crystalline solid according to XRPD analysis. The XRPD pattern is shown in FIG. 24 and the peak data are provided in Table 19.

The DSC thermogram is shown in FIG. 25. The DSC thermogram revealed an endothermal event at an onset temperature of 31.2° C. with a peak temperature of 70.4° C. and a second endothermal event at an onset temperature of 119.3° C. with a peak temperature of 129.1° C.

The TGA thermogram is shown in FIG. 26. Weight loss of 5.6% below 150° C. and 21.2% between 150-300° C. were observed.

TABLE 19
XRPD Data for Compound 1 L-Tartrate Salt

| 2-Theta (°) | H % |
|---|---|
| 9.0 | 8.6 |
| 11.7 | 75.9 |
| 12.0 | 10.7 |
| 12.6 | 49.8 |
| 13.9 | 100 |
| 15.2 | 80.4 |
| 15.6 | 72.3 |
| 17.0 | 45.9 |
| 17.2 | 9.5 |
| 18.0 | 12.4 |
| 18.5 | 69.6 |
| 19.0 | 22.3 |
| 19.5 | 41.0 |
| 19.9 | 44.3 |
| 20.2 | 32.7 |
| 20.6 | 9.6 |
| 21.2 | 2.8 |
| 21.8 | 83.7 |
| 22.8 | 33.5 |
| 23.1 | 6.2 |
| 23.8 | 82.3 |
| 24.6 | 27.5 |
| 24.8 | 25.6 |
| 25.4 | 27.3 |
| 26.1 | 40.1 |
| 26.4 | 10.4 |
| 27.3 | 25.0 |
| 28.0 | 17.4 |
| 28.4 | 19.4 |
| 29.0 | 13.8 |
| 29.3 | 9.2 |

### Example 20. Preparation of Compound 1 Malonate Salt

Compound 1 (75.53 mg) was dissolved in 3 mL of 2:1 tetrahydrofuran (THF)/methanol in a 4 mL clear glass vial and heated at 70° C. to dissolve with stirring. After the solution was cooled to room temperature, malonic acid (20.17 mg, 1.24 eq) was added and mixed well. The solution was evaporated without a cap at room temperature overnight to provide an oil. To the resulted oil, acetone (2 mL) was added to solid out and stirred at room temperature for 2 h. The malonate salt was collected by filtration, washed with acetone and vacuum dried at 50° C. for 1 h. The salt ratio between malonic acid and Compound 1 was determined to be 1.2 by NMR analysis.

The malonate salt was confirmed as a crystalline solid according to XRPD analysis. The XRPD pattern is shown in FIG. 27 and the peak data are provided in Table 20.

The DSC thermogram is shown in FIG. 28. The DSC thermogram revealed an endothermal event at an onset temperature of 22.2° C. with a peak temperature of 56.8° C. and a second endothermal event at an onset temperature of 164.3° C. with a peak temperature of 173.3° C.

The TGA thermogram is shown in FIG. 29. Weight loss of 16.9% between 50-200° C. was observed.

TABLE 20
XRPD Data for Malonate Salt

| 2-Theta (°) | H % |
|---|---|
| 4.0 | 19.1 |
| 4.6 | 13.2 |
| 6.7 | 1.4 |
| 8.1 | 7.4 |
| 9.0 | 14.0 |
| 9.2 | 6.1 |
| 10.4 | 7.8 |
| 11.1 | 3.5 |
| 11.7 | 12.8 |
| 12.2 | 2.7 |
| 13.1 | 24.0 |
| 13.9 | 27.6 |
| 14.0 | 25.4 |
| 14.4 | 5.7 |
| 15.1 | 13.5 |
| 15.7 | 13.0 |
| 16.3 | 13.3 |
| 17.1 | 100 |
| 17.9 | 67.5 |
| 18.2 | 11.5 |
| 18.5 | 12.6 |
| 18.8 | 75.2 |
| 19.4 | 21.8 |
| 19.6 | 14.1 |
| 19.9 | 11.8 |
| 20.7 | 34.1 |
| 21.2 | 8.4 |
| 22.4 | 29.7 |
| 22.7 | 61.1 |
| 23.1 | 5.1 |
| 23.8 | 12.8 |
| 24.2 | 12.6 |
| 24.5 | 23.3 |
| 24.9 | 16.8 |
| 25.2 | 14.9 |
| 26.1 | 5.7 |
| 27.2 | 21.9 |
| 28.0 | 15.7 |
| 28.2 | 10.3 |
| 29.0 | 7.4 |

### Example 21. Preparation of Compound 1 Mesylate Salt

Compound 1 (86.54 mg) was dissolved in 3 mL of 2:1 tetrahydrofuran (THF)/methanol in a 4 mL clear glass vial and heated at 70° C. to dissolve with stirring. After the solution was cooled to room temperature, 14.4 μL of methanesulfonic acid (1.2 eq) was added and mixed well. The solution was evaporated without a cap at room temperature overnight to provide an oil. To the resulting oil, acetone (2 mL) was added to solid out and stirred at room temperature for 2 h. The mesylate salt was collected by filtration, washed with acetone and vacuum dried at 50° C. for 1 h. The salt ratio between methanesulfonic acid and Compound 1 was determined to be 2.4 by NMR analysis.

The mesylate salt was confirmed as a crystalline solid according to XRPD analysis. The XRPD pattern is shown in FIG. 30 and the peak data are provided in Table 21.

The DSC thermogram is shown in FIG. 31. The DSC thermogram revealed an endothermal event at an onset temperature of 41.4° C. with a peak temperature of 92.6° C. and a second endothermal event at an onset temperature of 167.3° C. with a peak temperature of 177.9° C.

The TGA thermogram is shown in FIG. 32. Weight loss of 5.0% below 150° C. and 7.0% between 150-300° C. were observed.

TABLE 21
XRPD Data for Mesylate Salt

| 2-Theta (°) | H % |
|---|---|
| 4.9 | 70.3 |
| 5.7 | 26.8 |
| 7.5 | 8.3 |
| 8.0 | 100 |
| 9.9 | 20.1 |
| 10.3 | 4.3 |
| 11.1 | 13.3 |
| 11.5 | 6.0 |
| 11.8 | 20.6 |
| 12.4 | 4.0 |
| 13.1 | 8.2 |
| 14.1 | 3.3 |
| 14.3 | 6.0 |
| 14.9 | 3.0 |
| 15.9 | 7.6 |
| 17.7 | 12.3 |
| 18.4 | 9.1 |
| 19.2 | 12.0 |
| 19.6 | 25.2 |
| 20.0 | 27.8 |
| 20.6 | 34.9 |
| 22.2 | 37.1 |
| 23.3 | 9.0 |
| 23.5 | 11.2 |
| 24.2 | 20.2 |
| 25.9 | 12.5 |
| 26.3 | 10.0 |
| 27.6 | 16.5 |
| 29.0 | 4.2 |
| 29.5 | 3.1 |

### Example 22. Other Salts

Additional salts were prepared, as set forth in the below table, and were observed to be amorphous.

| Salts | Solid Sate |
|---|---|
| Adipic acid salt of Compound 1 | Amorphous |
| Fumaric acid salt of Compound 1 | Amorphous |
| Maleic acid salt of Compound 1 | Amorphous |
| Malic acid salt of Compound 1 | Amorphous |
| Succinic acid salt of Compound 1 | Amorphous |
| Trifluoroacetic acid salt of Compound 1 | Amorphous |

### Example 23. Alternative Synthesis of Compound 1

An alternative synthesis, which relies on the use of a Boc protecting group, is provided below.

### Step 1. 5-(2,3-dimethylphenyl)-6-methoxy-1Hpyrazolo[4,3-b]pyridine

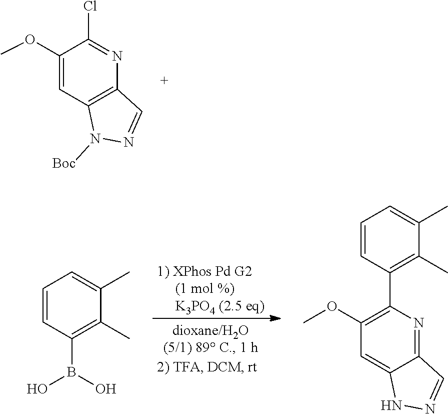

A stirred mixture of tert-butyl 5-chloro-6-methoxy-1H-pyrazolo[4,3-b]pyridine-1-carboxylate (20.00 g, 68.4 mmol) (from Ambeed), (2,3-dimethylphenyl)boronic acid (15.70 g, 103 mmol) (from Combi-Blocks, 1.5 eq), XPhos Pd G2 (0.538 g, 0.684 mmol) (1 mol %) and Potassium phosphate tribasic monohydrate (40.6 g, 171 mmol) (2.5 eq) in 1,4-Dioxane (200 ml) (10 volume) and Water (40 ml) (2 volume) at room temperature was degassed with house vacuum and refilled with Nitrogen three times. It was heated at reflux (89° C.) for 1 h. LCMS showed the reaction was done cleanly.

The reaction mixture was cooled to room temperature, diluted with DCM (200 mL) and water (100 mL). The DCM layer was separated, dried over Na2SO4, filtered and concentrated in vacuo to give the crude product, tert-butyl 5-chloro-6-methoxy-1H-pyrazolo[4,3-b]pyridine-1-carboxylate as a yellow foamy solid.

To a stirred solution of the above crude product in DCM (50 mL) at rt was added trifluoroacetic acid (76 ml, 1026 mmol) (15 eq). The reaction mixture was stirred at rt for 2 h. LCMS showed the N-Boc deprotection was done.

The reaction mixture was concentrated in vacuo to remove the DCM. The residue was cooled in an ice bath, then slowly basified with 4 N NaOH (200 mL) to pH 14. The resulting slurry was stirred at rt for 1 h.

The solid was collected by vacuum filtration and washed with water. The wet cake was then washed with IPA until the filtrate turn into light yellow. It was dried at rt under house vacuum overnight to give the desired product, 5-(2,3-dimethylphenyl)-6-methoxy-1Hpyrazolo[4,3-b]pyridine, as a light yellow solid (17.0 g, 95.46% pure by HPLC @ 220 nm, 94% yield for two steps). LCMS: 254.2 (M+H⁺).

### Step 2. tert-butyl 5-(2,3-dimethylphenyl)-3-iodo-6-methoxy-1H-pyrazolo[4,3-b]pyridine carboxylate

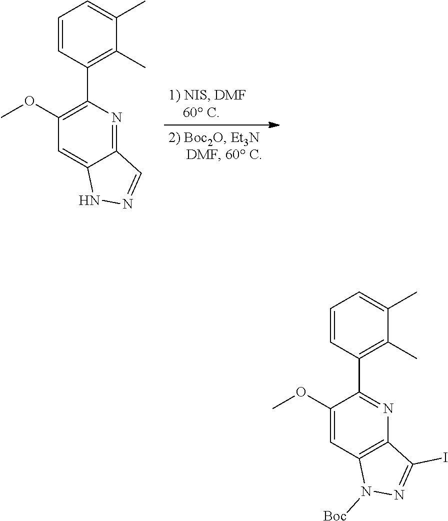

To a stirred solution of 5-(2,3-dimethylphenyl)-6-methoxy-1H-pyrazolo[4,3-b]pyridine (16.8 g, 63.3 mmol) (95.46% pure by HPLC @ 220 nm) in DMF (100 ml) (6 volume) at rt was added niodosuccinimide (17.99 g, 76 mmol) (1.2 eq). The resulting red solution was heated at 60° C. for 1 h 30 min. LCMS showed the reaction was done.

The reaction mixture was cooled to rt. Di-tert-butyl dicarbonate (22.04 ml, 95 mmol) (1.5 eq) and Triethylamine (17.74 ml, 127 mmol) (2 eq) was then added. The reaction mixture was heated at 60° C. for 1 h. LCMS showed the N-Boc protection was done.

The reaction mixture was cooled to rt, water (200 mL) was slowly added to precipitate the product. The resulting slurry was stirred at rt for 1 h. The solid was collected by vacuum filtration and washed with water. The wet cake was then washed with IPA (about 60 mL) until the filtrate turn into light yellow. It was dried at rt under house vacuum overnight to give the desired product, tert-butyl 5-(2,3-dimethylphenyl)-3-iodo-6-methoxy-1H-pyrazolo[4,3-b]pyridine-1-carboxylate, as a light yellow solid (26.2 g, 98.80% pure by HPLC @ 220 nm, 85% yield for two steps). LCMS: 480.2 (M+H⁺).

### Step 3. tert-butyl 5-(2,3-dimethylphenyl)-3-(6-fluoropyridin-3-yl)-6-methoxy-1Hpyrazolo[4,3-b]pyridine-1-carboxylate

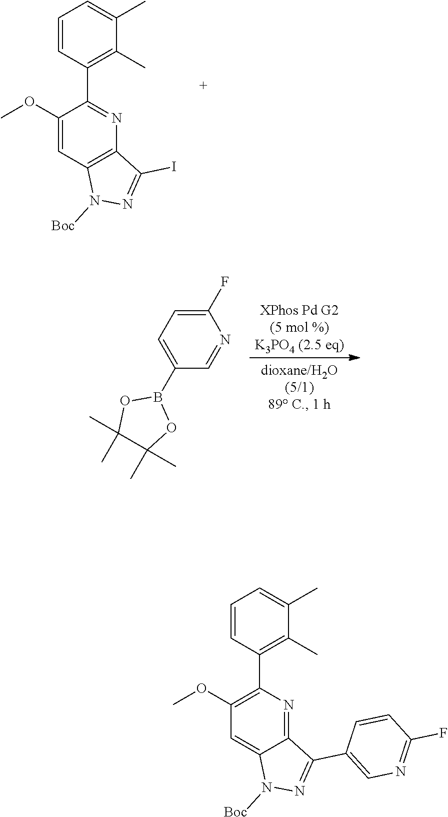 [SMILES: COc1cc2c(nc1-c1cccc(C)c1C)c(I)nn2C(=O)OC(C)(C)C ; CC1(C)OB(c2ccc(F)nc2)OC1(C)C ; COc1cc2c(nc1-c1cccc(C)c1C)c(-c1ccc(F)nc1)nn2C(=O)OC(C)(C)C]

A stirred mixture of tert-butyl 5-(2,3-dimethylphenyl)-3-iodo-6-methoxy-1H-pyrazolo[4,3-b]pyridine-1-carboxylate (20.00 g, 41.4 mmol) (99.21% pure by HPLC @ 220 nm), 2-fluoro-5-(4,4,5,5-tetramethyl-1,3,2-dioxaborolan-2-yl)pyridine (14.58 g, 62.1 mmol) (1.5 eq), Xphos Pd G2 (1.629 g, 2.070 mmol) (5 mol %) and Potassium phosphate tribasic monohydrate (25.09 g, 103 mmol) (2.5 eq) in 1,4-Dioxane (200 ml) (10 volume) and Water (40 ml) (2 volume) at rt was degassed with house vacuum and refilled with Nitrogen three times. It was then heated at reflux (89° C.) for 1 h. LCMS and HPLC (254 nm) showed the reaction was done with 79.0% of the desired product, 8.4% of N-Boc deprotected product and 2.06% of the des-Iodo side-product.

The reaction mixture was cooled to rt, water (200 mL) was added to precipitate the product. The resulting slurry was stirred at rt for 1 h. The solid was collected by vacuum filtration and washed with water. The wet cake was washed with IPA (about 10 mL). It was treated with DCM (500 mL). The resulting suspension was treated with 10 wt % aqueous N-acetylcystein (with 1/1 mole ratio or 13 wt % of K₃PO₄, 200 mL). It was heated at 40° C. for 1 h, then at rt for 1 h. The DCM layer was separated, washed with water (2×200 mL). The DCM layer was separated, concentrated in vacuo. The resulting thick slurry was treated with MeOH (400 mL). It was concentrated in vacuo to remove about 210 mL of MeOH. The resulting slurry was stirred at rt for 10 min. The solid was collected by vacuum filtration and washed with MeOH (about 20 mL). It was dried at rt under house vacuum overnight to give the desired product, tert-butyl 5-(2,3-dimethylphenyl)-3-(6-fluoropyridin-3-yl)-6-methoxy-1Hpyrazolo[4,3-b]pyridine-1-carboxylate, as an off-white solid [17.5 g, 98.67% pure by HPLC @ 220 nm, 93% yield). LCMS: 449.3 (M+H⁺). The residual Pd content was 217 ppm by XRF.

### Step 4. (7R,8aS)-2-(5-(5-(2,3-dimethylphenyl)-6-methoxy-1Hpyrazolo[4,3-b]pyridin-3-yl)pyridin-2-yl)octahydropyrrolo[1,2-a]pyrazin-7-ol

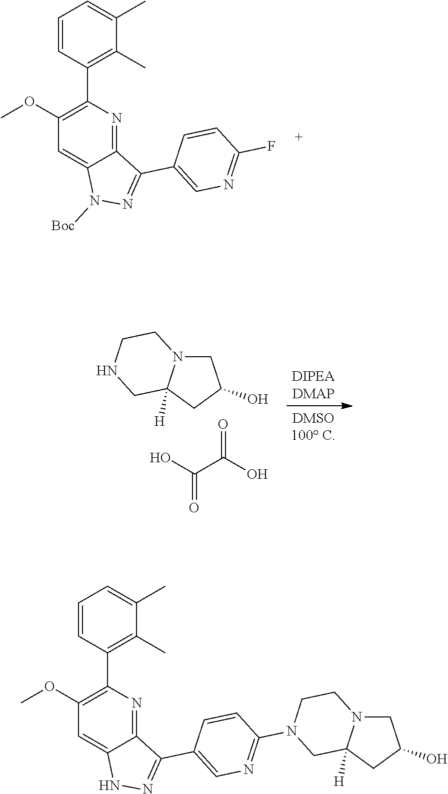

To a stirred suspension of tert-butyl 5-(2,3-dimethylphenyl)-3-(6-fluoropyridin-3-yl) methoxy-1Hpyrazolo[4,3-b]pyridine-1-carboxylate (0.100 g, 0.220 mmol) (98.67% pure by HPLC at 220 nm) and (7R,8aS)-octahydropyrrolo[1,2-a]pyrazin-7-ol oxalate (0.051 g, 0.220 mmol) (1 eq) in DMSO (1 ml) (10 volume) at rt was added N,N-Diisopropylethylamine (0.146 ml, 0.880 mmol) (4 eq) and 4-(Dimethylamino)pyridine (5.49 mg, 0.044 mmol) (0.2 eq). The reaction mixture was heated at 100° C. for 21 h. It became a clear solution within 15 min at 100° C. LCMS and HPLC (254 nm) showed there were 46.8% of the desired product, and 47.9% of the Boc-deprotected starting material.

### Step 5. (7R,8aS)-2-(5-(5-(2,3-dimethylphenyl)-6-methoxy-1Hpyrazolo[4,3-b]pyridin-3-yl)pyridin-2-yl)octahydropyrrolo[1,2-a]pyrazin-7-ol

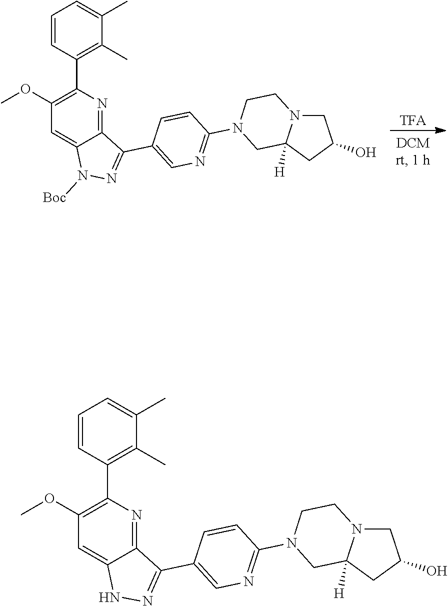 [SMILES: COc1cc2c(nc1-c1cccc(C)c1C)c(-c1ccc(N3CCN4C[C@H](O)C[C@H]4C3)nc1)nn2C(=O)OC(C)(C)C ; COc1cc2[nH]nc(-c3ccc(N4CCN5C[C@H](O)C[C@H]5C4)nc3)c2nc1-c1cccc(C)c1C ; "TFA"]

To a stirred suspension of tert-butyl 5-(2,3-dimethylphenyl)-3-(6-((7R,8aS)-7-hydroxyhexahydropyrrolo[1,2-a]pyrazin-2(1H)-yl)pyridin-3-yl)-6-methoxy-1H-pyrazolo[4,3-b]pyridine-1-carboxylate (3.158 g, 5.48 mmol) (98.97% pure by HPLC @ 254 nm) in dichloromethane (12.6 ml) (4 volume) at rt was added trifluoroacetic acid (10.17 ml, 137 mmol) (25 eq). The resulting solution was stirred at rt for 1 h. LCMS and HPLC showed the reaction was done with 96.20% of the desired product (B) and 2.05% of its isomer impurity. The reaction mixture was basified with 3 N NaOH (ca. 25 mL, exothermic) in an ice bath to pH 11. The resulting slurry was stirred at rt for 1 h. The solid was collected by vacuum filtration and washed with water to give the crude product as a light brown solid which was further purified by reslurry in IPA (26 mL, 10 volume vs the product) at reflux (oil bath temperature 90° C.) for 15 min. It was then slowly cooled to rt and stirred at rt for 1 h. The solid was collected by vacuum filtration and washed with IPA. It was dried at rt under house vacuum overnight to give the product as a light yellow solid (1.318 g, 96.23% pure with 3.18% of the isomer by HPLC @ 220 nm, 49.2% yield). LCMS: 471.3 (M+H⁺). The filtrate was concentrated in vacuo. The residue was purified by reslurry in EtOAc (12 mL) at reflux (oil bath temperature 90° C.) for 15 min. It was then slowly cooled to rt and stirred at rt for 1 h. The solid was collected by vacuum filtration and washed with 1/1 EtOAc/MTBE. It was dried at rt under house vacuum overnight to give the product as a light yellow solid (0.540 g, 97.28% pure with 1.26% of the isomer by HPLC @ 220 nm, 20.4% yield). LCMS: 471.3 (M+H⁺). The filtrate was concentrated in vacuo. The residue was purified by Biotage Isolera (with 40 g silica gel column) eluting with 0-30% MeOH/DCM to give the desired product as a light yellow foamy solid (0.750 g, 98.93% pure with 0.83% of the isomer by HPLC @ 220 nm, 28.8% yield). LCMS: 471.3 (M+H⁺). Total product: 2.608 g, 98.4% total yield.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A solid form of Compound 1 having the formula: 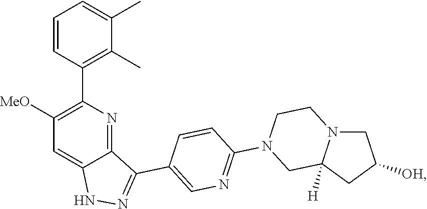 [SMILES: COc1cc2[nH]nc(-c3ccc(N4CCN5C[C@H](O)C[C@H]5C4)nc3)c2nc1-c1cccc(C)c1C]
wherein the solid form is crystalline.

2. The solid form of claim 1, wherein the solid form is Compound 1 Form I.

3. The solid form of claim 2, having at least one characteristic X-ray powder diffraction (XRPD) peak selected from about 4.9, about 9.3, about 12.3, about 14.7, and about 16.3 degrees 2-theta.

4. The solid form of claim 2, having at least one characteristic XRPD peak selected from about 4.9, about 9.3, about 12.3, about 14.7, about 16.3, about 17.8, about 19.4, about 20.5, about 21.9, about 24.4, and about 25.1 degrees 2-theta.

5. The solid form of claim 2, having an XRPD pattern with characteristic peaks as substantially shown in FIG. 1.

6. The solid form of claim 2, which exhibits a DSC thermogram having endotherm peaks at temperatures of about 70° C. and about 190° C.

7. The solid form of claim 2, having a DSC thermogram substantially as depicted in FIG. 2.

8. The solid form of claim 2, having a TGA thermogram substantially as depicted in FIG. 3.

9. The solid form of claim 1, wherein the solid form is a methanol solvate.

10. The solid form of claim 9, wherein the solid form is Compound 1 Form II.

11. The solid form of claim 9, having at least one characteristic XRPD peak selected from about 7.4, about 12.7, about 13.6, about 20.8, and about 23.2 degrees 2-theta.

12. The solid form of claim 9, having at least one characteristic XRPD peak selected from about 7.4, about 12.5, about 12.7, about 13.6, about 14.5, about 15.7, about 16.9, about 20.8, about 23.2, and about 25.9 degrees 2-theta.

13. The solid form of claim 9, having an XRPD pattern with characteristic peaks as substantially shown in FIG. 4.

14. The solid form of claim 9, which exhibits a DSC thermogram having an endotherm peak at a temperature of about 162° C.

15. The solid form of claim 9, having a DSC thermogram substantially as depicted in FIG. 5.

16. The solid form of claim 9, having a TGA thermogram substantially as depicted in FIG. 6.

17. A salt which is an acid addition salt of Compound 1, having the structure: 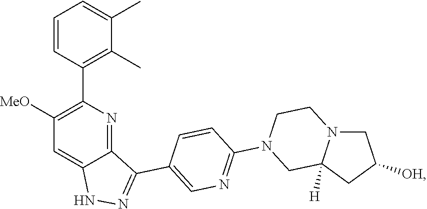 [SMILES: COc1cc2[nH]nc(-c3ccc(N4CCN5C[C@H](O)C[C@H]5C4)nc3)c2nc1-c1cccc(C)c1C]
wherein the acid is phosphoric acid, hydrochloric acid, L-tartaric acid, malonic acid, methanesulfonic acid, adipic acid, fumaric acid, maleic acid, malic acid, or succinic acid.

18. The salt of claim 17, wherein the acid is phosphoric acid.

19. The salt of claim 18, wherein the salt is crystalline.

20. The salt of claim 19, wherein the salt is Compound 1 Phosphate Form I.

21. The salt of claim 20, having at least one characteristic XRPD peak selected from about 8.2, about 9.6, about 13.8, about 15.0 and about 22.6 degrees 2-theta.

22. The salt of claim 20, having at least one characteristic XRPD peak selected from about 8.2, about 9.6, about 13.8, about 15.0, about 16.1, about 16.6, about 18.4, about 19.3, about 20.1, and about 22.6 degrees 2-theta.

23. The salt of claim 20, having an XRPD pattern with characteristic peaks as substantially shown in FIG. 9.

24. The salt of claim 20, which exhibits a DSC thermogram having endotherm peaks at temperatures of about 63° C. and about 246° C.

25. The salt of claim 20, having a DSC thermogram substantially as depicted in FIG. 10.

26. The salt of claim 20, having a TGA thermogram substantially as depicted in FIG. 11.

27. The salt of claim 19, wherein the salt is Compound 1 Phosphate Form II.

28. The salt of claim 27, having at least one characteristic XRPD peak selected from about 3.9, about 6.9, about 12.9, about 18.3, and about 23.5 degrees 2-theta.

29. The salt of claim 27, having at least one characteristic XRPD peak selected from about 3.9, about 6.9, about 11.6, about 12.9, about 15.6, about 16.9, about 18.3, about 23.5 and about 26.8 degrees 2-theta.

30. The salt of claim 27, having an XRPD pattern with characteristic peaks as substantially shown in FIG. 12.

31. The salt of claim 27, which exhibits a DSC thermogram having endotherm peaks at temperatures of about 151° C. and about 250° C.

32. The salt of claim 27, having a DSC thermogram substantially as depicted in FIG. 13.

33. The salt of claim 27, having a TGA thermogram substantially as depicted in FIG. 14.

34. The salt of claim 19, wherein the salt is an acetonitrile solvate.

35. The salt of claim 19, wherein the salt is Compound 1 Phosphate Form III.

36. The salt of claim 35, having at least one characteristic XRPD peak selected from about 3.9, about 5.0, about 16.2, and about 22.5 degrees 2-theta.

37. The salt of claim 35, having at least one characteristic XRPD peak selected from about 3.9, about 5.0, about 5.7, about 8.1, about 12.4, about 14.0, about 16.2, about 17.0, about 20.3 and about 22.5 degrees 2-theta.

38. The salt of claim 35, having an XRPD pattern with characteristic peaks as substantially shown in FIG. 15.

39. The salt of claim 25, which exhibits a DSC thermogram having an endotherm peak at a temperature of about 203° C.

40. The salt of claim 35, having a DSC thermogram substantially as depicted in FIG. 16.

41. The salt of claim 35, having a TGA thermogram substantially as depicted in FIG. 17.

42. The salt of claim 17, wherein the acid is hydrochloric acid.

43. The salt of claim 42, wherein the salt is crystalline.

44. The salt of claim 43, wherein the salt is of Compound 1 Hydrochloride Form I.

45. The salt of claim 44, having at least one characteristic XRPD peak selected from about 5.0, about 6.4, about 7.8, about 10.1, about 15.1, and about 24.0 degrees 2-theta.

46. The salt of claim 44, having at least one characteristic XRPD peak selected from about 5.0, about 6.4, about 7.8, about 10.1, about 15.1, about 15.7, about 19.8, about 21.0, about 24.0, about 25.2, about 26.2, and about 26.4 degrees 2-theta.

47. The salt of claim 44, having an XRPD pattern with characteristic peaks as substantially shown in FIG. 18.

48. The salt of claim 44, which exhibits a DSC thermogram having endotherm peaks at temperatures of about 124° C. and about 204° C.

49. The salt of claim 44, having a DSC thermogram substantially as depicted in FIG. 19.

50. The salt of claim 44, having a TGA thermogram substantially as depicted in FIG. 20.

51. The salt of claim 43, wherein the salt is of Compound 1 Hydrochloride Form II.

52. The salt of claim 51, having at least one characteristic XRPD peak selected from about 4.4, about 6.6, about 7.0, about 9.0, about 11.1, and about 13.8 degrees 2-theta.

53. The salt of claim 51, having at least one characteristic XRPD peak selected from about 4.4, about 6.6, about 7.0, about 9.0, about 11.1, about 13.8, about 14.8, about 15.3, about 18.1, about 23.7, about 24.8, about 25.7, and about 26.2 degrees 2-theta.

54. The salt of claim 51, having an XRPD pattern with characteristic peaks as substantially shown in FIG. 21.

55. The salt of claim 51, which exhibits a DSC thermogram having endotherm peaks at temperatures of about 137° C. and about 230° C.

56. The salt of claim 51, having a DSC thermogram substantially as depicted in FIG. 22.

57. The salt of claim 51, having a TGA thermogram substantially as depicted in FIG. 23.

58. The salt of claim 17, wherein the acid is an L-tartaric acid.

59. The salt of claim 58, wherein the salt is crystalline.

60. The salt of claim 59, having at least one characteristic XRPD peak selected from about 11.7, about 13.9, about 15.2, about 21.8, and about 23.8 degrees 2-theta.

61. The salt of claim 59, having at least one characteristic XRPD peak selected from about 11.7, about 12.6, about 13.9, about 15.2, about 15.6, about 17.0, about 18.5, about 21.8, and about 23.8 degrees 2-theta.

62. The salt of claim 59, having an XRPD pattern with characteristic peaks as substantially shown in FIG. 24.

63. The salt of claim 59, which exhibits a DSC thermogram having endotherm peaks at temperatures of about 70° C. and about 129° C.

64. The salt of claim 59, having a DSC thermogram substantially as depicted in FIG. 25.

65. The salt of claim 59, having a TGA thermogram substantially as depicted in FIG. 26.

66. The salt of claim 17, wherein the acid is a malonic acid.

67. The salt of claim 66, wherein the salt is crystalline.

68. The salt of claim 67, having at least one characteristic XRPD peak selected from about 4.0, about 9.0, about 13.9, about 17.1, about 17.9, about 18.8, and about 22.7 degrees 2-theta.

69. The salt of claim 67, having at least one characteristic XRPD peak selected from about 4.0, about 9.0, about 13.1, about 13.9, about 14.0, about 17.1, about 17.9, about 18.8, about 20.7, and about 22.7 degrees 2-theta.

70. The salt of claim 67, having an XRPD pattern with characteristic peaks as substantially shown in FIG. 27.

71. The salt of claim 67, which exhibits a DSC thermogram having endotherm peaks at temperatures of about 57° C. and about 173° C.

72. The salt of claim 67, having a DSC thermogram substantially as depicted in FIG. 28.

73. The salt of claim 67, having a TGA thermogram substantially as depicted in FIG. 29.

74. The salt of claim 17, wherein the acid is a methanesulfonic acid.

75. The salt of claim 74, wherein the salt is crystalline.

76. The salt of claim 75, having at least one characteristic XRPD peak selected from about 4.9, about 5.7, about 8.0, about 9.9, and about 22.2 degrees 2-theta.

77. The salt of claim 75, having at least one characteristic XRPD peak selected from about 4.9, about 5.7, about 8.0, about 9.9, about 11.8, about 19.6, about 20.0, about 20.6, and about 22.2 degrees 2-theta.

78. The salt of claim 75, having an XRPD pattern with characteristic peaks as substantially shown in FIG. 30.

79. The salt of claim 75, which exhibits a DSC thermogram having endotherm peaks at temperatures of about 93° C. and about 178° C.

80. The salt of claim 75, having a DSC thermogram substantially as depicted in FIG. 31.

81. The salt of claim 75, having a TGA thermogram substantially as depicted in FIG. 32.

82. A process for preparing Compound 1 having the formula: 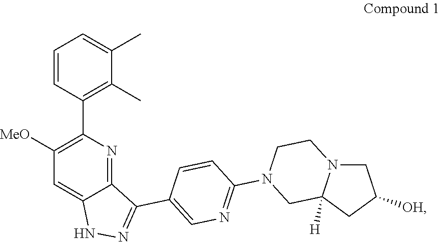 [SMILES: COc1cc2[nH]nc(-c3ccc(N4CCN5C[C@H](O)C[C@H]5C4)nc3)c2nc1-c1cccc(C)c1C]
or a salt thereof, comprising:
deprotecting Compound 2 having the formula: 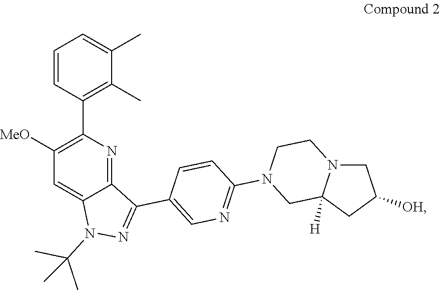 [SMILES: COc1cc2c(nc1-c1cccc(C)c1C)c(-c1ccc(N3CCN4C[C@H](O)C[C@H]4C3)nc1)nn2C(C)(C)C]
or a salt thereof, with A1, wherein A1 is an acid.

83. The process of claim 82, wherein A1 is an inorganic acid.

84. The process of claim 82, wherein A1 is sulfuric acid.

85. The process of claim 82, wherein the deprotecting is performed in the presence of S1, wherein S1 is a protic solvent.

86. The process of claim 85, wherein S1 is water.

87. The process of claim 82, wherein Compound 2 or a salt thereof is prepared by a process comprising: contacting Compound 3 having the formula: 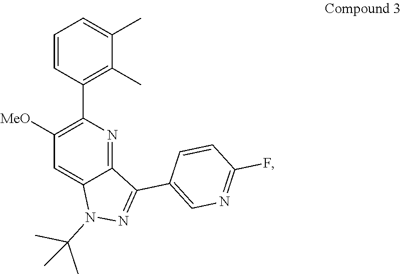 [SMILES: COc1cc2c(nc1-c1cccc(C)c1C)c(-c1ccc(F)nc1)nn2C(C)(C)C]
or a salt thereof, with Compound 4 having the formula: 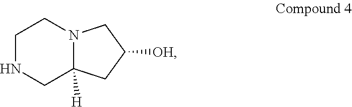
or a salt thereof, in the presence of B1, wherein B1 is a base.

88. The process of claim 87, wherein B1 is an organolithium base.

89. The process of claim 87, wherein B1 is n-butyllithium.

90. The process of claim 87, wherein the contacting is performed in the presence of S2, wherein S2 is a polar aprotic solvent.

91. The process of claim 90, wherein S2 is tetrahydrofuran.

92. The process of claim 87, wherein Compound 3 or a salt thereof is prepared by a process comprising: coupling Compound 5 having the formula: 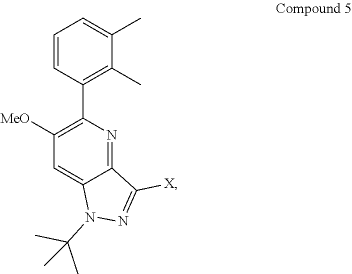
or a salt thereof, wherein X is halo, with Compound 6 having the formula: 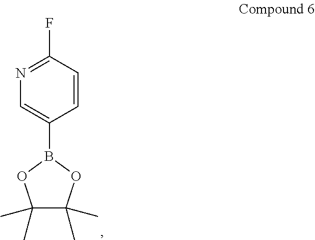
or a salt thereof, in the presence of CA1 and B2, wherein CA1 is a catalyst and B2 is a base.

93. The process of claim 92, wherein CA1 is a palladium catalyst.

94. The process of claim 92, wherein CA1 is bis(di-tert-butyl(4-dimethylaminophenyl)phosphine)dichloropalladium (II) (Pd-132).

95. The process of claim 92, wherein B2 is an inorganic base.

96. The process of claim 92, wherein B2 is potassium phosphate tribasic monohydrate.

97. The process of claim 92, wherein X is Br.

98. The process of claim 92, wherein Compound 5 or a salt thereof is prepared by a process comprising: halogenating Compound 7 having the formula: 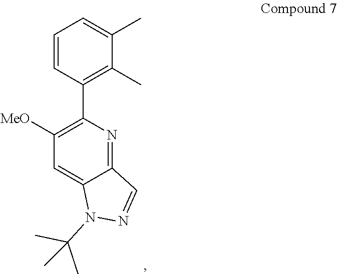 [SMILES: COc1cc2c(cnn2C(C)(C)C)nc1-c1cccc(C)c1C]
or a salt thereof, in the presence of H1, wherein H1 is a halogenating agent.

99. The process of claim 98, wherein H1 is a brominating reagent.

100. The process of claim 98, wherein H1 is N-bromosuccinimide.

101. The process of claim 98, wherein Compound 7 or a salt thereof is prepared by a process comprising: contacting Compound 8 having the formula: 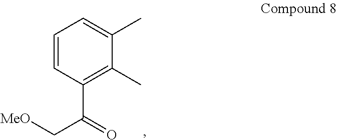
or a salt thereof, with Compound 9 having the formula: 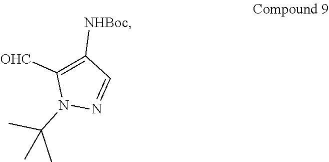 [SMILES: CC(C)(C)OC(=O)Nc1cnn(C(C)(C)C)c1C=O]
or a salt thereof, in the presence of A2, wherein A2 is an acid.

102. The process of claim 101, wherein A2 is a sulfonic acid.

103. The process of claim 101, wherein A2 is methanesulfonic acid.

104. The process of claim 101, wherein Compound 8 or a salt thereof is prepared by a process comprising: treating Compound 10 having the formula: 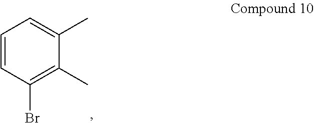
or a salt thereof, with magnesium in the presence of 2-methoxyacetonitrile.

105. The process of claim 104 wherein the treating of Compound 10 with magnesium is performed in presence of iodine.

106. The process of claim 101, wherein Compound 9 or a salt thereof is prepared by a process comprising: reacting Compound 11 having the formula: 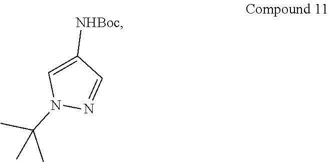 [SMILES: CC(C)(C)OC(=O)Nc1cnn(C(C)(C)C)c1]
or a salt thereof, with B3 followed by N,N-dimethylformamide, wherein B3 is a base.

107. The process of claim 106, wherein B3 is an organolithium base.

108. The process of claim 106, wherein B3 is n-butyllithium.

109. The process of claim 106, wherein Compound 11 or a salt thereof is prepared by a process comprising: reducing Compound 12 having the formula: 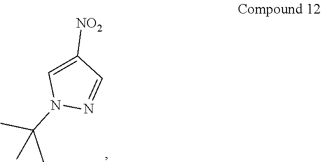
or a salt thereof, in the presence of di-tert-butyl dicarbonate and CA2, wherein CA2 is a catalyst.

110. The process of claim 109, wherein the reducing of Compound 12 is performed under a hydrogen atmosphere.

111. The process of claim 109, wherein CA2 is a hydrogenation catalyst.

112. The process of claim 109, wherein CA2 is 10% palladium on carbon.

113. The process of claim 109, wherein Compound 12 or a salt thereof is prepared by a process comprising: treating Compound 13 having the formula: 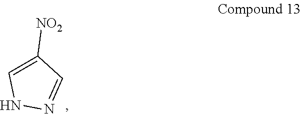
or a salt thereof, with tert-butyl acetate in the presence of A3, wherein A3 is an acid.

114. The process of claim 113, wherein A3 is an inorganic acid.

115. The process of claim 113, wherein A3 is sulfuric acid.

116. A compound selected from the following: 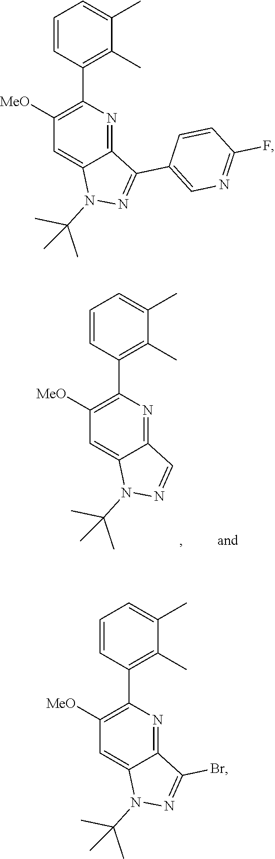 [SMILES: COc1cc2c(nc1-c1cccc(C)c1C)c(-c1ccc(F)nc1)nn2C(C)(C)C ; COc1cc2c(cnn2C(C)(C)C)nc1-c1cccc(C)c1C ; COc1cc2c(nc1-c1cccc(C)c1C)c(Br)nn2C(C)(C)C]
or a salt of any of the aforementioned.

117. A pharmaceutical composition comprising a solid form of claim 1 and a pharmaceutically acceptable carrier or excipient.

118. A method of inhibiting an FGFR3 enzyme, said method comprising contacting a solid form of claim 1 with said enzyme.

119. The method of claim 118, wherein the contacting comprises administering the solid form to a patient.

120. A method for treating a cancer in a patient, said method comprising: administering to the patient a therapeutically effective amount of a solid form of claim 1.

121. The method of claim 120, wherein the cancer is selected from adenocarcinoma, bladder cancer, breast cancer, cervical cancer, cholangiocarcinoma, endometrial cancer, gastric cancer, glioma, head and neck cancer, lung cancer, ovarian cancer, leukemia, and multiple myeloma.

122. A pharmaceutical composition comprising a salt of claim 17 and a pharmaceutically acceptable carrier or excipient.

123. A method of inhibiting an FGFR3 enzyme, said method comprising contacting a salt of claim 17 with said enzyme.

124. The method of claim 123, wherein the contacting comprises administering the salt to a patient.

125. A method for treating a cancer in a patient, said method comprising: administering to the patient a therapeutically effective amount of a salt of claim 17.

126. The method of claim 125, wherein the cancer is selected from adenocarcinoma, bladder cancer, breast cancer, cervical cancer, cholangiocarcinoma, endometrial cancer, gastric cancer, glioma, head and neck cancer, lung cancer, ovarian cancer, leukemia, and multiple myeloma.
